SEQUENCE LISTING (472 sequences)

SEQ ID NO: 1 - protein, 5 aa, synthetic construct
YYGIS

SEQ ID NO: 2 - protein, 5 aa, synthetic construct
DTYMH

SEQ ID NO: 3 - protein, 5 aa, synthetic construct
GYYWN

SEQ ID NO: 4 - protein, 5 aa, synthetic construct
SYDIN

SEQ ID NO: 5 - protein, 5 aa, synthetic construct
SYWMS

SEQ ID NO: 6 - protein, 5 aa, synthetic construct
GYYMH

SEQ ID NO: 7 - protein, 5 aa, synthetic construct
DYHWA

SEQ ID NO: 8 - protein, 5 aa, synthetic construct
GFYWS

SEQ ID NO: 9 - protein, 5 aa, synthetic construct
DYFWT

SEQ ID NO: 10 - protein, 5 aa, synthetic construct
DYYMS

SEQ ID NO: 11 - protein, 5 aa, synthetic construct
SSWMH

SEQ ID NO: 12 - protein, 5 aa, synthetic construct
SYDIN

SEQ ID NO: 13 - protein, 5 aa, synthetic construct
DTYMH

SEQ ID NO: 14 - protein, 5 aa, synthetic construct
DTYMH

SEQ ID NO: 15 - protein, 5 aa, synthetic construct
DTYMH

SEQ ID NO: 16 - protein, 5 aa, synthetic construct
DTYMH

SEQ ID NO: 17 - protein, 5 aa, synthetic construct
TYGMS

SEQ ID NO: 18 - protein, 7 aa, synthetic construct
SRSYYWG

SEQ ID NO: 19 - protein, 17 aa, synthetic construct
WISTYNGNTHYARMLQG

SEQ ID NO: 20 - protein, 17 aa, synthetic construct
RIDPASGHTKYDPKFQV

SEQ ID NO: 21 - protein, 16 aa, synthetic construct
EINHAGNTNYNPSLKS

SEQ ID NO: 22 - protein, 17 aa, synthetic construct
WLNPNSGYTGYAQKFQG

SEQ ID NO: 23 - protein, 17 aa, synthetic construct
NIKEDGSEKNYVDSVKG

SEQ ID NO: 24 - protein, 17 aa, synthetic construct
WIKPKSGGTNYVQKFQG

SEQ ID NO: 25 - protein, 16 aa, synthetic construct
DINHRGRTNYNPSLKS

SEQ ID NO: 26 - protein, 16 aa, synthetic construct
EINHRGNTNYNPSLKS

SEQ ID NO: 27 - protein, 16 aa, synthetic construct
EISHSGRTNYNPSLKS

SEQ ID NO: 28 - protein, 17 aa, synthetic construct
YIGGSGSTIYYADSVKG

SEQ ID NO: 29 - protein, 17 aa, synthetic construct
EIHPNSGGTNYAQKFQG

SEQ ID NO: 30 - protein, 17 aa, synthetic construct
WLNPNSGNTGYAQKFQG

SEQ ID NO: 31 - protein, 17 aa, synthetic construct
RIDPASGHTKYDPKFQV

SEQ ID NO: 32 - protein, 17 aa, synthetic construct
RIDPASGHTKYDPKFQV

SEQ ID NO: 33 - protein, 17 aa, synthetic construct
RIDPASGHTKYDPKFQV

SEQ ID NO: 34 - protein, 17 aa, synthetic construct
RIDPASGHTKYDPKFQV

SEQ ID NO: 35 - protein, 17 aa, synthetic construct
WMNTYSGVTTYADDFKG

SEQ ID NO: 36 - protein, 16 aa, synthetic construct
SIYYNGRTYYNPSLKS

SEQ ID NO: 37 - protein, 15 aa, synthetic construct
ENYYGSGAYRGGMDV

SEQ ID NO: 38 - protein, 7 aa, synthetic construct
SGGLPDV

SEQ ID NO: 39 - protein, 12 aa, synthetic construct
GYCRSTTCYFDY

SEQ ID NO: 40 - protein, 10 aa, synthetic construct
EVPETAAFEY

SEQ ID NO: 41 - protein, 13 aa, synthetic construct
EDYDSYYKYGMDV

SEQ ID NO: 42 - protein, 9 aa, synthetic construct
GGSQDASDF

SEQ ID NO: 43 - protein, 9 aa, synthetic construct
DFPNWVFDS

SEQ ID NO: 44 - protein, 11 aa, synthetic construct
PFYDFWSGSDY

SEQ ID NO: 45 - protein, 9 aa, synthetic construct
DYPNWVFDY

SEQ ID NO: 46 - protein, 13 aa, synthetic construct
EDADSYYYYGMDV

SEQ ID NO: 47 - protein, 12 aa, synthetic construct
GDYYGYVSWFAY

SEQ ID NO: 48 - protein, 10 aa, synthetic construct
EVPETAAFEY

SEQ ID NO: 49 - protein, 7 aa, synthetic construct
SGGLPDV

SEQ ID NO: 50 - protein, 7 aa, synthetic construct
SGGLPDV

SEQ ID NO: 51 - protein, 7 aa, synthetic construct
SGGLPDV

SEQ ID NO: 52 - protein, 7 aa, synthetic construct
SGGLPDV

SEQ ID NO: 53 - protein, 13 aa, synthetic construct
EGYVFDDYYATDY

SEQ ID NO: 54 - protein, 11 aa, synthetic construct
EDYGDYGAFDI

SEQ ID NO: 55 - protein, 11 aa, synthetic construct
RASQSVSSYLA

SEQ ID NO: 56 - protein, 10 aa, synthetic construct
RASSSVSYMY

SEQ ID NO: 57 - protein, 12 aa, synthetic construct
RASQSVRSSYLA

SEQ ID NO: 58 - protein, 14 aa, synthetic construct
TSSSSDIGAGLGVH

SEQ ID NO: 59 - protein, 17 aa, synthetic construct
KSSQSILYSSNNKNYLA

SEQ ID NO: 60 - protein, 11 aa, synthetic construct
RASQDISIWLA

SEQ ID NO: 61 - protein, 12 aa, synthetic construct
RASQSVSSSFLA

SEQ ID NO: 62 - protein, 16 aa, synthetic construct
KSSQSLVHSDGNTYLS

SEQ ID NO: 63 - protein, 12 aa, synthetic construct
RASQSVFNSYLA

SEQ ID NO: 64 - protein, 17 aa, synthetic construct
KSSQSVLYSSNNKNHLS

SEQ ID NO: 65 - protein, 11 aa, synthetic construct
QASQNINVLLN

SEQ ID NO: 66 - protein, 14 aa, synthetic construct
TSSSSDIGAGLGVH

SEQ ID NO: 67 - protein, 10 aa, synthetic construct
RASSSVSYMY

SEQ ID NO: 68 - protein, 10 aa, synthetic construct
RASSSVSYMY

SEQ ID NO: 69 - protein, 10 aa, synthetic construct
RASSSVSYMY

SEQ ID NO: 70 - protein, 10 aa, synthetic construct
RASSSVSYMY

SEQ ID NO: 71 - protein, 16 aa, synthetic construct
RSSQNIVHSDGNTYLE

SEQ ID NO: 72 - protein, 11 aa, synthetic construct
RASQGISSALA

SEQ ID NO: 73 - protein, 7 aa, synthetic construct
DASNRAT

SEQ ID NO: 74 - protein, 7 aa, synthetic construct
ATSNLAS

SEQ ID NO: 75 - protein, 7 aa, synthetic construct
GASSRAT

SEQ ID NO: 76 - protein, 7 aa, synthetic construct
GYYNRPS

SEQ ID NO: 77 - protein, 7 aa, synthetic construct
WASTRES

SEQ ID NO: 78 - protein, 7 aa, synthetic construct
AASSLQS

SEQ ID NO: 79 - protein, 7 aa, synthetic construct
GASRRAT

SEQ ID NO: 80 - protein, 7 aa, synthetic construct
KISNRFS

SEQ ID NO: 81 - protein, 7 aa, synthetic construct
GASRRAT

SEQ ID NO: 82 - protein, 7 aa, synthetic construct
WASTRES

SEQ ID NO: 83 - protein, 7 aa, synthetic construct
KASNLHT

SEQ ID NO: 84 - protein, 7 aa, synthetic construct
GYYNRPS

SEQ ID NO: 85 - protein, 7 aa, synthetic construct
ATSNLAS

SEQ ID NO: 86 - protein, 7 aa, synthetic construct
ATSNLAS

SEQ ID NO: 87 - protein, 7 aa, synthetic construct
ATSNLAS

SEQ ID NO: 88 - protein, 7 aa, synthetic construct
ATSNLAS

SEQ ID NO: 89 - protein, 7 aa, synthetic construct
KVSNRFS

SEQ ID NO: 90 - protein, 7 aa, synthetic construct
DASSLES

SEQ ID NO: 91 - protein, 9 aa, synthetic construct
QQRSNWPWT

SEQ ID NO: 92 - protein, 9 aa, synthetic construct
QQWEGNPRT

SEQ ID NO: 93 - protein, 8 aa, synthetic construct
QQYGSSPT

SEQ ID NO: 94 - protein, 10 aa, synthetic construct
QSWDGTLSAL

SEQ ID NO: 95 - protein, 9 aa, synthetic construct
QQYYSTPFT

SEQ ID NO: 96 - protein, 9 aa, synthetic construct
QQADSFPLT

SEQ ID NO: 97 - protein, 9 aa, synthetic construct
QQYGSSPLT

SEQ ID NO: 98 - protein, 9 aa, synthetic construct
MQATQFPLT

SEQ ID NO: 99 - protein, 9 aa, synthetic construct
QQYGNSPLT

SEQ ID NO: 100 - protein, 9 aa, synthetic construct
QQYYSTPRT

SEQ ID NO: 101 - protein, 9 aa, synthetic construct
QQGQSYPYT

SEQ ID NO: 102 - protein, 10 aa, synthetic construct
QSYDGTLSAL

SEQ ID NO: 103 - protein, 9 aa, synthetic construct
QQWSGNPRT

SEQ ID NO: 104 - protein, 9 aa, synthetic construct
QQWSGNPRT

SEQ ID NO: 105 - protein, 9 aa, synthetic construct
QQWSGNPRT

SEQ ID NO: 106 - protein, 9 aa, synthetic construct
QQWSGNPRT

SEQ ID NO: 107 - protein, 9 aa, synthetic construct
FQGSHVPLT

SEQ ID NO: 108 - protein, 9 aa, synthetic construct
QQFNSYPLT

SEQ ID NO: 109 - protein, 7 aa, synthetic construct
GYDFTYY

SEQ ID NO: 110 - protein, 7 aa, synthetic construct
GFDIQDT

SEQ ID NO: 111 - protein, 7 aa, synthetic construct
GGSFSGY

SEQ ID NO: 112 - protein, 7 aa, synthetic construct
GYTFTSY

SEQ ID NO: 113 - protein, 7 aa, synthetic construct
GFTFSSY

SEQ ID NO: 114 - protein, 7 aa, synthetic construct
GYTFTGY

SEQ ID NO: 115 - protein, 7 aa, synthetic construct
GVSFSDY

SEQ ID NO: 116 - protein, 7 aa, synthetic construct
GGSFTGF

SEQ ID NO: 117 - protein, 7 aa, synthetic construct
GGSFSDY

SEQ ID NO: 118 - protein, 7 aa, synthetic construct
GFTISDY

SEQ ID NO: 119 - protein, 7 aa, synthetic construct
GYTFTSS

SEQ ID NO: 120 - protein, 7 aa, synthetic construct
GYTFTSY

SEQ ID NO: 121 - protein, 7 aa, synthetic construct
GFDICDT

SEQ ID NO: 122 - protein, 7 aa, synthetic construct
GFDICDT

SEQ ID NO: 123 - protein, 7 aa, synthetic construct
GFDICDT

SEQ ID NO: 124 - protein, 7 aa, synthetic construct
GFDICDT

SEQ ID NO: 125 - protein, 7 aa, synthetic construct
GYTFTTY

SEQ ID NO: 126 - protein, 9 aa, synthetic construct
GGSISSRSY

SEQ ID NO: 127 - protein, 6 aa, synthetic construct
STYNGN

SEQ ID NO: 128 - protein, 6 aa, synthetic construct
DPASGH

SEQ ID NO: 129 - protein, 5 aa, synthetic construct
NHAGN

SEQ ID NO: 130 - protein, 6 aa, synthetic construct
NPNSGY

SEQ ID NO: 131 - protein, 6 aa, synthetic construct
KEDGSE

SEQ ID NO: 132 - protein, 6 aa, synthetic construct
KPKSGG

SEQ ID NO: 133 - protein, 5 aa, synthetic construct
NHRGR

SEQ ID NO: 134 - protein, 5 aa, synthetic construct
NHRGN

SEQ ID NO: 135 - protein, 5 aa, synthetic construct
SHSGR

SEQ ID NO: 136 - protein, 6 aa, synthetic construct
GGSGST

SEQ ID NO: 137 - protein, 6 aa, synthetic construct
HPNSGG

SEQ ID NO: 138 - protein, 6 aa, synthetic construct
NPNSGN

SEQ ID NO: 139 - protein, 6 aa, synthetic construct
DPASGH

SEQ ID NO: 140 - protein, 6 aa, synthetic construct
DPASGH

SEQ ID NO: 141 - protein, 6 aa, synthetic construct
DPASGH

SEQ ID NO: 142 - protein, 6 aa, synthetic construct
DPASGH

SEQ ID NO: 143 - protein, 6 aa, synthetic construct
NTYSGV

SEQ ID NO: 144 - protein, 5 aa, synthetic construct
YYNGR

SEQ ID NO: 145 - protein, 13 aa, synthetic construct
NYYGSGAYRGGMD

SEQ ID NO: 146 - protein, 5 aa, synthetic construct
GGLPD

SEQ ID NO: 147 - protein, 10 aa, synthetic construct
YCRSTTCYFD

SEQ ID NO: 148 - protein, 8 aa, synthetic construct
VPETAAFE

SEQ ID NO: 149 - protein, 11 aa, synthetic construct
DYDSYYKYGMD

SEQ ID NO: 150 - protein, 7 aa, synthetic construct
GSQDASD

SEQ ID NO: 151 - protein, 7 aa, synthetic construct
FPNWVFD

SEQ ID NO: 152 - protein, 9 aa, synthetic construct
FYDFWSGSD

SEQ ID NO: 153 - protein, 7 aa, synthetic construct
YPNWVFD

SEQ ID NO: 154 - protein, 11 aa, synthetic construct
DADSYYYYGMD

SEQ ID NO: 155 - protein, 10 aa, synthetic construct
DYYGYVSWFA

SEQ ID NO: 156 - protein, 8 aa, synthetic construct
VPETAAFE

SEQ ID NO: 157 - protein, 5 aa, synthetic construct
GGLPD

SEQ ID NO: 158 - protein, 5 aa, synthetic construct
GGLPD

SEQ ID NO: 159 - protein, 5 aa, synthetic construct
GGLPD

SEQ ID NO: 160 - protein, 5 aa, synthetic construct
GGLPD

SEQ ID NO: 161 - protein, 11 aa, synthetic construct
GYVFDDYYATD

SEQ ID NO: 162 - protein, 11 aa, synthetic construct
EDYGDYGAFDI

SEQ ID NO: 163 - protein, 7 aa, synthetic construct
SQSVSSY

SEQ ID NO: 164 - protein, 6 aa, synthetic construct
SSSVSY

SEQ ID NO: 165 - protein, 8 aa, synthetic construct
SQSVRSSY

SEQ ID NO: 166 - protein, 11 aa, synthetic construct
SSSSDIGAGLG

SEQ ID NO: 167 - protein, 13 aa, synthetic construct
SQSILYSSNNKNY

SEQ ID NO: 168 - protein, 7 aa, synthetic construct
SQDISIW

SEQ ID NO: 169 - protein, 8 aa, synthetic construct
SQSVSSSF

SEQ ID NO: 170 - protein, 12 aa, synthetic construct
SQSLVHSDGNTY

SEQ ID NO: 171 - protein, 8 aa, synthetic construct
SQSVFNSY

SEQ ID NO: 172 - protein, 13 aa, synthetic construct
SQSVLYSSNNKNH

SEQ ID NO: 173 - protein, 7 aa, synthetic construct
SQNINVL

SEQ ID NO: 174 - protein, 11 aa, synthetic construct
SSSSDIGAGLG

SEQ ID NO: 175 - protein, 6 aa, synthetic construct
SSSVSY

SEQ ID NO: 176 - protein, 6 aa, synthetic construct
SSSVSY

SEQ ID NO: 177 - protein, 6 aa, synthetic construct
SSSVSY

SEQ ID NO: 178 - protein, 6 aa, synthetic construct
SSSVSY

SEQ ID NO: 179 - protein, 12 aa, synthetic construct
SQNIVHSDGNTY

SEQ ID NO: 180 - protein, 11 aa, synthetic construct
RASQGISSALA

SEQ ID NO: 181-197 - skipped (17 entries)

SEQ ID NO: 198 - protein, 7 aa, synthetic construct
DASSLES

SEQ ID NO: 199 - protein, 6 aa, synthetic construct
RSNWPW

SEQ ID NO: 200 - protein, 6 aa, synthetic construct
WEGNPR

SEQ ID NO: 201 - protein, 5 aa, synthetic construct
YGSSP

SEQ ID NO: 202 - protein, 7 aa, synthetic construct
WDGTLSA

SEQ ID NO: 203 - protein, 6 aa, synthetic construct
YYSTPF

SEQ ID NO: 204 - protein, 6 aa, synthetic construct
ADSFPL

SEQ ID NO: 205 - protein, 6 aa, synthetic construct
YGSSPL

SEQ ID NO: 206 - protein, 6 aa, synthetic construct
ATQFPL

SEQ ID NO: 207 - protein, 6 aa, synthetic construct
YGNSPL

SEQ ID NO: 208 - protein, 6 aa, synthetic construct
YYSTPR

SEQ ID NO: 209 - protein, 6 aa, synthetic construct
GQSYPY

SEQ ID NO: 210 - protein, 7 aa, synthetic construct
YDGTLSA

SEQ ID NO: 211 - protein, 6 aa, synthetic construct
WSGNPR

SEQ ID NO: 212 - protein, 6 aa, synthetic construct
WSGNPR

SEQ ID NO: 213 - protein, 6 aa, synthetic construct
WSGNPR

SEQ ID NO: 214 - protein, 6 aa, synthetic construct
WSGNPR

SEQ ID NO: 215 - protein, 6 aa, synthetic construct
GSHVPL

SEQ ID NO: 216 - protein, 9 aa, synthetic construct
QQFNSYPLT

SEQ ID NO: 217 - protein, 8 aa, synthetic construct
GYDFTYYG

SEQ ID NO: 218 - protein, 8 aa, synthetic construct
GFDIQDTY

SEQ ID NO: 219 - protein, 8 aa, synthetic construct
GGSFSGYY

SEQ ID NO: 220 - protein, 8 aa, synthetic construct
GYTFTSYD

SEQ ID NO: 221 - protein, 8 aa, synthetic construct
GFTFSSYW

SEQ ID NO: 222 - protein, 8 aa, synthetic construct
GYTFTGYY

SEQ ID NO: 223 - protein, 8 aa, synthetic construct
GVSFSDYH

SEQ ID NO: 224 - protein, 8 aa, synthetic construct
GGSFTGFY

SEQ ID NO: 225 - protein, 8 aa, synthetic construct
GGSFSDYF

SEQ ID NO: 226 - protein, 8 aa, synthetic construct
GFTISDYY

SEQ ID NO: 227 - protein, 8 aa, synthetic construct
GYTFTSSW

SEQ ID NO: 228 - protein, 8 aa, synthetic construct
GYTFTSYD

SEQ ID NO: 229 - protein, 8 aa, synthetic construct
GFDICDTY

SEQ ID NO: 230 - protein, 8 aa, synthetic construct
GFDICDTY

SEQ ID NO: 231 - protein, 8 aa, synthetic construct
GFDICDTY

SEQ ID NO: 232 - protein, 8 aa, synthetic construct
GFDICDTY

SEQ ID NO: 233 - protein, 8 aa, synthetic construct
GYTFTTYG

SEQ ID NO: 234 - protein, 10 aa, synthetic construct
GGSISSRSYY

SEQ ID NO: 235 - protein, 8 aa, synthetic construct
ISTYNGNT

SEQ ID NO: 236 - protein, 8 aa, synthetic construct
IDPASGHT

SEQ ID NO: 237 - protein, 7 aa, synthetic construct
INHAGNT

SEQ ID NO: 238 - protein, 8 aa, synthetic construct
LNPNSGYT

SEQ ID NO: 239 - protein, 8 aa, synthetic construct
IKEDGSEK

SEQ ID NO: 240 - protein, 8 aa, synthetic construct
IKPKSGGT

SEQ ID NO: 241 - protein, 7 aa, synthetic construct
INHRGRT

SEQ ID NO: 242 - protein, 7 aa, synthetic construct
INHRGNT

SEQ ID NO: 243 - protein, 7 aa, synthetic construct
ISHSGRT

SEQ ID NO: 244 - protein, 8 aa, synthetic construct
IGGSGSTI

SEQ ID NO: 245 - protein, 8 aa, synthetic construct
IHPNSGGT

SEQ ID NO: 246 - protein, 8 aa, synthetic construct
LNPNSGNT

SEQ ID NO: 247 - protein, 8 aa, synthetic construct
IDPASGHT

SEQ ID NO: 248 - protein, 8 aa, synthetic construct
IDPASGHT

SEQ ID NO: 249 - protein, 8 aa, synthetic construct
IDPASGHT

SEQ ID NO: 250 - protein, 8 aa, synthetic construct
IDPASGHT

SEQ ID NO: 251 - protein, 8 aa, synthetic construct
MNTYSGVT

SEQ ID NO: 252 - protein, 7 aa, synthetic construct
IYYNGRT

SEQ ID NO: 253 - protein, 17 aa, synthetic construct
ARENYYGSGAYRGGMDV

SEQ ID NO: 254 - protein, 9 aa, synthetic construct
ARSGGLPDV

SEQ ID NO: 255 - protein, 14 aa, synthetic construct
ARGYCRSTTCYFDY

SEQ ID NO: 256 - protein, 12 aa, synthetic construct
AREVPETAAFEY

SEQ ID NO: 257 - protein, 15 aa, synthetic construct
AREDYDSYYKYGMDV

SEQ ID NO: 258 - protein, 11 aa, synthetic construct
ATGGSQDASDF

SEQ ID NO: 259 - protein, 11 aa, synthetic construct
ARDFPNWVFDS

SEQ ID NO: 260 - protein, 13 aa, synthetic construct
ASPFYDFWSGSDY

SEQ ID NO: 261 - protein, 11 aa, synthetic construct
ARDYPNWVFDY

SEQ ID NO: 262 - protein, 15 aa, synthetic construct
AREDADSYYYYGMDV

SEQ ID NO: 263 - protein, 14 aa, synthetic construct
ARGDYYGYVSWFAY

SEQ ID NO: 264 - protein, 12 aa, synthetic construct
AREVPETAAFEY

SEQ ID NO: 265 - protein, 9 aa, synthetic construct
SRSGGLPDV

SEQ ID NO: 266 - protein, 9 aa, synthetic construct
ARSGGLPDV

SEQ ID NO: 267 - protein, 9 aa, synthetic construct
ARSGGLPDV

SEQ ID NO: 268 - protein, 9 aa, synthetic construct
ARSGGLPDV

SEQ ID NO: 269 - protein, 15 aa, synthetic construct
AREGYVFDDYYATDY

SEQ ID NO: 270 - protein, 13 aa, synthetic construct
AREDYGDYGAFDI

SEQ ID NO: 271 - protein, 6 aa, synthetic construct
QSVSSY

SEQ ID NO: 272 - protein, 5 aa, synthetic construct
SSVSY

SEQ ID NO: 273 - protein, 7 aa, synthetic construct
QSVRSSY

SEQ ID NO: 274 - protein, 9 aa, synthetic construct
SSDIGAGLG

SEQ ID NO: 275 - protein, 12 aa, synthetic construct
QSILYSSNNKNY

SEQ ID NO: 276 - protein, 6 aa, synthetic construct
QDISIW

SEQ ID NO: 277 - protein, 7 aa, synthetic construct
QSVSSSF

SEQ ID NO: 278 - protein, 11 aa, synthetic construct
QSLVHSDGNTY

SEQ ID NO: 279 - protein, 7 aa, synthetic construct
QSVFNSY

SEQ ID NO: 280 - protein, 12 aa, synthetic construct
QSVLYSSNNKNH

SEQ ID NO: 281 - protein, 6 aa, synthetic construct
QNINVL

SEQ ID NO: 282 - protein, 9 aa, synthetic construct
SSDIGAGLG

SEQ ID NO: 283 - protein, 5 aa, synthetic construct
SSVSY

SEQ ID NO: 284 - protein, 5 aa, synthetic construct
SSVSY

SEQ ID NO: 285 - protein, 5 aa, synthetic construct
SSVSY

SEQ ID NO: 286 - protein, 5 aa, synthetic construct
SSVSY

SEQ ID NO: 287 - protein, 11 aa, synthetic construct
QNIVHSDGNTY

SEQ ID NO: 288 - protein, 6 aa, synthetic construct
QGISSA

SEQ ID NO: 289-306 - skipped (18 entries)

SEQ ID NO: 307 - protein, 9 aa, synthetic construct
QQRSNWPWT

SEQ ID NO: 308 - protein, 9 aa, synthetic construct
QQWEGNPRT

SEQ ID NO: 309 - protein, 8 aa, synthetic construct
QQYGSSPT

SEQ ID NO: 310 - protein, 10 aa, synthetic construct
QSWDGTLSAL

SEQ ID NO: 311 - protein, 9 aa, synthetic construct
QQYYSTPFT

SEQ ID NO: 312 - protein, 9 aa, synthetic construct
QQADSFPLT

SEQ ID NO: 313 - protein, 9 aa, synthetic construct
QQYGSSPLT

SEQ ID NO: 314 - protein, 9 aa, synthetic construct
MQATQFPLT

SEQ ID NO: 315 - protein, 9 aa, synthetic construct
QQYGNSPLT

SEQ ID NO: 316 - protein, 9 aa, synthetic construct
QQYYSTPRT

SEQ ID NO: 317 - protein, 9 aa, synthetic construct
QQGQSYPYT

SEQ ID NO: 318 - protein, 10 aa, synthetic construct
QSYDGTLSAL

SEQ ID NO: 319 - protein, 9 aa, synthetic construct
QQWSGNPRT

SEQ ID NO: 320 - protein, 9 aa, synthetic construct
QQWSGNPRT

SEQ ID NO: 321 - protein, 9 aa, synthetic construct
QQWSGNPRT

SEQ ID NO: 322 - protein, 9 aa, synthetic construct
QQWSGNPRT

SEQ ID NO: 323 - protein, 9 aa, synthetic construct
FQGSHVPLT

SEQ ID NO: 324 - protein, 9 aa, synthetic construct
QQFNSYPLT

SEQ ID NO: 325 - protein, 124 aa, synthetic construct
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGAYRGGMDVWGQGTTVTVSS

SEQ ID NO: 326 - protein, 116 aa, synthetic construct
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSS

SEQ ID NO: 327 - protein, 120 aa, synthetic construct
QVQLQQWGAGLLKPSETLSLTCAVHGGSFSGYYWNWIRQPPGKGLEWIGEINHAGNTNYNPSLKSRVTISLDTSKNQFSLTLTSVTAADTAVYYCARGYCRSTTCYFDYWGQGTLVTVSS

SEQ ID NO: 328 - protein, 119 aa, synthetic construct
QVQLVQSGAEVKKPGASVKVSCKASGYTFTSYDINWVRQAPGQGLEWMGWLNPNSGYTGYAQKFQGRVTMTADRSTSTAYMELSSLRSEDTAVYYCAREVPETAAFEYWGQGTLVTVSS

SEQ ID NO: 329 - protein, 122 aa, synthetic construct
EVQLVESGGGLVQPGGSLRLSCAVSGFTFSSYWMSWVRQAPGKGLEWVANIKEDGSEKNYVDSVKGRFTLSSDNAKNSLYLQMNSLRAEDTAVYYCAREDYDSYYKYGMDVWGQGTAVIVSS

SEQ ID NO: 330 - protein, 118 aa, synthetic construct
QVQLVESGAEVKKPGASVKVSCKASGYTFTGYYMHWVRQAPGQGLEWMGWIKPKSGGTNYVQKFQGRVTMTRDTSINTAYMELNNLKSDDTAVYYCATGGSQDASDFWGQGTMVTVSS

SEQ ID NO: 331 - protein, 117 aa, synthetic construct
QVQLHQWGAGLLKPSETLSLTCAVSGVSFSDYHWAWIRQSPGKGLEWIGDINHRGRTNYNPSLKSRVTISLDTSGNPFSLKLTSVTAADTAVYYCARDFPNWVFDSWGQGILVTVSS

SEQ ID NO: 332 - protein, 119 aa, synthetic construct
QVQLQQWGAGLLKPSETLSLTCAVYGGSFTGFYWSWIRQPPGKGLEWIGEINHRGNTNYNPSLKSRVTMSVDTSKNQFSLNMISVTAADTAMYFCASPFYDFWSGSDYWGQGTLVTVSS

SEQ ID NO: 333 - protein, 117 aa, synthetic construct
QVQLQQWGAGLLKPSETLSLTCAVSGGSFSDYFWTWIRQPPGKGLEWIGEISHSGRTNYNPSLKSRVTISVDTSMSQFSLKMTSVTAADSAVYYCARDYPNWVFDYWGQGTLVTVSS

SEQ ID NO: 334 - protein, 122 aa, synthetic construct
RLQLVESGGGLVKPGGSLKLSCAASGFTISDYYMSWIRQAPGKGLEWVSYIGGSGSTIYYADSVKGRFTISRDNAKNSLYLQMNSLRAEDTAVYYCAREDADSYYYYGMDVWGQGTTVTVSS

SEQ ID NO: 335 - protein, 121 aa, synthetic construct
QVQLVQSGAEVKKPGASVKVSCKASGYTFTSSWMHWARQAPGQGLEWMGEIHPNSGGTNYAQKFQGRVTMTVDTSISTAYMELSRLRSDDTAVYYCARGDYYGYVSWFAYWGQGTLVTVSS

SEQ ID NO: 336 - protein, 119 aa, synthetic construct
QVQLVQSGAEVKKPGASVKVSCKASGYTFTSYDINWVRQAPGQGLEWMGWLNPNSGNTGYAQKFQGRVTMTRNTSISTAYMELSSLRSEDTAVYYCAREVPETAAFEYWGQGTLVTVSS

SEQ ID NO: 337 - protein, 116 aa, synthetic construct
QVQLVQSGAEVKKPGASVKVSCKASGFDICDTYMHWVKQRPGQGLEWIGRIDPASGHTKYDPKFQVRATITRDTSTSTAYLELSSLRSEDTAVYYCSRSGGLPDVWGQGTTVTVSS

SEQ ID NO: 338 - protein, 116 aa, synthetic construct
QVQLVQSGAEVVKPGASVKLSCKASGFDICDTYMHWVRQRPGQGLEWMGRIDPASGHTKYDPKFQVRVTMTTDTSTSTVYLELSSLRSEDTAVYYCARSGGLPDVWGQGTTVTVSS

SEQ ID NO: 339 - protein, 116 aa, synthetic construct
QVQLVQSGAEVKKPGASVKLSCKASGFDICDTYMHWVRQRPGQGLEWIGRIDPASGHTKYDPKFQVRATMTTDTSTSTVYLELSSLRSEDTAVYYCARSGGLPDVWGQGTTVTVSS

SEQ ID NO: 340 - protein, 116 aa, synthetic construct
QVQLVQSGAEVKKPGASVKVSCKASGFDICDTYMHWVKQRPGQGLEWIGRIDPASGHTKYDPKFQVRATITTDTSTSTAYLELSSLRSEDTAVYYCARSGGLPDVWGQGTTVTVSS

SEQ ID NO: 341 - protein, 122 aa, synthetic construct
QIQLVQSGPELKKPGETVKISCKASGYTFTTYGMSWVKQAPGKGLKWMGWMNTYSGVTTYADDFKGRFAFSLETSASTAYMQIDNLKNEDTATYFCAREGYVFDDYYATDYWGQGTSVTVSS

SEQ ID NO: 342 - protein, 121 aa, synthetic construct
QLQLQESGPGLVKPSETLSLTCTVSGGSISSRSYYWGWIRQPPGKGLEWIGSIYYNGRTYYNPSLKSRVTISVDTSKNQFSLKLSSVTAADTAVYYCAREDYGDYGAFDIWGQGTMVTVSS

SEQ ID NO: 343 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIK

SEQ ID NO: 344 - protein, 106 aa, synthetic construct
EIVLTQSPGTLSLSPGERATLSCRASSSVSYMYWYQQKPGQAPRPLIYATSNLASGIPDRFSGSGSGTDFTLTISRLEPEDFAVYYCQQWEGNPRTFGGGTKLEIK

SEQ ID NO: 345 - protein, 107 aa, synthetic construct
EIVLIQSPGILSLSPGERATLSCRASQSVRSSYLAWYQQKPGQAPRLLIYGASSRATGIPDRFSGSGSGTDFTLTISRLEPEDFAVYYCQQYGSSPTFGQGTRLEIK

SEQ ID NO: 346 - protein, 106 aa, synthetic construct
QSVLTQPPSVSGAPGQRVTISCTSSSSDIGAGLGVHWYQQLPGTAPKLLIEGYYNRPSGVPDRFSGTSASLTITGLLPEDEGDYYCQSWDGTLSALFGGGTKLTVL

SEQ ID NO: 347 - protein, 113 aa, synthetic construct
DIVMTQSPDSLAVSLGERATINCKSSQSILYSSNNKNYLAWYQQKPGQPPKLLIYWASTRESGVPDRFSGSGSGTDFTLTISSLQAEDVSVYYCQQYYSTPFTFGPGTKVDIK

SEQ ID NO: 348 - protein, 108 aa, synthetic construct
DIQLTQSPSSVSASVGGRVTITCRASQDISIWLAWYQQKPGKAPNLLIYAASSLQSGVPSRFSGSGSGTDFTLTISSLQPEDFATYYCQQADSFPLTFGGGTKLEIKR

SEQ ID NO: 349 - protein, 108 aa, synthetic construct
EIVLSQSPGTLSLSPGERATLSCRASQSVSSSFLAWYQQYPGQAPRLLIYGASRRATGIPDRFRGSGSGTDFTLTISRLEPEDFAVYYCQQYGSSPLTFGGGTKVENK

SEQ ID NO: 350 - protein, 112 aa, synthetic construct
DIMLTQTPLTSPVTLGQPASISCKSSQSLVHSDGNTYLSWLQQRPGQPPRLLFYKISNRFSGVPDRFSGSGAGTDFTLKISRVEAEDVGVYYCMQATQFPLTFGGGTKVEIK

SEQ ID NO: 351 - protein, 108 aa, synthetic construct
EIELTQSPGTLSLSPGEGATLSCRASQSVFNSYLAWYQQKPGQAPRLLIYGASRRATGIPDRFSGSGSGTDFTLTISRLEPGDFAVYFCQQYGNSPLTFGGGTKVEIK

SEQ ID NO: 352 - protein, 113 aa, synthetic construct
DIVMTQSPDSLAVSLGERATINCKSSQSVLYSSNNKNHLSWYQQKPGQPPKLLIYWASTRESGVPDRFSGSGSGTDFTLTISSLQAEDVAVYYCQQYYSTPRTFGQGTKVEIK

SEQ ID NO: 353 - protein, 107 aa, synthetic construct
DIQMTQSPSSLSASVGDRVTITCQASQNINVLLNWYQQKPGKAPKLLIYKASNLHTGVPSRFSGSGSGTDFTFTISSLQPEDIATYYCQQGQSYPYTFGQGTKLEIK

SEQ ID NO: 354 - protein, 110 aa, synthetic construct
QSVLTQPPSVSGAPGQRVTISCTSSSSDIGAGLGVHWYQQLPGTAPKLLIYGYYNRPSGVPDRFSGSKSGTSASLTITGLLPEDEGDYYCQSYDGTLSALFGGGTKLTVL

SEQ ID NO: 355 - protein, 106 aa, synthetic construct
EIVLTQSPGTLSASPGERVTLSCRASSSVSYMYWYQQKPGQAPRPWIYATSNLASGVPDRFSGSGSGTDFTLTISRLEPEDFAVYYCQQWSGNPRTFGGGTKLEIK

SEQ ID NO: 356 - protein, 106 aa, synthetic construct
EIVLTQSPGTLSLSPGERVTMSCRASSSVSYMYWYQQKPGQAPRPWIYATSNLASGVPDRFSGSGSGTDFTLTISRVEPEDFAVYYCQQWSGNPRTFGGGTKVEIK

SEQ ID NO: 357 - protein, 106 aa, synthetic construct
EIVLTQSPGTLSLSPGERATMSCRASSSVSYMYWYQQKPGQAPRPWIYATSNLASGIPDRFSGSGSGTDFTLTISRVEPEDFAVYYCQQWSGNPRTFGGGTKLEIK

SEQ ID NO: 358 - protein, 106 aa, synthetic construct
EIVLTQSPGTLSASPGERATMSCRASSSVSYMYWYQQKPGQAPRPWIYATSNLASGVPDRFSGSGSGTDYTLTISRVEPEDFAVYYCQQWSGNPRTFGGGTKLEIK

SEQ ID NO: 359 - protein, 110 aa, synthetic construct
DVLMTQTPLSLPVSLGDQASISCRSSQNIVHSDGNTYLEWYLQKPGQSPKWYKVSNRFSGVPDRFSGSGSGTDFTLKISRVEAEDLGIYYCFQGSHVPLTFGAGTKLELK

SEQ ID NO: 360 - protein, 107 aa, synthetic construct [sequence omitted]

SEQ ID NO: 361 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 362 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 363 - protein, 324 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSP

SEQ ID NO: 364 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 365 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 366 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 367 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 368 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 369 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 370 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 371 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 372 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 373 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 374 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 375 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 376 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 377 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 378 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 379 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 380 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 381 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 382 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 383 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 384 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 385 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 386 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 387 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 388 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 389 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 390 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 391 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 392 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 393 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 394 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 395 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 396 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSYTQKSLSLSLGK

SEQ ID NO: 397 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 398 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 399 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVDHHDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLGK

SEQ ID NO: 400 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVDHHDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLGK

SEQ ID NO: 401 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVDHHDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLGK

SEQ ID NO: 402 - protein, 324 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLYITREPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHSHYTQKSLSLSP

SEQ ID NO: 403 - protein, 324 aa, synthetic construct; identical to SEQ ID NO: 363

SEQ ID NO: 404 - protein, 324 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVDHHDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHSHYTQKSLSLSP

SEQ ID NO: 405 - protein, 327 aa, synthetic construct; identical to SEQ ID NO: 364

SEQ ID NO: 406 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 407 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 408 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 409 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 410 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 411 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 412 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 413 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 414 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 415 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 416 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 417 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 418 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 419 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 420 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 421 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 422 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 423 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 424 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 425 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 426 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 427 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 428 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYASTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 429 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVAVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 430 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 431 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 432 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 433 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 434 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 435 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYASTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 436 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVAVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 437 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 438 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 439 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 440 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLWVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 441 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 442 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRDPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 443 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRDPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 444 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 445 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 446 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 447 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYISRDPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 448 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 449 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 450 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 451 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 452 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 453 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 454 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALGAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 455 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 456 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVDNAKTKPREEQYASTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 457 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVAVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 458 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 459 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 460 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 461 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVDNAKTKPREEQYNSTYRVVSVLRVLHVDWLNGKEYKCKVSNKALGAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 462 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPG

SEQ ID NO: 463 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHRDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 464 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHRDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 465 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVLHRDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 466 - protein, 324 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHRDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHNHYTQKSLSLSP

SEQ ID NO: 467 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPG

SEQ ID NO: 468 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPG

SEQ ID NO: 469 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPG

SEQ ID NO: 470 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPG

SEQ ID NO: 471 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPG

SEQ ID NO: 472 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPG

(19) United States
(12) Patent Application Publication
(10) Pub. No.: US 2026/0234279 A1
(43) Pub. Date: Aug. 13, 2026

(54) TL1A ANTIBODY COMPOSITIONS AND METHODS OF USE

(71) Applicant: Paragon Therapeutics, Inc., Waltham, MA (US)

(72) Inventors: Eric Franklin Zhu, Waltham, MA (US); Byong Ha Kang, Waltham, MA (US)

(73) Assignee: Paragon Therapeutics, Inc., Waltham, MA (US)

(21) Appl. No.: 19/425,073

(22) Filed: Dec. 18, 2025

Related U.S. Application Data
(63) Continuation of application No. PCT/US2024/034949, filed on Jun. 21, 2024.
(60) Provisional application No. 63/509,463, filed on Jun. 21, 2023.

(51) Int. Cl. C07K 16/28; A61K 39/00

(52) U.S. Cl. CPC C07K 16/2875; A61K 2039/54; C07K 2317/52; C07K 2317/565; C07K 2317/94

(57) ABSTRACT

Provided herein are variant TL1A antibodies and methods of use.

### CROSS-REFERENCE TO RELATED APPLICATIONS

This application is a continuation application of PCT International Application No. PCT/US2024/034949, filed Jun. 21, 2024, which claims the benefit of and priority to U.S. Provisional Application No. 63/509,463, filed Jun. 21, 2023, the entire contents of each of which are incorporated herein by reference in their entireties.

### SEQUENCE LISTING

This application contains a Sequence Listing which has been submitted electronically in XML format and is hereby incorporated by reference in its entirety. The XML copy, created on Jun. 12, 2024, is titled PRG-006WO_SL.xml and is 426,107 bytes in size.

### BACKGROUND

Tumor necrosis factor (TNF)-like cytokine 1A (TL1A) is part of the TNF superfamily and is a transmembrane protein expressed by myeloid mononuclear cells and endothelial cells. TL1A interacts with its receptor, death receptor 3 (DR3) to trigger signaling. TL1A is elevated in individuals with inflammatory diseases including Crohn's disease and ulcerative colitis, and DR3 expression is upregulated in inflamed tissue. As such, TL1A along with other members of the TNF superfamily have been investigated as therapeutic targets to treat inflammatory diseases including inflammatory bowel diseases. Current biologics targeting TNF are associated with serious side effects highlighting the need for improved therapies targeting TL1A.

### SUMMARY OF THE DISCLOSURE

Described herein, in certain embodiments, are TL1A binding proteins comprising: a) a heavy chain variable region (VH) comprising (i) a CDR1 having an amino acid sequence according to any one of SEQ ID NOs: 1-18, (ii) a CDR2 having an amino acid sequence according to any one of SEQ ID NOs: 19-36, and (iii) a CDR3 having an amino acid sequence according to any one of SEQ ID NOs: 37-54; b) a light chain variable region (VL) comprising (i) a CDR1 having an amino acid sequence according to any one of SEQ ID NOs: 55-72, (ii) a CDR2 having an amino acid sequence according to any one of SEQ ID NOs: 73-90, and (iii) a CDR3 having an amino acid sequence according to any one of SEQ ID NOs: 91-108; and c) an Fc domain comprising amino acid modifications M252Y, S254T, and T256E (YTE) and/or M428L and N434S (LS). In some embodiments, the VH comprises (i) a CDR1 having an amino acid sequence according to SEQ ID NO: 1, (ii) a CDR2 having an amino acid sequence according to SEQ ID NO: 19, and (iii) a CDR3 having an amino acid sequence according to SEQ ID NO: 37; and the VL comprises (i) a CDR1 having an amino acid sequence according to SEQ ID NO: 55, (ii) a CDR2 having an amino acid sequence according to SEQ ID NO: 73, and (iii) a CDR3 having an amino acid sequence according to SEQ ID NO: 91. In some embodiments, the VH comprises (i) a CDR1 having an amino acid sequence according to SEQ ID NO: 2, (ii) a CDR2 having an amino acid sequence according to SEQ ID NO: 20, and (iii) a CDR3 having an amino acid sequence according to SEQ ID NO: 38; and the VL comprises (i) a CDR1 having an amino acid sequence according to SEQ ID NO: 56, (ii) a CDR2 having an amino acid sequence according to SEQ ID NO: 74, and (iii) a CDR3 having an amino acid sequence according to SEQ ID NO: 92. In some embodiments, the VH comprises (i) a CDR1 having an amino acid sequence according to SEQ ID NO: 3, (ii) a CDR2 having an amino acid sequence according to SEQ ID NO: 21, and (iii) a CDR3 having an amino acid sequence according to SEQ ID NO: 39; and the VL comprises (i) a CDR1 having an amino acid sequence according to SEQ ID NO: 57, (ii) a CDR2 having an amino acid sequence according to SEQ ID NO: 75, and (iii) a CDR3 having an amino acid sequence according to SEQ ID NO: 93. In some embodiments, the VH comprises (i) a CDR1 having an amino acid sequence according to SEQ ID NO: 4, (ii) a CDR2 having an amino acid sequence according to SEQ ID NO: 22, and (iii) a CDR3 having an amino acid sequence according to SEQ ID NO: 40; and the VL comprises (i) a CDR1 having an amino acid sequence according to SEQ ID NO: 58, (ii) a CDR2 having an amino acid sequence according to SEQ ID NO: 76, and (iii) a CDR3 having an amino acid sequence according to SEQ ID NO: 94. In some embodiments, the VH comprises (i) a CDR1 having an amino acid sequence according to SEQ ID NO: 5, (ii) a CDR2 having an amino acid sequence according to SEQ ID NO: 23, and (iii) a CDR3 having an amino acid sequence according to SEQ ID NO: 41; and the VL comprises (i) a CDR1 having an amino acid sequence according to SEQ ID NO: 59, (ii) a CDR2 having an amino acid sequence according to SEQ ID NO: 77, and (iii) a CDR3 having an amino acid sequence according to SEQ ID NO: 95. In some embodiments, the VH comprises (i) a CDR1 having an amino acid sequence according to SEQ ID NO: 6, (ii) a CDR2 having an amino acid sequence according to SEQ ID NO: 24, and (iii) a CDR3 having an amino acid sequence according to SEQ ID NO: 42; and the VL comprises (i) a CDR1 having an amino acid sequence according to SEQ ID NO: 60, (ii) a CDR2 having an amino acid sequence according to SEQ ID NO: 78, and (iii) a CDR3 having an amino acid sequence according to SEQ ID NO: 96. In some embodiments, the VH comprises (i) a CDR1 having an amino acid sequence according to SEQ ID NO: 7, (ii) a CDR2 having an amino acid sequence according to SEQ ID NO: 25, and (iii) a CDR3 having an amino acid sequence according to SEQ ID NO: 43; and the VL comprises (i) a CDR1 having an amino acid sequence according to SEQ ID NO: 61, (ii) a CDR2 having an amino acid sequence according to SEQ ID NO: 79, and (iii) a CDR3 having an amino acid sequence according to SEQ ID NO: 97. In some embodiments, the VH comprises (i) a CDR1 having an amino acid sequence according to SEQ ID NO: 8, (ii) a CDR2 having an amino acid sequence according to SEQ ID NO: 26, and (iii) a CDR3 having an amino acid sequence according to SEQ ID NO: 44; and the VL comprises (i) a CDR1 having an amino acid sequence according to SEQ ID NO: 62, (ii) a CDR2 having an amino acid sequence according to SEQ ID NO: 80, and (iii) a CDR3 having an amino acid sequence according to SEQ ID NO: 98. In some embodiments, the VH comprises (i) a CDR1 having an amino acid sequence according to SEQ ID NO: 9, (ii) a CDR2 having an amino acid sequence according to SEQ ID NO: 27, and (iii) a CDR3 having an amino acid sequence according to SEQ ID NO: 45; and the VL comprises (i) a CDR1 having an amino acid sequence according to SEQ ID NO: 63, (ii) a CDR2 having an amino acid sequence according to SEQ ID NO: 81, and (iii) a CDR3 having an amino acid sequence according to SEQ ID NO: 99. In some embodiments, the VH comprises (i) a CDR1 having an amino acid sequence according to SEQ ID NO: 10, (ii) a CDR2 having an amino acid sequence according to SEQ ID NO: 28, and (iii) a CDR3 having an amino acid sequence according to SEQ ID NO: 46; and the VL comprises (i) a CDR1 having an amino acid sequence according to SEQ ID NO: 64, (ii) a CDR2 having an amino acid sequence according to SEQ ID NO: 82, and (iii) a CDR3 having an amino acid sequence according to SEQ ID NO: 100. In some embodiments, the VH comprises (i) a CDR1 having an amino acid sequence according to SEQ ID NO: 11, (ii) a CDR2 having an amino acid sequence according to SEQ ID NO: 29, and (iii) a CDR3 having an amino acid sequence according to SEQ ID NO: 47; and the VL comprises (i) a CDR1 having an amino acid sequence according to SEQ ID NO: 65, (ii) a CDR2 having an amino acid sequence according to SEQ ID NO: 83, and (iii) a CDR3 having an amino acid sequence according to SEQ ID NO: 101. In some embodiments, the VH comprises (i) a CDR1 having an amino acid sequence according to SEQ ID NO: 12, (ii) a CDR2 having an amino acid sequence according to SEQ ID NO: 30, and (iii) a CDR3 having an amino acid sequence according to SEQ ID NO: 48; and the VL comprises (i) a CDR1 having an amino acid sequence according to SEQ ID NO: 66, (ii) a CDR2 having an amino acid sequence according to SEQ ID NO: 84, and (iii) a CDR3 having an amino acid sequence according to SEQ ID NO: 102. In some embodiments, the VH comprises (i) a CDR1 having an amino acid sequence according to SEQ ID NO: 13, (ii) a CDR2 having an amino acid sequence according to SEQ ID NO: 31, and (iii) a CDR3 having an amino acid sequence according to SEQ ID NO: 49; and the VL comprises (i) a CDR1 having an amino acid sequence according to SEQ ID NO: 67, (ii) a CDR2 having an amino acid sequence according to SEQ ID NO: 85, and (iii) a CDR3 having an amino acid sequence according to SEQ ID NO: 103. In some embodiments, the VH comprises (i) a CDR1 having an amino acid sequence according to SEQ ID NO: 14, (ii) a CDR2 having an amino acid sequence according to SEQ ID NO: 32, and (iii) a CDR3 having an amino acid sequence according to SEQ ID NO: 50; and the VL comprises (i) a CDR1 having an amino acid sequence according to SEQ ID NO: 68, (ii) a CDR2 having an amino acid sequence according to SEQ ID NO: 86, and (iii) a CDR3 having an amino acid sequence according to SEQ ID NO: 104. In some embodiments, the VH comprises (i) a CDR1 having an amino acid sequence according to SEQ ID NO: 15, (ii) a CDR2 having an amino acid sequence according to SEQ ID NO: 33, and (iii) a CDR3 having an amino acid sequence according to SEQ ID NO: 51; and the VL comprises (i) a CDR1 having an amino acid sequence according to SEQ ID NO: 69, (ii) a CDR2 having an amino acid sequence according to SEQ ID NO: 87, and (iii) a CDR3 having an amino acid sequence according to SEQ ID NO: 105. In some embodiments, the VH comprises (i) a CDR1 having an amino acid sequence according to SEQ ID NO: 16, (ii) a CDR2 having an amino acid sequence according to SEQ ID NO: 34, and (iii) a CDR3 having an amino acid sequence according to SEQ ID NO: 52; and the VL comprises (i) a CDR1 having an amino acid sequence according to SEQ ID NO: 70, (ii) a CDR2 having an amino acid sequence according to SEQ ID NO: 88, and (iii) a CDR3 having an amino acid sequence according to SEQ ID NO: 106. In some embodiments, the VH comprises (i) a CDR1 having an amino acid sequence according to SEQ ID NO: 17, (ii) a CDR2 having an amino acid sequence according to SEQ ID NO: 35, and (iii) a CDR3 having an amino acid sequence according to SEQ ID NO: 53; and the VL comprises (i) a CDR1 having an amino acid sequence according to SEQ ID NO: 71, (ii) a CDR2 having an amino acid sequence according to SEQ ID NO: 89, and (iii) a CDR3 having an amino acid sequence according to SEQ ID NO: 107. In some embodiments, the VH comprises (i) a CDR1 having an amino acid sequence according to SEQ ID NO: 18, (ii) a CDR2 having an amino acid sequence according to SEQ ID NO: 36, and (iii) a CDR3 having an amino acid sequence according to SEQ ID NO: 54; and the VL comprises (i) a CDR1 having an amino acid sequence according to SEQ ID NO: 72, (ii) a CDR2 having an amino acid sequence according to SEQ ID NO: 90, and (iii) a CDR3 having an amino acid sequence according to SEQ ID NO: 108. In some embodiments, the VH comprises a sequence having at least 80% sequence identity to the amino acid sequence of any one of SEQ ID NOs: 325-342 and the VL comprises a sequence having at least 80% sequence identity to the amino acid sequence of any one of SEQ ID NOs: 343-360. In some embodiments, the VH comprises a sequence having at least 80% sequence identity to the amino acid sequence of SEQ ID NO: 325 and the VL comprises a sequence having at least 80% sequence identity to the amino acid sequence of SEQ ID NO: 343. In some embodiments, the VH comprises a sequence having at least 80% sequence identity to the amino acid sequence of SEQ ID NO: 326 and the VL comprises a sequence having at least 80% sequence identity to the amino acid sequence of SEQ ID NO: 344. In some embodiments, the VH comprises a sequence having at least 80% sequence identity to the amino acid sequence of SEQ ID NO: 327 and the VL comprises a sequence having at least 80% sequence identity to the amino acid sequence of SEQ ID NO: 345. In some embodiments, the VH comprises a sequence having at least 80% sequence identity to the amino acid sequence of SEQ ID NO: 328 and the VL comprises a sequence having at least 80% sequence identity to the amino acid sequence of SEQ ID NO: 346. In some embodiments, the VH comprises a sequence having at least 80% sequence identity to the amino acid sequence of SEQ ID NO: 329 and the VL comprises a sequence having at least 80% sequence identity to the amino acid sequence of SEQ ID NO: 347. In some embodiments, the VH comprises a sequence having at least 80% sequence identity to the amino acid sequence of SEQ ID NO: 330 and the VL comprises a sequence having at least 80% sequence identity to the amino acid sequence of SEQ ID NO: 348. In some embodiments, the VH comprises a sequence having at least 80% sequence identity to the amino acid sequence of SEQ ID NO: 331 and the VL comprises a sequence having at least 80% sequence identity to the amino acid sequence of SEQ ID NO: 349. In some embodiments, the VH comprises a sequence having at least 80% sequence identity to the amino acid sequence of SEQ ID NO: 332 and the VL comprises a sequence having at least 80% sequence identity to the amino acid sequence of SEQ ID NO: 350. In some embodiments, the VH comprises a sequence having at least 80% sequence identity to the amino acid sequence of SEQ ID NO: 333 and the VL comprises a sequence having at least 80% sequence identity to the amino acid sequence of SEQ ID NO: 351. In some embodiments, the VH comprises a sequence having at least 80% sequence identity to the amino acid sequence of SEQ ID NO: 334 and the VL comprises a sequence having at least 80% sequence identity to the amino acid sequence of SEQ ID NO: 352. In some embodiments, the VH comprises a sequence having at least 80% sequence identity to the amino acid sequence of SEQ ID NO: 335 and the VL comprises a sequence having at least 80% sequence identity to the amino acid sequence of SEQ ID NO: 353. In some embodiments, the VH comprises a sequence having at least 80% sequence identity to the amino acid sequence of SEQ ID NO: 336 and the VL comprises a sequence having at least 80% sequence identity to the amino acid sequence of SEQ ID NO: 354. In some embodiments, the VH comprises a sequence having at least 80% sequence identity to the amino acid sequence of SEQ ID NO: 337 and the VL comprises a sequence having at least 80% sequence identity to the amino acid sequence of SEQ ID NO: 355. In some embodiments, the VH comprises a sequence having at least 80% sequence identity to the amino acid sequence of SEQ ID NO: 338 and the VL comprises a sequence having at least 80% sequence identity to the amino acid sequence of SEQ ID NO: 356. In some embodiments, the VH comprises a sequence having at least 80% sequence identity to the amino acid sequence of SEQ ID NO: 339 and the VL comprises a sequence having at least 80% sequence identity to the amino acid sequence of SEQ ID NO: 357. In some embodiments, the VH comprises a sequence having at least 80% sequence identity to the amino acid sequence of SEQ ID NO: 340 and the VL comprises a sequence having at least 80% sequence identity to the amino acid sequence of SEQ ID NO: 358. In some embodiments, the VH comprises a sequence having at least 80% sequence identity to the amino acid sequence of SEQ ID NO: 341 and the VL comprises a sequence having at least 80% sequence identity to the amino acid sequence of SEQ ID NO: 359. In some embodiments, the VH comprises a sequence having at least 80% sequence identity to the amino acid sequence of SEQ ID NO: 342 and the VL comprises a sequence having at least 80% sequence identity to the amino acid sequence of SEQ ID NO: 360. In some embodiments, the Fc is an IgG1, IgG2 or IgG4 immunoglobulin Fc domain. In some embodiments, the Fc is an IgG1 immunoglobulin domain. In some embodiments, the Fc is an IgG2 immunoglobulin domain. In some embodiments, the Fc is an IgG4 immunoglobulin domain.

Described herein, in certain embodiments, are TL1A binding proteins comprising: a) a heavy chain variable region (VH) comprising (i) a CDR1 having an amino acid sequence according to any one of SEQ ID NOs: 1-18, (ii) a CDR2 having an amino acid sequence according to any one of SEQ ID NOs: 19-36, and (iii) a CDR3 having an amino acid sequence according to any one of SEQ ID NOs: 37-54; b) a light chain variable region (VL) comprising (i) a CDR1 having an amino acid sequence according to any one of SEQ ID NOs: 55-72, (ii) a CDR2 having an amino acid sequence according to any one of SEQ ID NOs: 73-90, and (iii) a CDR3 having an amino acid sequence according to any one of SEQ ID NOs: 91-108; and c) a modified Fc that extends half-life of the TL1A binding protein as compared to a TL1A binding protein that does not comprise the modified Fc.

Described herein, in certain embodiments, are TL1A binding proteins, wherein the TL1A binding protein specifically binds to an epitope of TL1A and comprises a Fc domain comprising amino acid modifications M252Y, S254T, and T256E (YTE) and/or M428L and N434S (LS).

Described herein, in certain embodiments, are methods of treating an inflammatory bowel disease in a patient in need thereof, the method comprising subcutaneously or intravenously administering to the patient an effective amount of an TL1A binding protein described herein. In some embodiments, the inflammatory bowel disease is Crohn's disease or ulcerative colitis. In some embodiments, the inflammatory bowel disease is ulcerative colitis. In some embodiments, administration of the TL1A binding protein is subcutaneous. In some embodiments, administration of the TL1A binding protein is intravenous.

### DETAILED DESCRIPTION

To facilitate an understanding of the present disclosure, a number of terms and phrases are defined below.

As used herein, unless otherwise indicated, the term “antibody” is understood to mean an intact antibody (e.g., an intact monoclonal antibody), or a fragment thereof, such as a Fc fragment of an antibody (e.g., an Fc fragment of a monoclonal antibody), or an antigen-binding fragment of an antibody (e.g., an antigen-binding fragment of a monoclonal antibody), including an intact antibody, antigen-binding fragment, or Fc fragment that has been modified, engineered, or chemically conjugated. In general, antibodies are multimeric proteins that contain four polypeptide chains. Two of the polypeptide chains are called immunoglobulin heavy chains (H chains), and two of the polypeptide chains are called immunoglobulin light chains (L chains). The immunoglobulin heavy and light chains are connected by an interchain disulfide bond. The immunoglobulin heavy chains are connected by interchain disulfide bonds. A light chain consists of one variable region (VL) and one constant region (CL). The heavy chain consists of one variable region (VH) and at least three constant regions (CH1, CH2 and CH3). The variable regions determine the binding specificity of the antibody. Each variable region contains three hypervariable regions known as complementarity determining regions (CDRs) flanked by four relatively conserved regions known as framework regions (FRs). The extent of the FRs and CDRs has been defined (Kabat, E. A., et al. (1991) Sequences of Proteins of Immunological Interest, Fifth Edition, U.S. Department of Health and Human Services, NIH Publication No. 91-3242; and Chothia, C. et al. (1987) J. Mol. Biol. 196:901-917). The three CDRs, referred to as CDR1, CDR2, and CDR3, contribute to the antibody binding specificity. Naturally occurring antibodies have been used as starting material for engineered antibodies, such as chimeric antibodies and humanized antibodies. Examples of antibody-based antigen-binding fragments include Fab, Fab′, (Fab′)2, Fv, single chain antibodies (e.g., scFv), minibodies, and diabodies. Examples of antibodies that have been modified or engineered include chimeric antibodies, humanized antibodies, and multispecific antibodies (e.g., bispecific antibodies). An example of a chemically conjugated antibody is an antibody conjugated to a toxin moiety.

The terms “variable domain” and “variable region” are used interchangeably and refer to the portions of the antibody or immunoglobulin domains that exhibit variability in their sequence and that are involved in determining the specificity and binding affinity of a particular antibody. Variability is not evenly distributed throughout the variable domains of antibodies; it is concentrated in sub-domains of each of the heavy and light chain variable regions. These sub-domains are called “hypervariable regions” or “complementarity determining regions” (CDRs). The more conserved (i.e., non-hypervariable) portions of the variable domains are called the “framework” regions (FRM or FR) and provide a scaffold for the six CDRs in three-dimensional space to form an antigen-binding surface.

An “Fc polypeptide” of a dimeric Fc as used herein refers to one of the two polypeptides forming the dimeric Fc domain, i.e., a polypeptide comprising C-terminal constant regions of an immunoglobulin heavy chain, capable of stable self-association. For example, an Fc polypeptide of a dimeric IgG Fc comprises an IgG CH2 and an IgG CH3 constant domain sequence. An Fc can be of the class IgA, IgD, IgE, IgG, and IgM. These classes are also designated α, δ, ε, γ, and μ, respectively. Several of these may be further divided into subclasses (isotypes), e.g., IgG1, IgG2, IgG3, IgG4, IgA1, and IgA2.

The terms “Fc receptor” and “FcR” are used to describe a receptor that binds to the Fc region of an antibody. For example, an FcR can be a native sequence human FcR. Generally, an FcR is one which binds an IgG antibody (a gamma receptor) and includes receptors of the FcγRI, FcγRII, and FcγRII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Immunoglobulins of other isotypes can also be bound by certain FcRs (see, e.g., Janeway et al., Immuno Biology: the immune system in health and disease, (Elsevier Science Ltd., NY) (4th ed., 1999)). Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (reviewed in Daeron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976); and Kim et al., J. Immunol. 24:249 (1994)).

The terms “recipient,” “individual,” “subject,” “host,” and “patient,” are used interchangeably herein and in some embodiments, refer to any mammalian subject for whom diagnosis, treatment, or therapy is desired, particularly humans. “Mammal” for purposes of treatment refers to any animal classified as a mammal, including humans, domestic and farm animals, and laboratory, zoo, sports, or pet animals, such as dogs, horses, cats, cows, sheep, goats, pigs, mice, rats, rabbits, guinea pigs, monkeys etc. In some embodiments, the mammal is human. None of these terms require the supervision of medical personnel.

As used herein, the term “effective amount” refers to the amount of a compound (e.g., a compound of the present disclosure) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating, or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers, and adjuvants, see e.g., Martin, Remington's Pharmaceutical Sciences, 15th Ed., Mack Publ. Co., Easton, PA (1975).

The terms “a” and “an” as used herein mean “one or more” and include the plural unless the context is inappropriate.

As used herein, all numerical values or numerical ranges include whole integers within or encompassing such ranges and fractions of the values or the integers within or encompassing ranges unless the context clearly indicates otherwise. Thus, for example, reference to a range of 90-100%, includes 91%, 92%, 93%, 94%, 95%, 95%, 96%, 97%, etc., as well as 91.1%, 91.2%, 91.3%, 91.4%, 91.5%, etc., 92.1%, 92.2%, 92.3%, 92.4%, 92.5%, etc., and so forth. In another example, reference to a range of 1-5,000-fold includes 1, 2, 3, 4, 5, 6, 7, 8, 9, 10, 11, 12, 13, 14, 15, 16, 17, 18, 19, 20, fold, etc., as well as 1.1, 1.2, 1.3, 1.4, 1.5, fold, etc., 2.1, 2.2, 2.3, 2.4, 2.5, fold, etc., and so forth.

“About” a number, as used herein, refers to range including the number and ranging from 10% below that number to 10% above that number. “About” a range refers to 10% below the lower limit of the range, spanning to 10% above the upper limit of the range.

“Percent (%) identity” refers to the extent to which two sequences (nucleotide or amino acid) have the same residue at the same positions in an alignment. For example, “an amino acid sequence is X % identical to SEQ ID NO: Y” refers to % identity of the amino acid sequence to SEQ ID NO: Y and is elaborated as X % of residues in the amino acid sequence are identical to the residues of sequence disclosed in SEQ ID NO: Y. Generally, computer programs are employed for such calculations. Exemplary programs that compare and align pairs of sequences include ALIGN (Myers and Miller, 1988), FASTA (Pearson and Lipman, 1988; Pearson, 1990) and gapped BLAST (Altschul et al., 1997), BLASTP, BLASTN, or GCG (Devereux et al., 1984).

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

As a general matter, compositions specifying a percentage are by weight unless otherwise specified. Further, if a variable is not accompanied by a definition, then the previous definition of the variable controls.

### TL1A Binding Proteins

Provided herein are TL1A binding proteins comprising a modified Fc region. Described herein, in certain embodiments, are TL1A binding proteins comprising a) a heavy chain variable region (VH) comprising (i) a CDR1 having an amino acid sequence according to any one of SEQ ID NOs: 1-18, (ii) a CDR2 having an amino acid sequence according to any one of SEQ ID NOs: 19-36, and (iii) a CDR3 having an amino acid sequence according to any one of SEQ ID NOs: 37-54; b) a light chain variable region (VL) comprising (i) a CDR1 having an amino acid sequence according to any one of SEQ ID NOs: 55-72, (ii) a CDR2 having an amino acid sequence according to any one of SEQ ID NOs: 73-90, and (iii) a CDR3 having an amino acid sequence according to any one of SEQ ID NOs: 91-108; and c) a Fc domain comprising amino acid modifications M252Y, S254T, and T256E (YTE) and/or M428L and N434S (LS).

Further described herein, in certain embodiments, are TL1A binding proteins comprising: a) a heavy chain variable region (VH) comprising (i) a CDR1 having an amino acid sequence according to any one of SEQ ID NOs: 1-18, (ii) a CDR2 having an amino acid sequence according to any one of SEQ ID NOs: 19-36, and (iii) a CDR3 having an amino acid sequence according to any one of SEQ ID NOs: 37-54; b) a light chain variable region (VL) comprising (i) a CDR1 having an amino acid sequence according to any one of SEQ ID NOs: 55-72, (ii) a CDR2 having an amino acid sequence according to any one of SEQ ID NOs: 73-90, and (iii) a CDR3 having an amino acid sequence according to any one of SEQ ID NOs: 91-108; and c) a modified Fc that extends half-life of the TL1A binding protein as compared to a TL1A binding protein that does not comprise the modified Fc.

Further described herein, in certain embodiments, are TL1A binding proteins, wherein the TL1A binding protein specifically binds to an epitope of TL1A and comprises a Fc domain comprising amino acid modifications M252Y, S254T, and T256E (YTE) and/or M428L and N434S (LS).

Amino acid sequences of exemplary CDRs of TL1A binding proteins are provided in Table 1.

TABLE 1
Sequences of CDRs

| | | | | | | | | | | | | |
|---|---|---|---|---|---|---|---|---|---|---|---|---|
| | SEQ ID | | SEQ ID | | SEQ | | SEQ | | SEQ ID | | SEQ | |
| Antibody | NO | CDRH1 | NO | CDRH2 | ID NO | CDRH3 | ID NO | CDRL1 | NO | CDRL2 | ID NO | CDRL3 |

Kabat Numbering

| | | | | | | | | | | | | |
|---|---|---|---|---|---|---|---|---|---|---|---|---|
| Antibody 1 | 1 | YYGIS | 19 | WISTYNGNTH | 37 | ENYYGSGA | 55 | RASQSVS | 73 | DASNRAT | 91 | QQRSNWPW |
| | | | | YARMLQG | | YRGGMDV | | SYLA | | | | T |
| Antibody 2 | 2 | DTYMH | 20 | RIDPASGHTK | 38 | SGGLPDV | 56 | RASSSVS | 74 | ATSNLAS | 92 | QQWEGNPR |
| | | | | YDPKFQV | | | | YMY | | | | T |
| Antibody 3 | 3 | GYYWN | 21 | EINHAGNTNY | 39 | GYCRSTTCY | 57 | RASQSVR | 75 | GASSRAT | 93 | QQYGSSPT |
| | | | | NPSLKS | | FDY | | SSYLA | | | | |
| Antibody 4 | 4 | SYDIN | 22 | WLNPNSGYTG | 40 | EVPETAAFE | 58 | TSSSSDIG | 76 | GYYNRPS | 94 | QSWDGTLS |
| | | | | YAQKFQG | | Y | | AGLGVH | | | | AL |
| Antibody 5 | 5 | SYWMS | 23 | NIKEDGSEKN | 41 | EDYDSYYK | 59 | KSSQSIL | 77 | WASTRES | 95 | QQYYSTPFT |
| | | | | YVDSVKG | | YGMDV | | YSSNNK | | | | |
| | | | | | | | | NYLA | | | | |
| Antibody 6 | 6 | GYYMH | 24 | WIKPKSGGTN | 42 | GGSQDASDF | 60 | RASQDISI | 78 | AASSLOS | 96 | QQADSFPLT |
| | | | | YVQKFQG | | | | WLA | | | | |
| Antibody 7 | 7 | DYHWA | 25 | DINHRGRTNY | 43 | DFPNWVFDS | 61 | RASQSVS | 79 | GASRRAT | 97 | QQYGSSPLT |
| | | | | NPSLKS | | | | SSFLA | | | | |
| Antibody 8 | 8 | GFYWS | 26 | EINHRGNTNY | 44 | PFYDFWSGS | 62 | KSSQSLV | 80 | KISNRFS | 98 | MQATQFPLT |
| | | | | NPSLKS | | DY | | HSDGNT | | | | |
| | | | | | | | | YLS | | | | |
| Antibody 9 | 9 | DYFWT | 27 | EISHSGRTNYN | 45 | DYPNWVFD | 63 | RASQSVF | 81 | GASRRAT | 99 | QQYGNSPLT |
| | | | | PSLKS | | Y | | NSYLA | | | | |
| Antibody | 10 | DYYMS | 28 | YIGGSGSTIYY | 46 | EDADSYYY | 64 | KSSQSVL | 82 | WASTRES | 100 | QQYYSTPRT |
| 10 | | | | ADSVKG | | YGMDV | | YSSNNK | | | | |
| | | | | | | | | NHLS | | | | |
| Antibody | 11 | SSWMH | 29 | EIHPNSGGTNY | 47 | GDYYGYVS | 65 | QASQNIN | 83 | KASNLHT | 101 | QQGQSYPY |
| 11 | | | | AQKFQG | | WFAY | | VLLN | | | | T |
| Antibody | 12 | SYDIN | 30 | WLNPNSGNTG | 48 | EVPETAAFE | 66 | TSSSSDIG | 84 | GYYNRPS | 102 | QSYDGTLSA |
| 12 | | | | YAQKFQG | | Y | | AGLGVH | | | | L |
| Antibody | 13 | DTYMH | 31 | RIDPASGHTK | 49 | SGGLPDV | 67 | RASSSVS | 85 | ATSNLAS | 103 | QQWSGNPR |
| 13 | | | | YDPKFQV | | | | YMY | | | | T |
| Antibody | 14 | DTYMH | 32 | RIDPASGHTK | 50 | SGGLPDV | 68 | RASSSVS | 86 | ATSNLAS | 104 | QQWSGNPR |
| 14 | | | | YDPKFQV | | | | YMY | | | | T |
| Antibody | 15 | DTYMH | 33 | RIDPASGHTK | 51 | SGGLPDV | 69 | RASSSVS | 87 | ATSNLAS | 105 | QQWSGNPR |
| 15 | | | | YDPKFQV | | | | YMY | | | | T |
| Antibody | 16 | DTYMH | 34 | RIDPASGHTK | 52 | SGGLPDV | 70 | RASSSVS | 88 | ATSNLAS | 106 | QQWSGNPR |
| 16 | | | | YDPKFQV | | | | YMY | | | | T |
| Antibody | 17 | TYGMS | 35 | WMNTYSGVT | 53 | EGYVFDDY | 71 | RSSQNIV | 89 | KVSNRFS | 107 | FQGSHVPLT |
| 17 | | | | TYADDFKG | | YATDY | | HSDGNT | | | | |
| | | | | | | | | YLE | | | | |
| Antibody | 18 | SRSYYW | 36 | SIYYNGRTYY | 54 | EDYGDYGA | 72 | RASQGIS | 90 | DASSLES | 108 | QQFNSYPLT |
| 18 | | G | | NPSLKS | | FDI | | SALA | | | | |
| Antibody 1 | 109 | GYDFTY | 127 | STYNGN | 145 | NYYGSGAY | 163 | SQSVSSY | None | DAS | 199 | RSNWPW |
| | | Y | | | | RGGMD | | | | | | |
| Antibody 2 | 110 | GFDIQDT | 128 | DPASGH | 146 | GGLPD | 164 | SSSVSY | None | ATS | 200 | WEGNPR |
| Antibody 3 | 111 | GGSFSGY | 129 | NHAGN | 147 | YCRSTTCYF | 165 | SQSVRSS | None | GAS | 201 | YGSSP |
| | | | | | | D | | Y | | | | |
| Antibody 4 | 112 | GYTFTSY | 130 | NPNSGY | 148 | VPETAAFE | 166 | SSSSDIG | None | GYY | 202 | WDGTLSA |
| | | | | | | | | AGLG | | | | |
| Antibody 5 | 113 | GFTFSSY | 131 | KEDGSE | 149 | DYDSYYKY | 167 | SQSILYSS | None | WAS | 203 | YYSTPF |
| | | | | | | GMD | | NNKNY | | | | |
| Antibody 6 | 114 | GYTFTGY | 132 | KPKSGG | 150 | GSQDASD | 168 | SQDISIW | None | AAS | 204 | ADSFPL |
| Antibody 7 | 115 | GVSFSDY | 133 | NHRGR | 151 | FPNWVFD | 169 | SQSVSSS | None | GAS | 205 | YGSSPL |
| | | | | | | | | F | | | | |
| Antibody 8 | 116 | GGSFTGF | 134 | NHRGN | 152 | FYDFWSGSD | 170 | SQSL VHS | None | KIS | 206 | ATQFPL |
| | | | | | | | | DGNTY | | | | |
| Antibody 9 | 117 | GGSFSDY | 135 | SHSGR | 153 | YPNWVFD | 171 | SQSVENS | None | GAS | 207 | YGNSPL |
| | | | | | | | | Y | | | | |
| Antibody | 118 | GFTISDY | 136 | GGSGST | 154 | DADSYYYY | 172 | SQSVLYS | None | WAS | 208 | YYSTPR |
| 10 | | | | | | GMD | | SNNKNH | | | | |
| Antibody | 119 | GYTFTSS | 137 | HPNSGG | 155 | DYYGYVSW | 173 | SQNINVL | None | KAS | 209 | GQSYPY |
| 11 | | | | | | FA | | | | | | |
| Antibody | 120 | GYTFTSY | 138 | NPNSGN | 156 | VPETAAFE | 174 | SSSSDIG | None | GYY | 210 | YDGTLSA |
| 12 | | | | | | | | AGLG | | | | |
| Antibody | 121 | GFDICDT | 139 | DPASGH | 157 | GGLPD | 175 | SSSVSY | None | ATS | 211 | WSGNPR |
| 13 | | | | | | | | | | | | |
| Antibody | 122 | GFDICDT | 140 | DPASGH | 158 | GGLPD | 176 | SSSVSY | None | ATS | 212 | WSGNPR |
| 14 | | | | | | | | | | | | |
| Antibody | 123 | GFDICDT | 141 | DPASGH | 159 | GGLPD | 177 | SSSVSY | None | ATS | 213 | WSGNPR |
| 15 | | | | | | | | | | | | |
| Antibody | 124 | GFDICDT | 142 | DPASGH | 160 | GGLPD | 178 | SSSVSY | None | ATS | 214 | WSGNPR |
| 16 | | | | | | | | | | | | |
| Antibody | 125 | GYTFTTY | 143 | NTYSGV | 161 | GYVFDDYY | 179 | SQNIVHS | None | KVS | 215 | GSHVPL |
| 17 | | | | | | ATD | | DGNTY | | | | |
| Antibody | 126 | GGSISSRS | 144 | YYNGR | 162 | EDYGDYGA | 180 | RASQGIS | 198 | DASSLES | 216 | QQFNSYPLT |
| 18 | | Y | | | | FDI | | SALA | | | | |

IMGT Numbering

| | | | | | | | | | | | | |
|---|---|---|---|---|---|---|---|---|---|---|---|---|
| Antibody 1 | 217 | GYDFTY | 235 | ISTYNGNT | 253 | ARENYYGSG | 271 | QSVSSY | None | DAS | 307 | QQRSNWPW |
| | | YG | | | | AYRGGMDV | | | | | | T |
| Antibody 2 | 218 | GFDIQDT | 236 | IDPASGHT | 254 | ARSGGLPDV | 272 | SSVSY | None | ATS | 308 | QQWEGNPR |
| | | Y | | | | | | | | | | T |
| Antibody 3 | 219 | GGSFSGY | 237 | INHAGNI | 255 | ARGYCRSTT | 273 | QSVRSSY | None | GAS | 309 | QQYGSSPT |
| | | Y | | | | CYFDY | | | | | | |
| Antibody 4 | 220 | GYTFTSY | 238 | LNPNSGYT | 256 | AREVPETAA | 274 | SSDIGAG | None | GYY | 310 | QSWDGTLS |
| | | D | | | | FEY | | LG | | | | AL |
| Antibody 5 | 221 | GFTFSSY | 239 | IKEDGSEK | 257 | AREDYDSYY | 275 | QSIL YSS | None | WAS | 311 | QQYYSTPFT |
| | | W | | | | KYGMDV | | NNKNY | | | | |
| Antibody 6 | 222 | GYTFTGY | 240 | IKPKSGGT | 258 | ATGGSQDAS | 276 | QDISIW | None | AAS | 312 | QQADSFPLT |
| | | Y | | | | DF | | | | | | |
| Antibody 7 | 223 | GVSFSDY | 241 | INHRGRT | 259 | ARDFPNWVF | 277 | QSVSSSF | None | GAS | 313 | QQYGSSPLT |
| | | H | | | | DS | | | | | | |
| Antibody 8 | 224 | GGSFTGF | 242 | INHRGNT | 260 | ASPFYDFWS | 278 | QSL VHSD | None | KIS | 314 | MQATQFPLT |
| | | Y | | | | GSDY | | GNTY | | | | |
| Antibody 9 | 225 | GGSFSDY | 243 | ISHSGRT | 261 | ARDYPNWV | 279 | QSVFNSY | None | GAS | 315 | QQYGNSPLT |
| | | F | | | | FDY | | | | | | |
| Antibody | 226 | GFTISDY | 244 | IGGSGSTI | 262 | AREDADSYY | 280 | QSVLYSS | None | WAS | 316 | QQYYSTPRT |
| 10 | | Y | | | | YYGMDV | | NNKNH | | | | |
| Antibody | 227 | GYTFTSS | 245 | IHPNSGGT | 263 | ARGDYYGY | 281 | QNINVL | None | KAS | 317 | QQGQSYPY |
| 11 | | W | | | | VSWFAY | | | | | | T |
| Antibody | 228 | GYTFTSY | 246 | LNPNSGNT | 264 | AREVPETAA | 282 | SSDIGAG | None | GYY | 318 | QSYDGTLSA |
| 12 | | D | | | | FEY | | LG | | | | L |
| Antibody | 229 | GFDICDT | 247 | IDPASGHT | 265 | SRSGGLPDV | 283 | SSVSY | None | ATS | 319 | QQWSGNPR |
| 13 | | Y | | | | | | | | | | T |
| Antibody | 230 | GFDICDT | 248 | IDPASGHT | 266 | ARSGGLPDV | 284 | SSVSY | None | ATS | 320 | QQWSGNPR |
| 14 | | Y | | | | | | | | | | T |
| Antibody | 231 | GFDICDT | 249 | IDPASGHT | 267 | ARSGGLPDV | 285 | SSVSY | None | ATS | 321 | QQWSGNPR |
| 15 | | Y | | | | | | | | | | T |
| Antibody | 232 | GFDICDT | 250 | IDPASGHT | 268 | ARSGGLPDV | 286 | SSVSY | None | ATS | 322 | QQWSGNPR |
| 16 | | Y | | | | | | | | | | T |
| Antibody | 233 | GYTFTTY | 251 | MNTYSGVT | 269 | AREGYVFDD | 287 | QNIVHSD | None | KVS | 323 | FQGSHVPLT |
| 17 | | G | | | | YYATDY | | GNTY | | | | |
| Antibody | 234 | GGSISSRS | 252 | IYYNGRT | 270 | AREDYGDY | 288 | QGISSA | None | DA | 324 | QQFNSYPLT |
| 18 | | YY | | | | GAFDI | | | | | | |

In some embodiments, the TL1A binding protein comprises a heavy chain variable region comprising a CDR1, CDR2, and CDR3 as listed in Table 1. In some embodiments, the TL1A binding protein comprises a heavy chain variable region comprising (a) a CDR1 having an amino acid sequence according to any one of SEQ ID NOs: 1-18, (b) a CDR2 having an amino acid sequence according to any one of SEQ ID NOs: 19-36, and (c) a CDR3 having an amino acid sequence according to any one of SEQ ID NOs: 37-54.

In some embodiments, the TL1A binding protein comprises a light chain variable region comprising a CDR1, CDR2, and CDR3 as listed in Table 1. In some embodiments, the TL1A binding protein comprises a light chain variable region comprising (a) a CDR1 having an amino acid sequence according to any one of SEQ ID NOs: 55-72, (b) a CDR2 having an amino acid sequence according to any one of SEQ ID NOs: 73-90, and (c) a CDR3 having an amino acid sequence according to any one of SEQ ID NOs: 91-108.

In some embodiments, the TL1A binding protein comprises a heavy chain variable region comprising (a) a CDR1 having an amino acid sequence according to any one of SEQ ID NOs: 1-18, (b) a CDR2 having an amino acid sequence according to any one of SEQ ID NOs: 19-36, and (c) a CDR3 having an amino acid sequence according to any one of SEQ ID NOs: 37-54; and a light chain variable region comprising (a) a CDR1 having an amino acid sequence according to any one of SEQ ID NOs: 55-72, (b) a CDR2 having an amino acid sequence according to any one of SEQ ID NOs: 73-90, and (c) a CDR3 having an amino acid sequence according to any one of SEQ ID NOs: 91-108.

In some embodiments, the TL1A binding protein comprises a heavy chain variable region comprising (a) a CDR1 having an amino acid sequence according to any one of SEQ ID NOs: 109-126, (b) a CDR2 having an amino acid sequence according to any one of SEQ ID NOs: 127-144, and (c) a CDR3 having an amino acid sequence according to any one of SEQ ID NOs: 145-162.

In some embodiments, the TL1A binding protein comprises a light chain variable region comprising (a) a CDR1 having an amino acid sequence according to any one of SEQ ID NOs: 163-180, (b) a CDR2 having an amino acid sequence according to DAS, ATS, GAS, GYY, WAS, AAS, KIS, KAS or KVS or SEQ ID NO: 198, and (c) a CDR3 having an amino acid sequence according to any one of SEQ ID NOs: 199-216.

In some embodiments, the TL1A binding protein comprises a heavy chain variable region comprising (a) a CDR1 having an amino acid sequence according to any one of SEQ ID NOs: 109-126, (b) a CDR2 having an amino acid sequence according to any one of SEQ ID NOs: 127-144, and (c) a CDR3 having an amino acid sequence according to any one of SEQ ID NOs: 145-162; and a light chain variable region comprising (a) a CDR1 having an amino acid sequence according to any one of SEQ ID NOs: 163-180, (b) a CDR2 having an amino acid sequence according to any one of DAS, ATS, GAS, GYY, WAS, AAS, KIS, KAS or KVS or SEQ ID NO: 198, and (c) a CDR3 having an amino acid sequence according to any one of SEQ ID NOs: 199-216.

In some embodiments, the TL1A binding protein comprises a heavy chain variable region comprising (a) a CDR1 having an amino acid sequence according to any one of SEQ ID NOs: 217-234, (b) a CDR2 having an amino acid sequence according to any one of SEQ ID NOs: 235-252, and (c) a CDR3 having an amino acid sequence according to any one of SEQ ID NOs: 253-270.

In some embodiments, the TL1A binding protein comprises a light chain variable region comprising (a) a CDR1 having an amino acid sequence according to any one of SEQ ID NOs: 271-288, (b) a CDR2 having an amino acid sequence according to any one of DAS, ATS, GAS, GYY, WAS, AAS, KIS, KAS, KVS or DA, and (c) a CDR3 having an amino acid sequence according to any one of SEQ ID NOs: 307-324.

In some embodiments, the TL1A binding protein comprises a heavy chain variable region comprising (a) a CDR1 having an amino acid sequence according to any one of SEQ ID NOs: 217-234, (b) a CDR2 having an amino acid sequence according to any one of SEQ ID NOs: 235-252, and (c) a CDR3 having an amino acid sequence according to any one of SEQ ID NOs: 253-270; and a light chain variable region comprising (a) a CDR1 having an amino acid sequence according to any one of SEQ ID NOs: 271-288, (b) a CDR2 having an amino acid sequence according to any one of DAS, ATS, GAS, GYY, WAS, AAS, KIS, KAS, KVS or DA, and (c) a CDR3 having an amino acid sequence according to any one of SEQ ID NOs: 307-324.

In some embodiments, the TL1A binding protein comprises a heavy chain variable region comprising (a) a CDR1 having an amino acid sequence of SEQ ID NO: 1, (b) a CDR2 having an amino acid sequence of SEQ ID NO: 19, and (c) a CDR3 having an amino acid sequence of SEQ ID NO: 37; and a light chain variable region comprising (a) a CDR1 having an amino acid sequence of SEQ ID NO: 55, (b) a CDR2 having an amino acid sequence of SEQ ID NO: 73, and (c) a CDR3 having an amino acid sequence of SEQ ID NO: 91.

In some embodiments, the TL1A binding protein comprises a heavy chain variable region comprising (a) a CDR1 having an amino acid sequence of SEQ ID NO: 2, (b) a CDR2 having an amino acid sequence of SEQ ID NO: 20, and (c) a CDR3 having an amino acid sequence of SEQ ID NO: 38; and a light chain variable region comprising (a) a CDR1 having an amino acid sequence of SEQ ID NO: 56, (b) a CDR2 having an amino acid sequence of SEQ ID NO: 74, and (c) a CDR3 having an amino acid sequence of SEQ ID NO: 92.

In some embodiments, the TL1A binding protein comprises a heavy chain variable region comprising (a) a CDR1 having an amino acid sequence of SEQ ID NO: 3, (b) a CDR2 having an amino acid sequence of SEQ ID NO: 21, and (c) a CDR3 having an amino acid sequence of SEQ ID NO: 39; and a light chain variable region comprising (a) a CDR1 having an amino acid sequence of SEQ ID NO: 57, (b) a CDR2 having an amino acid sequence of SEQ ID NO: 75, and (c) a CDR3 having an amino acid sequence of SEQ ID NO: 93.

In some embodiments, the TL1A binding protein comprises a heavy chain variable region comprising (a) a CDR1 having an amino acid sequence of SEQ ID NO: 4, (b) a CDR2 having an amino acid sequence of SEQ ID NO: 22, and (c) a CDR3 having an amino acid sequence of SEQ ID NO: 40; and a light chain variable region comprising (a) a CDR1 having an amino acid sequence of SEQ ID NO: 58, (b) a CDR2 having an amino acid sequence of SEQ ID NO: 76, and (c) a CDR3 having an amino acid sequence of SEQ ID NO: 94.

In some embodiments, the TL1A binding protein comprises a heavy chain variable region comprising (a) a CDR1 having an amino acid sequence of SEQ ID NO: 5, (b) a CDR2 having an amino acid sequence of SEQ ID NO: 23, and (c) a CDR3 having an amino acid sequence of SEQ ID NO: 41; and a light chain variable region comprising (a) a CDR1 having an amino acid sequence of SEQ ID NO: 59, (b) a CDR2 having an amino acid sequence of SEQ ID NO: 77, and (c) a CDR3 having an amino acid sequence of SEQ ID NO: 95.

In some embodiments, the TL1A binding protein comprises a heavy chain variable region comprising (a) a CDR1 having an amino acid sequence of SEQ ID NO: 6, (b) a CDR2 having an amino acid sequence of SEQ ID NO: 24, and (c) a CDR3 having an amino acid sequence of SEQ ID NO: 42; and a light chain variable region comprising (a) a CDR1 having an amino acid sequence of SEQ ID NO: 60, (b) a CDR2 having an amino acid sequence of SEQ ID NO: 78, and (c) a CDR3 having an amino acid sequence of SEQ ID NO: 96.

In some embodiments, the TL1A binding protein comprises a heavy chain variable region comprising (a) a CDR1 having an amino acid sequence of SEQ ID NO: 7, (b) a CDR2 having an amino acid sequence of SEQ ID NO: 25, and (c) a CDR3 having an amino acid sequence of SEQ ID NO: 43; and a light chain variable region comprising (a) a CDR1 having an amino acid sequence of SEQ ID NO: 61, (b) a CDR2 having an amino acid sequence of SEQ ID NO: 79, and (c) a CDR3 having an amino acid sequence of SEQ ID NO: 97.

In some embodiments, the TL1A binding protein comprises a heavy chain variable region comprising (a) a CDR1 having an amino acid sequence of SEQ ID NO: 8, (b) a CDR2 having an amino acid sequence of SEQ ID NO: 26, and (c) a CDR3 having an amino acid sequence of SEQ ID NO: 44; and a light chain variable region comprising (a) a CDR1 having an amino acid sequence of SEQ ID NO: 62, (b) a CDR2 having an amino acid sequence of SEQ ID NO: 80, and (c) a CDR3 having an amino acid sequence of SEQ ID NO: 98.

In some embodiments, the TL1A binding protein comprises a heavy chain variable region comprising (a) a CDR1 having an amino acid sequence of SEQ ID NO: 9, (b) a CDR2 having an amino acid sequence of SEQ ID NO: 27, and (c) a CDR3 having an amino acid sequence of SEQ ID NO: 45; and a light chain variable region comprising (a) a CDR1 having an amino acid sequence of SEQ ID NO: 63, (b) a CDR2 having an amino acid sequence of SEQ ID NO: 81, and (c) a CDR3 having an amino acid sequence of SEQ ID NO: 99.

In some embodiments, the TL1A binding protein comprises a heavy chain variable region comprising (a) a CDR1 having an amino acid sequence of SEQ ID NO: 10, (b) a CDR2 having an amino acid sequence of SEQ ID NO: 28, and (c) a CDR3 having an amino acid sequence of SEQ ID NO: 46; and a light chain variable region comprising (a) a CDR1 having an amino acid sequence of SEQ ID NO: 64, (b) a CDR2 having an amino acid sequence of SEQ ID NO: 82, and (c) a CDR3 having an amino acid sequence of SEQ ID NO: 100.

In some embodiments, the TL1A binding protein comprises a heavy chain variable region comprising (a) a CDR1 having an amino acid sequence of SEQ ID NO: 11, (b) a CDR2 having an amino acid sequence of SEQ ID NO: 29, and (c) a CDR3 having an amino acid sequence of SEQ ID NO: 47; and a light chain variable region comprising (a) a CDR1 having an amino acid sequence of SEQ ID NO: 65, (b) a CDR2 having an amino acid sequence of SEQ ID NO: 83, and (c) a CDR3 having an amino acid sequence of SEQ ID NO: 101.

In some embodiments, the TL1A binding protein comprises a heavy chain variable region comprising (a) a CDR1 having an amino acid sequence of SEQ ID NO: 12, (b) a CDR2 having an amino acid sequence of SEQ ID NO: 30, and (c) a CDR3 having an amino acid sequence of SEQ ID NO: 48; and a light chain variable region comprising (a) a CDR1 having an amino acid sequence of SEQ ID NO: 66, (b) a CDR2 having an amino acid sequence of SEQ ID NO: 84, and (c) a CDR3 having an amino acid sequence of SEQ ID NO: 102.

In some embodiments, the TL1A binding protein comprises a heavy chain variable region comprising (a) a CDR1 having an amino acid sequence of SEQ ID NO: 13, (b) a CDR2 having an amino acid sequence of SEQ ID NO: 31, and (c) a CDR3 having an amino acid sequence of SEQ ID NO: 49; and a light chain variable region comprising (a) a CDR1 having an amino acid sequence of SEQ ID NO: 67, (b) a CDR2 having an amino acid sequence of SEQ ID NO: 85, and (c) a CDR3 having an amino acid sequence of SEQ ID NO: 103.

In some embodiments, the TL1A binding protein comprises a heavy chain variable region comprising (a) a CDR1 having an amino acid sequence of SEQ ID NO: 14, (b) a CDR2 having an amino acid sequence of SEQ ID NO: 32, and (c) a CDR3 having an amino acid sequence of SEQ ID NO: 50; and a light chain variable region comprising (a) a CDR1 having an amino acid sequence of SEQ ID NO: 68, (b) a CDR2 having an amino acid sequence of SEQ ID NO: 86, and (c) a CDR3 having an amino acid sequence of SEQ ID NO: 104.

In some embodiments, the TL1A binding protein comprises a heavy chain variable region comprising (a) a CDR1 having an amino acid sequence of SEQ ID NO: 15, (b) a CDR2 having an amino acid sequence of SEQ ID NO: 33, and (c) a CDR3 having an amino acid sequence of SEQ ID NO: 51; and a light chain variable region comprising (a) a CDR1 having an amino acid sequence of SEQ ID NO: 69, (b) a CDR2 having an amino acid sequence of SEQ ID NO: 87, and (c) a CDR3 having an amino acid sequence of SEQ ID NO: 105.

In some embodiments, the TL1A binding protein comprises a heavy chain variable region comprising (a) a CDR1 having an amino acid sequence of SEQ ID NO: 16, (b) a CDR2 having an amino acid sequence of SEQ ID NO: 34, and (c) a CDR3 having an amino acid sequence of SEQ ID NO: 52; and a light chain variable region comprising (a) a CDR1 having an amino acid sequence of SEQ ID NO: 70, (b) a CDR2 having an amino acid sequence of SEQ ID NO: 88, and (c) a CDR3 having an amino acid sequence of SEQ ID NO: 106.

In some embodiments, the TL1A binding protein comprises a heavy chain variable region comprising (a) a CDR1 having an amino acid sequence of SEQ ID NO: 17, (b) a CDR2 having an amino acid sequence of SEQ ID NO: 35, and (c) a CDR3 having an amino acid sequence of SEQ ID NO: 53; and a light chain variable region comprising (a) a CDR1 having an amino acid sequence of SEQ ID NO: 71, (b) a CDR2 having an amino acid sequence of SEQ ID NO: 89, and (c) a CDR3 having an amino acid sequence of SEQ ID NO: 107.

In some embodiments, the TL1A binding protein comprises a heavy chain variable region comprising (a) a CDR1 having an amino acid sequence of SEQ ID NO: 18, (b) a CDR2 having an amino acid sequence of SEQ ID NO: 36, and (c) a CDR3 having an amino acid sequence of SEQ ID NO: 54; and a light chain variable region comprising (a) a CDR1 having an amino acid sequence of SEQ ID NO: 72, (b) a CDR2 having an amino acid sequence of SEQ ID NO: 90, and (c) a CDR3 having an amino acid sequence of SEQ ID NO: 108.

Amino acid sequences of exemplary heavy chain variable regions (VH) and light chain variable regions (VL) of TL1A binding proteins are provided in Table 2.

TABLE 2
Sequences of heavy chain variable regions (VH) and
light chain variable regions (VL) of TL1A binding proteins

| | SEQ | | SEQ | |
|---|---|---|---|---|
| | ID | | ID | |
| Antibody | NO | VH | NO | VL |
| Antibody | 325 | QVQLVQSGAEVKKPGASVKVSC | 343 | EIVLTQSPATLSLSPGERAT |
| 1 | | KASGYDFTYYGISWVRQAPGQGL | | LSCRASQSVSSYLAWYQQ |
| | | EWMGWISTYNGNTHYARMLQGR | | KPGQAPRLLIYDASNRATG |
| | | VTMTTDTSTRTAYMELRSLRSDD | | IPARFSGSGSGTDFTLTISSL |
| | | TAVYYCARENYYGSGAYRGGMD | | EPEDFAVYYCQQRSNWPW |
| | | VWGQGTTVTVSS | | TFGQGTKVEIK |
| Antibody | 326 | QVQLVQSGAEVKKPGASVKVSC | 344 | EIVLTQSPGTLSLSPGERAT |
| 2 | | KASGFDIQDTYMHWVKQRPGQG | | LSCRASSSVSYMYWYQQK |
| | | LEWMGRIDPASGHTKYDPKFQVR | | PGQAPRPLIYATSNLASGIP |
| | | VTITRDTSTSTVYLELSSLRSEDTA | | DRFSGSGSGTDFTLTISRLE |
| | | VYYCARSGGLPDVWGQGTTVTV | | PEDFAVYYCQQWEGNPRT |
| | | SS | | FGGGTKLEIK |
| Antibody | 327 | QVQLQQWGAGLLKPSETLSLTCA | 345 | EIVLIQSPGILSLSPGERATL |
| 3 | | VHGGSFSGYYWNWIRQPPGKGLE | | SCRASQSVRSSYLAWYQQ |
| | | WIGEINHAGNTNYNPSLKSRVTIS | | KPGQAPRLLIYGASSRATG |
| | | LDTSKNQFSLTLTSVTAADTAVY | | IPDRFSGSGSGTDFTLTISR |
| | | YCARGYCRSTTCYFDYWGQGTL | | LEPEDFAVYYCQQYGSSPT |
| | | VTVSS | | FGQGTRLEIK |
| Antibody | 328 | QVQLVQSGAEVKKPGASVKVSC | 346 | QSVLTQPPSVSGAPGQRVT |
| 4 | | KASGYTFTSYDINWVRQAPGQGL | | ISCTSSSSDIGAGLGVHWY |
| | | EWMGWLNPNSGYTGYAQKFQG | | QQLPGTAPKLLIEGYYNRP |
| | | RVTMTADRSTSTAYMELSSLRSE | | SGVPDRFSGTSASLTITGLL |
| | | DTAVYYCAREVPETAAFEYWGQ | | PEDEGDYYCQSWDGTLSA |
| | | GTLVTVSS | | LFGGGTKLTVL |
| Antibody | 329 | EVQLVESGGGLVQPGGSLRLSCA | 347 | DIVMTQSPDSLAVSLGERA |
| 5 | | VSGFTFSSYWMSWVRQAPGKGL | | TINCKSSQSILYSSNNKNYL |
| | | EWVANIKEDGSEKNYVDSVKGRF | | AWYQQKPGQPPKLLIYWA |
| | | TLSSDNAKNSLYLQMNSLRAEDT | | STRESGVPDRFSGSGSGTD |
| | | AVYYCAREDYDSYYKYGMDVW | | FTLTISSLQAEDVSVYYCQ |
| | | GQGTAVIVSS | | QYYSTPFTFGPGTKVDIK |
| Antibody | 330 | QVQLVESGAEVKKPGASVKVSCK | 348 | DIQLTQSPSSVSASVGGRV |
| 6 | | ASGYTFTGYYMHWVRQAPGQGL | | TITCRASQDISIWLAWYQQ |
| | | EWMGWIKPKSGGTNYVQKFQGR | | KPGKAPNLLIYAASSLQSG |
| | | VTMTRDTSINTAYMELNNLKSDD | | VPSRFSGSGSGTDFTLTISS |
| | | TAVYYCATGGSQDASDFWGQGT | | LQPEDFATYYCQQADSFPL |
| | | MVTVSS | | TFGGGTKLEIKR |
| Antibody | 331 | QVQLHQWGAGLLKPSETLSLTCA | 349 | EIVLSQSPGTLSLSPGERAT |
| 7 | | VSGVSFSDYHWAWIRQSPGKGLE | | LSCRASQSVSSSFLAWYQQ |
| | | WIGDINHRGRTNYNPSLKSRVTIS | | YPGQAPRLLIYGASRRATG |
| | | LDTSGNPFSLKLTSVTAADTAVY | | IPDRFRGSGSGTDFTLTISR |
| | | YCARDFPNWVFDSWGQGILVTVS | | LEPEDFAVYYCQQYGSSPL |
| | | S | | TFGGGTKVENK |
| Antibody | 332 | QVQLQQWGAGLLKPSETLSLTCA | 350 | DIMLTQTPLTSPVTLGQPA |
| 8 | | VYGGSFTGFYWSWIRQPPGKGLE | | SISCKSSQSLVHSDGNTYLS |
| | | WIGEINHRGNTNYNPSLKSRVTM | | WLQQRPGQPPRLLFYKISN |
| | | SVDTSKNQFSLNMISVTAADTAM | | RFSGVPDRFSGSGAGTDFT |
| | | YFCASPFYDFWSGSDYWGQGTL | | LKISRVEAEDVGVYYCMQ |
| | | VTVSS | | ATQFPLTFGGGTKVEIK |
| Antibody | 333 | QVQLQQWGAGLLKPSETLSLTCA | 351 | EIELTQSPGTLSLSPGEGAT |
| 9 | | VSGGSFSDYFWTWIRQPPGKGLE | | LSCRASQSVFNSYLAWYQ |
| | | WIGEISHSGRTNYNPSLKSRVTISV | | QKPGQAPRLLIYGASRRAT |
| | | DTSMSQFSLKMTSVTAADSAVYY | | GIPDRFSGSGSGTDFTLTIS |
| | | CARDYPNWVFDYWGQGTLVTVS | | RLEPGDFAVYFCQQYGNS |
| | | S | | PLTFGGGTKVEIK |
| Antibody | 334 | RLQLVESGGGLVKPGGSLKLSCA | 352 | DIVMTQSPDSLAVSLGERA |
| 10 | | ASGFTISDYYMSWIRQAPGKGLE | | TINCKSSQSVLYSSNNKNH |
| | | WVSYIGGSGSTIYYADSVKGRFTI | | LSWYQQKPGQPPKLLIYW |
| | | SRDNAKNSLYLQMNSLRAEDTA | | ASTRESGVPDRFSGSGSGT |
| | | VYYCAREDADSYYYYGMDVWG | | DFTLTISSLQAEDVAVYYC |
| | | QGTTVTVSS | | QQYYSTPRTFGQGTKVEIK |
| Antibody | 335 | QVQLVQSGAEVKKPGASVKVSC | 353 | DIQMTQSPSSLSASVGDRV |
| 11 | | KASGYTFTSSWMHWARQAPGQG | | TITCQASQNINVLLNWYQQ |
| | | LEWMGEIHPNSGGTNYAQKFQG | | KPGKAPKLLIYKASNLHTG |
| | | RVTMTVDTSISTAYMELSRLRSD | | VPSRFSGSGSGTDFTFTISS |
| | | DTAVYYCARGDYYGYVSWFAY | | LQPEDIATYYCQQGQSYPY |
| | | WGQGTLVTVSS | | TFGQGTKLEIK |
| Antibody | 336 | QVQLVQSGAEVKKPGASVKVSC | 354 | QSVLTQPPSVSGAPGQRVT |
| 12 | | KASGYTFTSYDINWVRQAPGQGL | | ISCTSSSSDIGAGLGVHWY |
| | | EWMGWLNPNSGNTGYAQKFQG | | QQLPGTAPKLLIYGYYNRP |
| | | RVTMTRNTSISTAYMELSSLRSED | | SGVPDRFSGSKSGTSASLTI |
| | | TAVYYCAREVPETAAFEYWGQG | | TGLLPEDEGDYYCQSYDG |
| | | TLVTVSS | | TLSALFGGGTKLTVL |
| Antibody | 337 | QVQLVQSGAEVKKPGASVKVSC | 355 | EIVLTQSPGTLSASPGERVT |
| 13 | | KASGFDICDTYMHWVKQRPGQG | | LSCRASSSVSYMYWYQQK |
| | | LEWIGRIDPASGHTKYDPKFQVR | | PGQAPRPWIYATSNLASGV |
| | | ATITRDTSTSTAYLELSSLRSEDTA | | PDRFSGSGSGTDFTLTISRL |
| | | VYYCSRSGGLPDVWGQGTTVTV | | EPEDFAVYYCQQWSGNPR |
| | | SS | | TFGGGTKLEIK |
| Antibody | 338 | QVQLVQSGAEVVKPGASVKLSCK | 356 | EIVLTQSPGTLSLSPGERVT |
| 14 | | ASGFDICDTYMHWVRQRPGQGL | | MSCRASSSVSYMYWYQQ |
| | | EWMGRIDPASGHTKYDPKFQVR | | KPGQAPRPWIYATSNLASG |
| | | VTMTTDTSTSTVYLELSSLRSEDT | | VPDRFSGSGSGTDFTLTISR |
| | | AVYYCARSGGLPDVWGQGTTVT | | VEPEDFAVYYCQQWSGNP |
| | | VSS | | RTFGGGTKVEIK |
| Antibody | 339 | QVQLVQSGAEVKKPGASVKLSCK | 357 | EIVLTQSPGTLSLSPGERAT |
| 15 | | ASGFDICDTYMHWVRQRPGQGL | | MSCRASSSVSYMYWYQQ |
| | | EWIGRIDPASGHTKYDPKFQVRA | | KPGQAPRPWIYATSNLASG |
| | | TMTTDTSTSTVYLELSSLRSEDTA | | IPDRFSGSGSGTDFTLTISR |
| | | VYYCARSGGLPDVWGQGTTVTV | | VEPEDFAVYYCQQWSGNP |
| | | SS | | RTFGGGTKLEIK |
| Antibody | 340 | QVQLVQSGAEVKKPGASVKVSC | 358 | EIVLTQSPGTLSASPGERAT |
| 16 | | KASGFDICDTYMHWVKQRPGQG | | MSCRASSSVSYMYWYQQ |
| | | LEWIGRIDPASGHTKYDPKFQVR | | KPGQAPRPWIYATSNLASG |
| | | ATITTDTSTSTAYLELSSLRSEDTA | | VPDRFSGSGSGTDYTLTISR |
| | | VYYCARSGGLPDVWGQGTTVTV | | VEPEDFAVYYCQQWSGNP |
| | | SS | | RTFGGGTKLEIK |
| Antibody | 341 | QIQLVQSGPELKKPGETVKISCKA | 359 | DVLMTQTPLSLPVSLGDQ |
| 17 | | SGYTFTTYGMSWVKQAPGKGLK | | ASISCRSSQNIVHSDGNTYL |
| | | WMGWMNTYSGVTTYADDFKGR | | EWYLQKPGQSPKWYKVS |
| | | FAFSLETSASTAYMQIDNLKNEDT | | NRFSGVPDRFSGSGSGTDF |
| | | ATYFCAREGYVFDDYYATDYWG | | TLKISRVEAEDLGIYYCFQ |
| | | QGTSVTVSS | | GSHVPLTFGAGTKLELK |
| Antibody | 342 | QLQLQESGPGLVKPSETLSLTCTV | 360 | AIQLTQSPSSLSASVGDRV |
| 18 | | SGGSISSRSYYWGWIRQPPGKGLE | | TITCRASQGISSALAWYQQ |
| | | WIGSIYYNGRTYYNPSLKSRVTIS | | KPGKAPKLLIYDASSLESG |
| | | VDTSKNQFSLKLSSVTAADTAVY | | VPSRFSGSGSGTDFTLTISS |
| | | YCAREDYGDYGAFDIWGQGTMV | | LQPEDFATYYCQQFNSYPL |
| | | TVSS | | TFGGGTKVEIK |

In some embodiments, the TL1A binding protein comprises a heavy chain variable region (VH) comprising an amino acid sequence having at least 80% sequence identity with an amino acid sequence according to any one of SEQ ID NOs: 325-342. In some embodiments, the TL1A binding protein comprises a heavy chain variable region (VH) comprising an amino acid sequence having at least 85% sequence identity with an amino acid sequence according to any one of SEQ ID NOs: 325-342. In some embodiments, the TL1A binding protein comprises a heavy chain variable region (VH) comprising an amino acid sequence having at least 90% sequence identity with an amino acid sequence according to any one of SEQ ID Nos: 325-342. In some embodiments, the TL1A binding protein comprises a heavy chain variable region (VH) comprising an amino acid sequence having at least 95% sequence identity with an amino acid sequence according to any one of SEQ ID NOs: 325-342. In some embodiments, the TL1A binding protein comprises a heavy chain variable region (VH) comprising an amino acid sequence having at least 96% sequence identity with an amino acid sequence according to any one of SEQ ID NOs: 325-342. In some embodiments, the TL1A binding protein comprises a heavy chain variable region (VH) comprising an amino acid sequence having at least 97% sequence identity with an amino acid sequence according to any one of SEQ ID NOs: 325-342. In some embodiments, the TL1A binding protein comprises a heavy chain variable region (VH) comprising an amino acid sequence having at least 98% sequence identity with an amino acid sequence according to any one of SEQ ID NOs: 325-342. In some embodiments, the TL1A binding protein comprises a heavy chain variable region (VH) comprising an amino acid sequence having at least 99% sequence identity with an amino acid sequence according to any one of SEQ ID NOs: 325-342. In some embodiments, the TL1A binding protein comprises a heavy chain variable region (VH) comprising an amino acid sequence according to any one of SEQ ID NOs: 325-342.

In some embodiments, the TL1A binding protein comprises a light chain variable region (VL) comprising an amino acid sequence having at least 80% sequence identity with an amino acid sequence according to any one of SEQ ID NOs: 343-360. In some embodiments, the TL1A binding protein comprises a light chain variable region (VL) comprising an amino acid sequence having at least 85% sequence identity with an amino acid sequence according to any one of SEQ ID NOs: 343-360. In some embodiments, the TL1A binding protein antibody comprises a light chain variable region (VL) comprising an amino acid sequence having at least 90% sequence identity with an amino acid sequence according to any one of SEQ ID NOs: 343-360. In some embodiments, the TL1A binding protein comprises a light chain variable region (VL) comprising an amino acid sequence having at least 95% sequence identity with an amino acid sequence according to any one of SEQ ID NOs: 343-360. In some embodiments, the TL1A binding protein comprises a light chain variable region (VL) comprising an amino acid sequence having at least 96% sequence identity with an amino acid sequence according to any one of SEQ ID NOs: 343-360. In some embodiments, the TL1A binding protein comprises a light chain variable region (VL) comprising an amino acid sequence having at least 97% sequence identity with an amino acid sequence according to any one of SEQ ID NOs: 343-360. In some embodiments, the TL1A binding protein comprises a light chain variable region (VL) comprising an amino acid sequence having at least 98% sequence identity with an amino acid sequence according to any one of SEQ ID NOs: 343-360. In some embodiments, the TL1A binding protein comprises a light chain variable region (VL) comprising an amino acid sequence having at least 99% sequence identity with an amino acid sequence according to any one of SEQ ID NOs: 343-360. In some embodiments, the TL1A binding protein comprises a light chain variable region (VL) comprising an amino acid sequence according to any one of SEQ ID NOs: 343-360.

In some embodiments, the TL1A binding protein comprises a heavy chain variable region (VH) that comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to the heavy chain variable region (VH) of an TL1A binding protein disclosed in Table 2, and a light chain variable region (VL) that comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to the light chain variable region (VL) of the same TL1A binding protein disclosed in Table 2.

In some embodiments, the TL1A binding protein comprises a heavy chain variable region comprising an amino acid sequence having at least 80% sequence identity with an amino acid sequence according to any one of SEQ ID NOs: 325-342; and a light chain variable region comprising an amino acid sequence having at least 80% sequence identity with an amino acid sequence according to any one of SEQ ID NOs: 343-360. In some embodiments, the TL1A binding protein comprises a heavy chain variable region comprising an amino acid sequence having at least 85% sequence identity with an amino acid sequence according to any one of SEQ ID NOs: 325-342; and a light chain variable region comprising an amino acid sequence having at least 85% sequence identity with an amino acid sequence according to any one of SEQ ID NOs: 343-360. In some embodiments, the TL1A binding protein comprises a heavy chain variable region comprising an amino acid sequence having at least 90% sequence identity with an amino acid sequence according to any one of SEQ ID NOs: 325-342; and a light chain variable region comprising an amino acid sequence having at least 90% sequence identity with an amino acid sequence according to any one of SEQ ID NOs: 343-360. In some embodiments, the TL1A binding protein comprises a heavy chain variable region comprising an amino acid sequence having at least 95% sequence identity with an amino acid sequence according to any one of SEQ ID NOs: 325-342; and a light chain variable region comprising an amino acid sequence having at least 95% sequence identity with an amino acid sequence according to any one of SEQ ID NOs: 343-360. In some embodiments, the TL1A binding protein comprises a heavy chain variable region comprising an amino acid sequence having at least 96% sequence identity with an amino acid sequence according to any one of SEQ ID NOs: 325-342; and a light chain variable region comprising an amino acid sequence having at least 96% sequence identity with an amino acid sequence according to any one of SEQ ID NOs: 343-360. In some embodiments, the TL1A binding protein comprises a heavy chain variable region comprising an amino acid sequence having at least 97% sequence identity with an amino acid sequence according to any one of SEQ ID NOs: 325-342; and a light chain variable region comprising an amino acid sequence having at least 97% sequence identity with an amino acid sequence according to any one of SEQ ID NOs: 343-360. In some embodiments, the TL1A binding protein comprises a heavy chain variable region comprising an amino acid sequence having at least 98% sequence identity with an amino acid sequence according to any one of SEQ ID NOs: 325-342; and a light chain variable region comprising an amino acid sequence having at least 98% sequence identity with an amino acid sequence according to any one of SEQ ID NOs: 343-360. In some embodiments, the TL1A binding protein comprises a heavy chain variable region comprising an amino acid sequence having at least 99% sequence identity with an amino acid sequence according to any one of SEQ ID NOs: 325-342; and a light chain variable region comprising an amino acid sequence having at least 99% sequence identity with an amino acid sequence according any one of SEQ ID NOs: 343-360. In some embodiments, the TL1A binding protein comprises a heavy chain variable region comprising an amino acid sequence according to any one of SEQ ID NOs: 325-342; and a light chain variable region comprising an amino acid sequence according to any one of SEQ ID NOs: 343-360.

In some embodiments, the TL1A binding protein comprises a heavy chain variable region comprising an amino acid sequence having at least 80% sequence identity with an amino acid sequence according to SEQ ID NO: 325; and a light chain variable region comprising an amino acid sequence having at least 80% sequence identity with an amino acid sequence according to SEQ ID NO: 343. In some embodiments, the TL1A binding protein comprises a heavy chain variable region comprising an amino acid sequence having at least 85% sequence identity with an amino acid sequence according to SEQ ID NO: 325; and a light chain variable region comprising an amino acid sequence having at least 85% sequence identity with an amino acid sequence according to SEQ ID NO: 343. In some embodiments, the TL1A binding protein comprises a heavy chain variable region comprising an amino acid sequence having at least 90% sequence identity with an amino acid sequence according to SEQ ID NO: 325; and a light chain variable region comprising an amino acid sequence having at least 90% sequence identity with an amino acid sequence according to SEQ ID NO: 343. In some embodiments, the TL1A binding protein comprises a heavy chain variable region comprising an amino acid sequence having at least 95% sequence identity with an amino acid sequence according to SEQ ID NO: 325; and a light chain variable region comprising an amino acid sequence having at least 95% sequence identity with an amino acid sequence according to SEQ ID NO: 343. In some embodiments, the TL1A binding protein comprises a heavy chain variable region comprising an amino acid sequence having at least 96% sequence identity with an amino acid sequence according to SEQ ID NO: 325; and a light chain variable region comprising an amino acid sequence having at least 96% sequence identity with an amino acid sequence according to SEQ ID NO: 343. In some embodiments, the TL1A binding protein comprises a heavy chain variable region comprising an amino acid sequence having at least 97% sequence identity with an amino acid sequence according to SEQ ID NO: 325; and a light chain variable region comprising an amino acid sequence having at least 97% sequence identity with an amino acid sequence according to SEQ ID NO: 343. In some embodiments, the TL1A binding protein comprises a heavy chain variable region comprising an amino acid sequence having at least 98% sequence identity with an amino acid sequence according to SEQ ID NO: 325; and a light chain variable region comprising an amino acid sequence having at least 98% sequence identity with an amino acid sequence according to SEQ ID NO: 343. In some embodiments, the TL1A binding protein comprises a heavy chain variable region comprising an amino acid sequence having at least 99% sequence identity with an amino acid sequence according to SEQ ID NO: 325; and a light chain variable region comprising an amino acid sequence having at least 99% sequence identity with an amino acid sequence according SEQ ID NO: 343. In some embodiments, the TL1A binding protein comprises a heavy chain variable region comprising an amino acid sequence according to SEQ ID NO: 325; and a light chain variable region comprising an amino acid sequence according to SEQ ID NO: 343.

In some embodiments, the TL1A binding protein comprises a heavy chain variable region comprising an amino acid sequence having at least 80% sequence identity with an amino acid sequence according to SEQ ID NO: 326; and a light chain variable region comprising an amino acid sequence having at least 80% sequence identity with an amino acid sequence according to SEQ ID NO: 344. In some embodiments, the TL1A binding protein comprises a heavy chain variable region comprising an amino acid sequence having at least 85% sequence identity with an amino acid sequence according to SEQ ID NO: 326; and a light chain variable region comprising an amino acid sequence having at least 85% sequence identity with an amino acid sequence according to SEQ ID NO: 344. In some embodiments, the TL1A binding protein comprises a heavy chain variable region comprising an amino acid sequence having at least 90% sequence identity with an amino acid sequence according to SEQ ID NO: 326; and a light chain variable region comprising an amino acid sequence having at least 90% sequence identity with an amino acid sequence according to SEQ ID NO: 344. In some embodiments, the TL1A binding protein comprises a heavy chain variable region comprising an amino acid sequence having at least 95% sequence identity with an amino acid sequence according to SEQ ID NO: 326; and a light chain variable region comprising an amino acid sequence having at least 95% sequence identity with an amino acid sequence according to SEQ ID NO: 344. In some embodiments, the TL1A binding protein comprises a heavy chain variable region comprising an amino acid sequence having at least 96% sequence identity with an amino acid sequence according to SEQ ID NO: 326; and a light chain variable region comprising an amino acid sequence having at least 96% sequence identity with an amino acid sequence according to SEQ ID NO: 344. In some embodiments, the TL1A binding protein comprises a heavy chain variable region comprising an amino acid sequence having at least 97% sequence identity with an amino acid sequence according to SEQ ID NO: 326; and a light chain variable region comprising an amino acid sequence having at least 97% sequence identity with an amino acid sequence according to SEQ ID NO: 344. In some embodiments, the TL1A binding protein comprises a heavy chain variable region comprising an amino acid sequence having at least 98% sequence identity with an amino acid sequence according to SEQ ID NO: 326; and a light chain variable region comprising an amino acid sequence having at least 98% sequence identity with an amino acid sequence according to SEQ ID NO: 344. In some embodiments, the TL1A binding protein comprises a heavy chain variable region comprising an amino acid sequence having at least 99% sequence identity with an amino acid sequence according to SEQ ID NO: 326; and a light chain variable region comprising an amino acid sequence having at least 99% sequence identity with an amino acid sequence according SEQ ID NO: 344. In some embodiments, the TL1A binding protein comprises a heavy chain variable region comprising an amino acid sequence according to SEQ ID NO: 326; and a light chain variable region comprising an amino acid sequence according to SEQ ID NO: 344.

In some embodiments, the TL1A binding protein comprises a heavy chain variable region comprising an amino acid sequence having at least 80% sequence identity with an amino acid sequence according to SEQ ID NO: 327; and a light chain variable region comprising an amino acid sequence having at least 80% sequence identity with an amino acid sequence according to SEQ ID NO: 345. In some embodiments, the TL1A binding protein comprises a heavy chain variable region comprising an amino acid sequence having at least 85% sequence identity with an amino acid sequence according to SEQ ID NO: 327; and a light chain variable region comprising an amino acid sequence having at least 85% sequence identity with an amino acid sequence according to SEQ ID NO: 345. In some embodiments, the TL1A binding protein comprises a heavy chain variable region comprising an amino acid sequence having at least 90% sequence identity with an amino acid sequence according to SEQ ID NO: 327; and a light chain variable region comprising an amino acid sequence having at least 90% sequence identity with an amino acid sequence according to SEQ ID NO: 345. In some embodiments, the TL1A binding protein comprises a heavy chain variable region comprising an amino acid sequence having at least 95% sequence identity with an amino acid sequence according to SEQ ID NO: 327; and a light chain variable region comprising an amino acid sequence having at least 95% sequence identity with an amino acid sequence according to SEQ ID NO: 345. In some embodiments, the TL1A binding protein comprises a heavy chain variable region comprising an amino acid sequence having at least 96% sequence identity with an amino acid sequence according to SEQ ID NO: 327; and a light chain variable region comprising an amino acid sequence having at least 96% sequence identity with an amino acid sequence according to SEQ ID NO: 345. In some embodiments, the TL1A binding protein comprises a heavy chain variable region comprising an amino acid sequence having at least 97% sequence identity with an amino acid sequence according to SEQ ID NO: 327; and a light chain variable region comprising an amino acid sequence having at least 97% sequence identity with an amino acid sequence according to SEQ ID NO: 345. In some embodiments, the TL1A binding protein comprises a heavy chain variable region comprising an amino acid sequence having at least 98% sequence identity with an amino acid sequence according to SEQ ID NO: 327; and a light chain variable region comprising an amino acid sequence having at least 98% sequence identity with an amino acid sequence according to SEQ ID NO: 345. In some embodiments, the TL1A binding protein comprises a heavy chain variable region comprising an amino acid sequence having at least 99% sequence identity with an amino acid sequence according to SEQ ID NO: 327; and a light chain variable region comprising an amino acid sequence having at least 99% sequence identity with an amino acid sequence according SEQ ID NO: 345. In some embodiments, the TL1A binding protein comprises a heavy chain variable region comprising an amino acid sequence according to SEQ ID NO: 327; and a light chain variable region comprising an amino acid sequence according to SEQ ID NO: 345.

In some embodiments, the TL1A binding protein comprises a heavy chain variable region comprising an amino acid sequence having at least 80% sequence identity with an amino acid sequence according to SEQ ID NO: 328; and a light chain variable region comprising an amino acid sequence having at least 80% sequence identity with an amino acid sequence according to SEQ ID NO: 346. In some embodiments, the TL1A binding protein comprises a heavy chain variable region comprising an amino acid sequence having at least 85% sequence identity with an amino acid sequence according to SEQ ID NO: 328; and a light chain variable region comprising an amino acid sequence having at least 85% sequence identity with an amino acid sequence according to SEQ ID NO: 346. In some embodiments, the TL1A binding protein comprises a heavy chain variable region comprising an amino acid sequence having at least 90% sequence identity with an amino acid sequence according to SEQ ID NO: 328; and a light chain variable region comprising an amino acid sequence having at least 90% sequence identity with an amino acid sequence according to SEQ ID NO: 346. In some embodiments, the TL1A binding protein comprises a heavy chain variable region comprising an amino acid sequence having at least 95% sequence identity with an amino acid sequence according to SEQ ID NO: 328; and a light chain variable region comprising an amino acid sequence having at least 95% sequence identity with an amino acid sequence according to SEQ ID NO: 346. In some embodiments, the TL1A binding protein comprises a heavy chain variable region comprising an amino acid sequence having at least 96% sequence identity with an amino acid sequence according to SEQ ID NO: 328; and a light chain variable region comprising an amino acid sequence having at least 96% sequence identity with an amino acid sequence according to SEQ ID NO: 346. In some embodiments, the TL1A binding protein comprises a heavy chain variable region comprising an amino acid sequence having at least 97% sequence identity with an amino acid sequence according to SEQ ID NO: 328; and a light chain variable region comprising an amino acid sequence having at least 97% sequence identity with an amino acid sequence according to SEQ ID NO: 346. In some embodiments, the TL1A binding protein comprises a heavy chain variable region comprising an amino acid sequence having at least 98% sequence identity with an amino acid sequence according to SEQ ID NO: 328; and a light chain variable region comprising an amino acid sequence having at least 98% sequence identity with an amino acid sequence according to SEQ ID NO: 346. In some embodiments, the TL1A binding protein comprises a heavy chain variable region comprising an amino acid sequence having at least 99% sequence identity with an amino acid sequence according to SEQ ID NO: 328; and a light chain variable region comprising an amino acid sequence having at least 99% sequence identity with an amino acid sequence according SEQ ID NO: 346. In some embodiments, the TL1A binding protein comprises a heavy chain variable region comprising an amino acid sequence according to SEQ ID NO: 328; and a light chain variable region comprising an amino acid sequence according to SEQ ID NO: 346.

In some embodiments, the TL1A binding protein comprises a heavy chain variable region comprising an amino acid sequence having at least 80% sequence identity with an amino acid sequence according to SEQ ID NO: 329; and a light chain variable region comprising an amino acid sequence having at least 80% sequence identity with an amino acid sequence according to SEQ ID NO: 347. In some embodiments, the TL1A binding protein comprises a heavy chain variable region comprising an amino acid sequence having at least 85% sequence identity with an amino acid sequence according to SEQ ID NO: 329; and a light chain variable region comprising an amino acid sequence having at least 85% sequence identity with an amino acid sequence according to SEQ ID NO: 347. In some embodiments, the TL1A binding protein comprises a heavy chain variable region comprising an amino acid sequence having at least 90% sequence identity with an amino acid sequence according to SEQ ID NO: 329; and a light chain variable region comprising an amino acid sequence having at least 90% sequence identity with an amino acid sequence according to SEQ ID NO: 347. In some embodiments, the TL1A binding protein comprises a heavy chain variable region comprising an amino acid sequence having at least 95% sequence identity with an amino acid sequence according to SEQ ID NO: 329; and a light chain variable region comprising an amino acid sequence having at least 95% sequence identity with an amino acid sequence according to SEQ ID NO: 347. In some embodiments, the TL1A binding protein comprises a heavy chain variable region comprising an amino acid sequence having at least 96% sequence identity with an amino acid sequence according to SEQ ID NO: 329; and a light chain variable region comprising an amino acid sequence having at least 96% sequence identity with an amino acid sequence according to SEQ ID NO: 347. In some embodiments, the TL1A binding protein comprises a heavy chain variable region comprising an amino acid sequence having at least 97% sequence identity with an amino acid sequence according to SEQ ID NO: 329; and a light chain variable region comprising an amino acid sequence having at least 97% sequence identity with an amino acid sequence according to SEQ ID NO: 347. In some embodiments, the TL1A binding protein comprises a heavy chain variable region comprising an amino acid sequence having at least 98% sequence identity with an amino acid sequence according to SEQ ID NO: 329; and a light chain variable region comprising an amino acid sequence having at least 98% sequence identity with an amino acid sequence according to SEQ ID NO: 347. In some embodiments, the TL1A binding protein comprises a heavy chain variable region comprising an amino acid sequence having at least 99% sequence identity with an amino acid sequence according to SEQ ID NO: 329; and a light chain variable region comprising an amino acid sequence having at least 99% sequence identity with an amino acid sequence according SEQ ID NO: 347. In some embodiments, the TL1A binding protein comprises a heavy chain variable region comprising an amino acid sequence according to SEQ ID NO: 329; and a light chain variable region comprising an amino acid sequence according to SEQ ID NO: 347.

In some embodiments, the TL1A binding protein comprises a heavy chain variable region comprising an amino acid sequence having at least 80% sequence identity with an amino acid sequence according to SEQ ID NO: 330; and a light chain variable region comprising an amino acid sequence having at least 80% sequence identity with an amino acid sequence according to SEQ ID NO: 348. In some embodiments, the TL1A binding protein comprises a heavy chain variable region comprising an amino acid sequence having at least 85% sequence identity with an amino acid sequence according to SEQ ID NO: 330; and a light chain variable region comprising an amino acid sequence having at least 85% sequence identity with an amino acid sequence according to SEQ ID NO: 348. In some embodiments, the TL1A binding protein comprises a heavy chain variable region comprising an amino acid sequence having at least 90% sequence identity with an amino acid sequence according to SEQ ID NO: 330; and a light chain variable region comprising an amino acid sequence having at least 90% sequence identity with an amino acid sequence according to SEQ ID NO: 348. In some embodiments, the TL1A binding protein comprises a heavy chain variable region comprising an amino acid sequence having at least 95% sequence identity with an amino acid sequence according to SEQ ID NO: 330; and a light chain variable region comprising an amino acid sequence having at least 95% sequence identity with an amino acid sequence according to SEQ ID NO: 348. In some embodiments, the TL1A binding protein comprises a heavy chain variable region comprising an amino acid sequence having at least 96% sequence identity with an amino acid sequence according to SEQ ID NO: 330; and a light chain variable region comprising an amino acid sequence having at least 96% sequence identity with an amino acid sequence according to SEQ ID NO: 348. In some embodiments, the TL1A binding protein comprises a heavy chain variable region comprising an amino acid sequence having at least 97% sequence identity with an amino acid sequence according to SEQ ID NO: 330; and a light chain variable region comprising an amino acid sequence having at least 97% sequence identity with an amino acid sequence according to SEQ ID NO: 348. In some embodiments, the TL1A binding protein comprises a heavy chain variable region comprising an amino acid sequence having at least 98% sequence identity with an amino acid sequence according to SEQ ID NO: 330; and a light chain variable region comprising an amino acid sequence having at least 98% sequence identity with an amino acid sequence according to SEQ ID NO: 348. In some embodiments, the TL1A binding protein comprises a heavy chain variable region comprising an amino acid sequence having at least 99% sequence identity with an amino acid sequence according to SEQ ID NO: 330; and a light chain variable region comprising an amino acid sequence having at least 99% sequence identity with an amino acid sequence according SEQ ID NO: 348. In some embodiments, the TL1A binding protein comprises a heavy chain variable region comprising an amino acid sequence according to SEQ ID NO: 330; and a light chain variable region comprising an amino acid sequence according to SEQ ID NO: 348.

In some embodiments, the TL1A binding protein comprises a heavy chain variable region comprising an amino acid sequence having at least 80% sequence identity with an amino acid sequence according to SEQ ID NO: 331; and a light chain variable region comprising an amino acid sequence having at least 80% sequence identity with an amino acid sequence according to SEQ ID NO: 349. In some embodiments, the TL1A binding protein comprises a heavy chain variable region comprising an amino acid sequence having at least 85% sequence identity with an amino acid sequence according to SEQ ID NO: 331; and a light chain variable region comprising an amino acid sequence having at least 85% sequence identity with an amino acid sequence according to SEQ ID NO: 349. In some embodiments, the TL1A binding protein comprises a heavy chain variable region comprising an amino acid sequence having at least 90% sequence identity with an amino acid sequence according to SEQ ID NO: 331; and a light chain variable region comprising an amino acid sequence having at least 90% sequence identity with an amino acid sequence according to SEQ ID NO: 349. In some embodiments, the TL1A binding protein comprises a heavy chain variable region comprising an amino acid sequence having at least 95% sequence identity with an amino acid sequence according to SEQ ID NO: 331; and a light chain variable region comprising an amino acid sequence having at least 95% sequence identity with an amino acid sequence according to SEQ ID NO: 349. In some embodiments, the TL1A binding protein comprises a heavy chain variable region comprising an amino acid sequence having at least 96% sequence identity with an amino acid sequence according to SEQ ID NO: 331; and a light chain variable region comprising an amino acid sequence having at least 96% sequence identity with an amino acid sequence according to SEQ ID NO: 349. In some embodiments, the TL1A binding protein comprises a heavy chain variable region comprising an amino acid sequence having at least 97% sequence identity with an amino acid sequence according to SEQ ID NO: 331; and a light chain variable region comprising an amino acid sequence having at least 97% sequence identity with an amino acid sequence according to SEQ ID NO: 349. In some embodiments, the TL1A binding protein comprises a heavy chain variable region comprising an amino acid sequence having at least 98% sequence identity with an amino acid sequence according to SEQ ID NO: 331; and a light chain variable region comprising an amino acid sequence having at least 98% sequence identity with an amino acid sequence according to SEQ ID NO: 349. In some embodiments, the TL1A binding protein comprises a heavy chain variable region comprising an amino acid sequence having at least 99% sequence identity with an amino acid sequence according to SEQ ID NO: 331; and a light chain variable region comprising an amino acid sequence having at least 99% sequence identity with an amino acid sequence according SEQ ID NO: 349. In some embodiments, the TL1A binding protein comprises a heavy chain variable region comprising an amino acid sequence according to SEQ ID NO: 331; and a light chain variable region comprising an amino acid sequence according to SEQ ID NO: 349.

In some embodiments, the TL1A binding protein comprises a heavy chain variable region comprising an amino acid sequence having at least 80% sequence identity with an amino acid sequence according to SEQ ID NO: 332; and a light chain variable region comprising an amino acid sequence having at least 80% sequence identity with an amino acid sequence according to SEQ ID NO: 350. In some embodiments, the TL1A binding protein comprises a heavy chain variable region comprising an amino acid sequence having at least 85% sequence identity with an amino acid sequence according to SEQ ID NO: 332; and a light chain variable region comprising an amino acid sequence having at least 85% sequence identity with an amino acid sequence according to SEQ ID NO: 350. In some embodiments, the TL1A binding protein comprises a heavy chain variable region comprising an amino acid sequence having at least 90% sequence identity with an amino acid sequence according to SEQ ID NO: 332; and a light chain variable region comprising an amino acid sequence having at least 90% sequence identity with an amino acid sequence according to SEQ ID NO: 350. In some embodiments, the TL1A binding protein comprises a heavy chain variable region comprising an amino acid sequence having at least 95% sequence identity with an amino acid sequence according to SEQ ID NO: 332; and a light chain variable region comprising an amino acid sequence having at least 95% sequence identity with an amino acid sequence according to SEQ ID NO: 350. In some embodiments, the TL1A binding protein comprises a heavy chain variable region comprising an amino acid sequence having at least 96% sequence identity with an amino acid sequence according to SEQ ID NO: 332; and a light chain variable region comprising an amino acid sequence having at least 96% sequence identity with an amino acid sequence according to SEQ ID NO: 350. In some embodiments, the TL1A binding protein comprises a heavy chain variable region comprising an amino acid sequence having at least 97% sequence identity with an amino acid sequence according to SEQ ID NO: 332; and a light chain variable region comprising an amino acid sequence having at least 97% sequence identity with an amino acid sequence according to SEQ ID NO: 350. In some embodiments, the TL1A binding protein comprises a heavy chain variable region comprising an amino acid sequence having at least 98% sequence identity with an amino acid sequence according to SEQ ID NO: 332; and a light chain variable region comprising an amino acid sequence having at least 98% sequence identity with an amino acid sequence according to SEQ ID NO: 350. In some embodiments, the TL1A binding protein comprises a heavy chain variable region comprising an amino acid sequence having at least 99% sequence identity with an amino acid sequence according to SEQ ID NO: 332; and a light chain variable region comprising an amino acid sequence having at least 99% sequence identity with an amino acid sequence according SEQ ID NO: 350. In some embodiments, the TL1A binding protein comprises a heavy chain variable region comprising an amino acid sequence according to SEQ ID NO: 332; and a light chain variable region comprising an amino acid sequence according to SEQ ID NO: 350.

In some embodiments, the TL1A binding protein comprises a heavy chain variable region comprising an amino acid sequence having at least 80% sequence identity with an amino acid sequence according to SEQ ID NO: 333; and a light chain variable region comprising an amino acid sequence having at least 80% sequence identity with an amino acid sequence according to SEQ ID NO: 351. In some embodiments, the TL1A binding protein comprises a heavy chain variable region comprising an amino acid sequence having at least 85% sequence identity with an amino acid sequence according to SEQ ID NO: 333; and a light chain variable region comprising an amino acid sequence having at least 85% sequence identity with an amino acid sequence according to SEQ ID NO: 351. In some embodiments, the TL1A binding protein comprises a heavy chain variable region comprising an amino acid sequence having at least 90% sequence identity with an amino acid sequence according to SEQ ID NO: 333; and a light chain variable region comprising an amino acid sequence having at least 90% sequence identity with an amino acid sequence according to SEQ ID NO: 351. In some embodiments, the TL1A binding protein comprises a heavy chain variable region comprising an amino acid sequence having at least 95% sequence identity with an amino acid sequence according to SEQ ID NO: 333; and a light chain variable region comprising an amino acid sequence having at least 95% sequence identity with an amino acid sequence according to SEQ ID NO: 351. In some embodiments, the TL1A binding protein comprises a heavy chain variable region comprising an amino acid sequence having at least 96% sequence identity with an amino acid sequence according to SEQ ID NO: 333; and a light chain variable region comprising an amino acid sequence having at least 96% sequence identity with an amino acid sequence according to SEQ ID NO: 351. In some embodiments, the TL1A binding protein comprises a heavy chain variable region comprising an amino acid sequence having at least 97% sequence identity with an amino acid sequence according to SEQ ID NO: 333; and a light chain variable region comprising an amino acid sequence having at least 97% sequence identity with an amino acid sequence according to SEQ ID NO: 351. In some embodiments, the TL1A binding protein comprises a heavy chain variable region comprising an amino acid sequence having at least 98% sequence identity with an amino acid sequence according to SEQ ID NO: 333; and a light chain variable region comprising an amino acid sequence having at least 98% sequence identity with an amino acid sequence according to SEQ ID NO: 351. In some embodiments, the TL1A binding protein comprises a heavy chain variable region comprising an amino acid sequence having at least 99% sequence identity with an amino acid sequence according to SEQ ID NO: 333; and a light chain variable region comprising an amino acid sequence having at least 99% sequence identity with an amino acid sequence according SEQ ID NO: 351. In some embodiments, the TL1A binding protein comprises a heavy chain variable region comprising an amino acid sequence according to SEQ ID NO: 333; and a light chain variable region comprising an amino acid sequence according to SEQ ID NO: 351.

In some embodiments, the TL1A binding protein comprises a heavy chain variable region comprising an amino acid sequence having at least 80% sequence identity with an amino acid sequence according to SEQ ID NO: 334; and a light chain variable region comprising an amino acid sequence having at least 80% sequence identity with an amino acid sequence according to SEQ ID NO: 352. In some embodiments, the TL1A binding protein comprises a heavy chain variable region comprising an amino acid sequence having at least 85% sequence identity with an amino acid sequence according to SEQ ID NO: 334; and a light chain variable region comprising an amino acid sequence having at least 85% sequence identity with an amino acid sequence according to SEQ ID NO: 352. In some embodiments, the TL1A binding protein comprises a heavy chain variable region comprising an amino acid sequence having at least 90% sequence identity with an amino acid sequence according to SEQ ID NO: 334; and a light chain variable region comprising an amino acid sequence having at least 90% sequence identity with an amino acid sequence according to SEQ ID NO: 352. In some embodiments, the TL1A binding protein comprises a heavy chain variable region comprising an amino acid sequence having at least 95% sequence identity with an amino acid sequence according to SEQ ID NO: 334; and a light chain variable region comprising an amino acid sequence having at least 95% sequence identity with an amino acid sequence according to SEQ ID NO: 352. In some embodiments, the TL1A binding protein comprises a heavy chain variable region comprising an amino acid sequence having at least 96% sequence identity with an amino acid sequence according to SEQ ID NO: 334; and a light chain variable region comprising an amino acid sequence having at least 96% sequence identity with an amino acid sequence according to SEQ ID NO: 352. In some embodiments, the TL1A binding protein comprises a heavy chain variable region comprising an amino acid sequence having at least 97% sequence identity with an amino acid sequence according to SEQ ID NO: 334; and a light chain variable region comprising an amino acid sequence having at least 97% sequence identity with an amino acid sequence according to SEQ ID NO: 352. In some embodiments, the TL1A binding protein comprises a heavy chain variable region comprising an amino acid sequence having at least 98% sequence identity with an amino acid sequence according to SEQ ID NO: 334; and a light chain variable region comprising an amino acid sequence having at least 98% sequence identity with an amino acid sequence according to SEQ ID NO: 352. In some embodiments, the TL1A binding protein comprises a heavy chain variable region comprising an amino acid sequence having at least 99% sequence identity with an amino acid sequence according to SEQ ID NO: 334; and a light chain variable region comprising an amino acid sequence having at least 99% sequence identity with an amino acid sequence according SEQ ID NO: 352. In some embodiments, the TL1A binding protein comprises a heavy chain variable region comprising an amino acid sequence according to SEQ ID NO: 334; and a light chain variable region comprising an amino acid sequence according to SEQ ID NO: 352.

In some embodiments, the TL1A binding protein comprises a heavy chain variable region comprising an amino acid sequence having at least 80% sequence identity with an amino acid sequence according to SEQ ID NO: 335; and a light chain variable region comprising an amino acid sequence having at least 80% sequence identity with an amino acid sequence according to SEQ ID NO: 353. In some embodiments, the TL1A binding protein comprises a heavy chain variable region comprising an amino acid sequence having at least 85% sequence identity with an amino acid sequence according to SEQ ID NO: 335; and a light chain variable region comprising an amino acid sequence having at least 85% sequence identity with an amino acid sequence according to SEQ ID NO: 353. In some embodiments, the TL1A binding protein comprises a heavy chain variable region comprising an amino acid sequence having at least 90% sequence identity with an amino acid sequence according to SEQ ID NO: 335; and a light chain variable region comprising an amino acid sequence having at least 90% sequence identity with an amino acid sequence according to SEQ ID NO: 353. In some embodiments, the TL1A binding protein comprises a heavy chain variable region comprising an amino acid sequence having at least 95% sequence identity with an amino acid sequence according to SEQ ID NO: 335; and a light chain variable region comprising an amino acid sequence having at least 95% sequence identity with an amino acid sequence according to SEQ ID NO: 353. In some embodiments, the TL1A binding protein comprises a heavy chain variable region comprising an amino acid sequence having at least 96% sequence identity with an amino acid sequence according to SEQ ID NO: 335; and a light chain variable region comprising an amino acid sequence having at least 96% sequence identity with an amino acid sequence according to SEQ ID NO: 353. In some embodiments, the TL1A binding protein comprises a heavy chain variable region comprising an amino acid sequence having at least 97% sequence identity with an amino acid sequence according to SEQ ID NO: 335; and a light chain variable region comprising an amino acid sequence having at least 97% sequence identity with an amino acid sequence according to SEQ ID NO: 353. In some embodiments, the TL1A binding protein comprises a heavy chain variable region comprising an amino acid sequence having at least 98% sequence identity with an amino acid sequence according to SEQ ID NO: 335; and a light chain variable region comprising an amino acid sequence having at least 98% sequence identity with an amino acid sequence according to SEQ ID NO: 353. In some embodiments, the TL1A binding protein comprises a heavy chain variable region comprising an amino acid sequence having at least 99% sequence identity with an amino acid sequence according to SEQ ID NO: 335; and a light chain variable region comprising an amino acid sequence having at least 99% sequence identity with an amino acid sequence according SEQ ID NO: 353. In some embodiments, the TL1A binding protein comprises a heavy chain variable region comprising an amino acid sequence according to SEQ ID NO: 335; and a light chain variable region comprising an amino acid sequence according to SEQ ID NO: 353.

In some embodiments, the TL1A binding protein comprises a heavy chain variable region comprising an amino acid sequence having at least 80% sequence identity with an amino acid sequence according to SEQ ID NO: 336; and a light chain variable region comprising an amino acid sequence having at least 80% sequence identity with an amino acid sequence according to SEQ ID NO: 354. In some embodiments, the TL1A binding protein comprises a heavy chain variable region comprising an amino acid sequence having at least 85% sequence identity with an amino acid sequence according to SEQ ID NO: 336; and a light chain variable region comprising an amino acid sequence having at least 85% sequence identity with an amino acid sequence according to SEQ ID NO: 354. In some embodiments, the TL1A binding protein comprises a heavy chain variable region comprising an amino acid sequence having at least 90% sequence identity with an amino acid sequence according to SEQ ID NO: 336; and a light chain variable region comprising an amino acid sequence having at least 90% sequence identity with an amino acid sequence according to SEQ ID NO: 354. In some embodiments, the TL1A binding protein comprises a heavy chain variable region comprising an amino acid sequence having at least 95% sequence identity with an amino acid sequence according to SEQ ID NO: 336; and a light chain variable region comprising an amino acid sequence having at least 95% sequence identity with an amino acid sequence according to SEQ ID NO: 354. In some embodiments, the TL1A binding protein comprises a heavy chain variable region comprising an amino acid sequence having at least 96% sequence identity with an amino acid sequence according to SEQ ID NO: 336; and a light chain variable region comprising an amino acid sequence having at least 96% sequence identity with an amino acid sequence according to SEQ ID NO: 354. In some embodiments, the TL1A binding protein comprises a heavy chain variable region comprising an amino acid sequence having at least 97% sequence identity with an amino acid sequence according to SEQ ID NO: 336; and a light chain variable region comprising an amino acid sequence having at least 97% sequence identity with an amino acid sequence according to SEQ ID NO: 354. In some embodiments, the TL1A binding protein comprises a heavy chain variable region comprising an amino acid sequence having at least 98% sequence identity with an amino acid sequence according to SEQ ID NO: 336; and a light chain variable region comprising an amino acid sequence having at least 98% sequence identity with an amino acid sequence according to SEQ ID NO: 354. In some embodiments, the TL1A binding protein comprises a heavy chain variable region comprising an amino acid sequence having at least 99% sequence identity with an amino acid sequence according to SEQ ID NO: 336; and a light chain variable region comprising an amino acid sequence having at least 99% sequence identity with an amino acid sequence according SEQ ID NO: 354. In some embodiments, the TL1A binding protein comprises a heavy chain variable region comprising an amino acid sequence according to SEQ ID NO: 336; and a light chain variable region comprising an amino acid sequence according to SEQ ID NO: 354.

In some embodiments, the TL1A binding protein comprises a heavy chain variable region comprising an amino acid sequence having at least 80% sequence identity with an amino acid sequence according to SEQ ID NO: 337; and a light chain variable region comprising an amino acid sequence having at least 80% sequence identity with an amino acid sequence according to SEQ ID NO: 355. In some embodiments, the TL1A binding protein comprises a heavy chain variable region comprising an amino acid sequence having at least 85% sequence identity with an amino acid sequence according to SEQ ID NO: 337; and a light chain variable region comprising an amino acid sequence having at least 85% sequence identity with an amino acid sequence according to SEQ ID NO: 355. In some embodiments, the TL1A binding protein comprises a heavy chain variable region comprising an amino acid sequence having at least 90% sequence identity with an amino acid sequence according to SEQ ID NO: 337; and a light chain variable region comprising an amino acid sequence having at least 90% sequence identity with an amino acid sequence according to SEQ ID NO: 355. In some embodiments, the TL1A binding protein comprises a heavy chain variable region comprising an amino acid sequence having at least 95% sequence identity with an amino acid sequence according to SEQ ID NO: 337; and a light chain variable region comprising an amino acid sequence having at least 95% sequence identity with an amino acid sequence according to SEQ ID NO: 355. In some embodiments, the TL1A binding protein comprises a heavy chain variable region comprising an amino acid sequence having at least 96% sequence identity with an amino acid sequence according to SEQ ID NO: 337; and a light chain variable region comprising an amino acid sequence having at least 96% sequence identity with an amino acid sequence according to SEQ ID NO: 355. In some embodiments, the TL1A binding protein comprises a heavy chain variable region comprising an amino acid sequence having at least 97% sequence identity with an amino acid sequence according to SEQ ID NO: 337; and a light chain variable region comprising an amino acid sequence having at least 97% sequence identity with an amino acid sequence according to SEQ ID NO: 355. In some embodiments, the TL1A binding protein comprises a heavy chain variable region comprising an amino acid sequence having at least 98% sequence identity with an amino acid sequence according to SEQ ID NO: 337; and a light chain variable region comprising an amino acid sequence having at least 98% sequence identity with an amino acid sequence according to SEQ ID NO: 355. In some embodiments, the TL1A binding protein comprises a heavy chain variable region comprising an amino acid sequence having at least 99% sequence identity with an amino acid sequence according to SEQ ID NO: 337; and a light chain variable region comprising an amino acid sequence having at least 99% sequence identity with an amino acid sequence according SEQ ID NO: 355. In some embodiments, the TL1A binding protein comprises a heavy chain variable region comprising an amino acid sequence according to SEQ ID NO: 337; and a light chain variable region comprising an amino acid sequence according to SEQ ID NO: 355.

In some embodiments, the TL1A binding protein comprises a heavy chain variable region comprising an amino acid sequence having at least 80% sequence identity with an amino acid sequence according to SEQ ID NO: 338; and a light chain variable region comprising an amino acid sequence having at least 80% sequence identity with an amino acid sequence according to SEQ ID NO: 356. In some embodiments, the TL1A binding protein comprises a heavy chain variable region comprising an amino acid sequence having at least 85% sequence identity with an amino acid sequence according to SEQ ID NO: 338; and a light chain variable region comprising an amino acid sequence having at least 85% sequence identity with an amino acid sequence according to SEQ ID NO: 356. In some embodiments, the TL1A binding protein comprises a heavy chain variable region comprising an amino acid sequence having at least 90% sequence identity with an amino acid sequence according to SEQ ID NO: 338; and a light chain variable region comprising an amino acid sequence having at least 90% sequence identity with an amino acid sequence according to SEQ ID NO: 356. In some embodiments, the TL1A binding protein comprises a heavy chain variable region comprising an amino acid sequence having at least 95% sequence identity with an amino acid sequence according to SEQ ID NO: 338; and a light chain variable region comprising an amino acid sequence having at least 95% sequence identity with an amino acid sequence according to SEQ ID NO: 356. In some embodiments, the TL1A binding protein comprises a heavy chain variable region comprising an amino acid sequence having at least 96% sequence identity with an amino acid sequence according to SEQ ID NO: 338; and a light chain variable region comprising an amino acid sequence having at least 96% sequence identity with an amino acid sequence according to SEQ ID NO: 356. In some embodiments, the TL1A binding protein comprises a heavy chain variable region comprising an amino acid sequence having at least 97% sequence identity with an amino acid sequence according to SEQ ID NO: 338; and a light chain variable region comprising an amino acid sequence having at least 97% sequence identity with an amino acid sequence according to SEQ ID NO: 356. In some embodiments, the TL1A binding protein comprises a heavy chain variable region comprising an amino acid sequence having at least 98% sequence identity with an amino acid sequence according to SEQ ID NO: 338; and a light chain variable region comprising an amino acid sequence having at least 98% sequence identity with an amino acid sequence according to SEQ ID NO: 356. In some embodiments, the TL1A binding protein comprises a heavy chain variable region comprising an amino acid sequence having at least 99% sequence identity with an amino acid sequence according to SEQ ID NO: 338; and a light chain variable region comprising an amino acid sequence having at least 99% sequence identity with an amino acid sequence according SEQ ID NO: 356. In some embodiments, the TL1A binding protein comprises a heavy chain variable region comprising an amino acid sequence according to SEQ ID NO: 338; and a light chain variable region comprising an amino acid sequence according to SEQ ID NO: 356.

In some embodiments, the TL1A binding protein comprises a heavy chain variable region comprising an amino acid sequence having at least 80% sequence identity with an amino acid sequence according to SEQ ID NO: 339; and a light chain variable region comprising an amino acid sequence having at least 80% sequence identity with an amino acid sequence according to SEQ ID NO: 357. In some embodiments, the TL1A binding protein comprises a heavy chain variable region comprising an amino acid sequence having at least 85% sequence identity with an amino acid sequence according to SEQ ID NO: 339; and a light chain variable region comprising an amino acid sequence having at least 85% sequence identity with an amino acid sequence according to SEQ ID NO: 357. In some embodiments, the TL1A binding protein comprises a heavy chain variable region comprising an amino acid sequence having at least 90% sequence identity with an amino acid sequence according to SEQ ID NO: 339; and a light chain variable region comprising an amino acid sequence having at least 90% sequence identity with an amino acid sequence according to SEQ ID NO: 357. In some embodiments, the TL1A binding protein comprises a heavy chain variable region comprising an amino acid sequence having at least 95% sequence identity with an amino acid sequence according to SEQ ID NO: 339; and a light chain variable region comprising an amino acid sequence having at least 95% sequence identity with an amino acid sequence according to SEQ ID NO: 357. In some embodiments, the TL1A binding protein comprises a heavy chain variable region comprising an amino acid sequence having at least 96% sequence identity with an amino acid sequence according to SEQ ID NO: 339; and a light chain variable region comprising an amino acid sequence having at least 96% sequence identity with an amino acid sequence according to SEQ ID NO: 357. In some embodiments, the TL1A binding protein comprises a heavy chain variable region comprising an amino acid sequence having at least 97% sequence identity with an amino acid sequence according to SEQ ID NO: 339; and a light chain variable region comprising an amino acid sequence having at least 97% sequence identity with an amino acid sequence according to SEQ ID NO: 357. In some embodiments, the TL1A binding protein comprises a heavy chain variable region comprising an amino acid sequence having at least 98% sequence identity with an amino acid sequence according to SEQ ID NO: 339; and a light chain variable region comprising an amino acid sequence having at least 98% sequence identity with an amino acid sequence according to SEQ ID NO: 357. In some embodiments, the TL1A binding protein comprises a heavy chain variable region comprising an amino acid sequence having at least 99% sequence identity with an amino acid sequence according to SEQ ID NO: 339; and a light chain variable region comprising an amino acid sequence having at least 99% sequence identity with an amino acid sequence according SEQ ID NO: 357. In some embodiments, the TL1A binding protein comprises a heavy chain variable region comprising an amino acid sequence according to SEQ ID NO: 339; and a light chain variable region comprising an amino acid sequence according to SEQ ID NO: 357.

In some embodiments, the TL1A binding protein comprises a heavy chain variable region comprising an amino acid sequence having at least 80% sequence identity with an amino acid sequence according to SEQ ID NO: 340; and a light chain variable region comprising an amino acid sequence having at least 80% sequence identity with an amino acid sequence according to SEQ ID NO: 358. In some embodiments, the TL1A binding protein comprises a heavy chain variable region comprising an amino acid sequence having at least 85% sequence identity with an amino acid sequence according to SEQ ID NO: 340; and a light chain variable region comprising an amino acid sequence having at least 85% sequence identity with an amino acid sequence according to SEQ ID NO: 358. In some embodiments, the TL1A binding protein comprises a heavy chain variable region comprising an amino acid sequence having at least 90% sequence identity with an amino acid sequence according to SEQ ID NO: 340; and a light chain variable region comprising an amino acid sequence having at least 90% sequence identity with an amino acid sequence according to SEQ ID NO: 358. In some embodiments, the TL1A binding protein comprises a heavy chain variable region comprising an amino acid sequence having at least 95% sequence identity with an amino acid sequence according to SEQ ID NO: 340; and a light chain variable region comprising an amino acid sequence having at least 95% sequence identity with an amino acid sequence according to SEQ ID NO: 358. In some embodiments, the TL1A binding protein comprises a heavy chain variable region comprising an amino acid sequence having at least 96% sequence identity with an amino acid sequence according to SEQ ID NO: 340; and a light chain variable region comprising an amino acid sequence having at least 96% sequence identity with an amino acid sequence according to SEQ ID NO: 358. In some embodiments, the TL1A binding protein comprises a heavy chain variable region comprising an amino acid sequence having at least 97% sequence identity with an amino acid sequence according to SEQ ID NO: 340; and a light chain variable region comprising an amino acid sequence having at least 97% sequence identity with an amino acid sequence according to SEQ ID NO: 358. In some embodiments, the TL1A binding protein comprises a heavy chain variable region comprising an amino acid sequence having at least 98% sequence identity with an amino acid sequence according to SEQ ID NO: 340; and a light chain variable region comprising an amino acid sequence having at least 98% sequence identity with an amino acid sequence according to SEQ ID NO: 358. In some embodiments, the TL1A binding protein comprises a heavy chain variable region comprising an amino acid sequence having at least 99% sequence identity with an amino acid sequence according to SEQ ID NO: 340; and a light chain variable region comprising an amino acid sequence having at least 99% sequence identity with an amino acid sequence according SEQ ID NO: 358. In some embodiments, the TL1A binding protein comprises a heavy chain variable region comprising an amino acid sequence according to SEQ ID NO: 340; and a light chain variable region comprising an amino acid sequence according to SEQ ID NO: 358.

In some embodiments, the TL1A binding protein comprises a heavy chain variable region comprising an amino acid sequence having at least 80% sequence identity with an amino acid sequence according to SEQ ID NO: 341; and a light chain variable region comprising an amino acid sequence having at least 80% sequence identity with an amino acid sequence according to SEQ ID NO: 359. In some embodiments, the TL1A binding protein comprises a heavy chain variable region comprising an amino acid sequence having at least 85% sequence identity with an amino acid sequence according to SEQ ID NO: 341; and a light chain variable region comprising an amino acid sequence having at least 85% sequence identity with an amino acid sequence according to SEQ ID NO: 359. In some embodiments, the TL1A binding protein comprises a heavy chain variable region comprising an amino acid sequence having at least 90% sequence identity with an amino acid sequence according to SEQ ID NO: 341; and a light chain variable region comprising an amino acid sequence having at least 90% sequence identity with an amino acid sequence according to SEQ ID NO: 359. In some embodiments, the TL1A binding protein comprises a heavy chain variable region comprising an amino acid sequence having at least 95% sequence identity with an amino acid sequence according to SEQ ID NO: 341; and a light chain variable region comprising an amino acid sequence having at least 95% sequence identity with an amino acid sequence according to SEQ ID NO: 359. In some embodiments, the TL1A binding protein comprises a heavy chain variable region comprising an amino acid sequence having at least 96% sequence identity with an amino acid sequence according to SEQ ID NO: 341; and a light chain variable region comprising an amino acid sequence having at least 96% sequence identity with an amino acid sequence according to SEQ ID NO: 359. In some embodiments, the TL1A binding protein comprises a heavy chain variable region comprising an amino acid sequence having at least 97% sequence identity with an amino acid sequence according to SEQ ID NO: 341; and a light chain variable region comprising an amino acid sequence having at least 97% sequence identity with an amino acid sequence according to SEQ ID NO: 359. In some embodiments, the TL1A binding protein comprises a heavy chain variable region comprising an amino acid sequence having at least 98% sequence identity with an amino acid sequence according to SEQ ID NO: 341; and a light chain variable region comprising an amino acid sequence having at least 98% sequence identity with an amino acid sequence according to SEQ ID NO: 359. In some embodiments, the TL1A binding protein comprises a heavy chain variable region comprising an amino acid sequence having at least 99% sequence identity with an amino acid sequence according to SEQ ID NO: 341; and a light chain variable region comprising an amino acid sequence having at least 99% sequence identity with an amino acid sequence according SEQ ID NO: 359. In some embodiments, the TL1A binding protein comprises a heavy chain variable region comprising an amino acid sequence according to SEQ ID NO: 341; and a light chain variable region comprising an amino acid sequence according to SEQ ID NO: 359.

In some embodiments, the TL1A binding protein comprises a heavy chain variable region comprising an amino acid sequence having at least 80% sequence identity with an amino acid sequence according to SEQ ID NO: 342; and a light chain variable region comprising an amino acid sequence having at least 80% sequence identity with an amino acid sequence according to SEQ ID NO: 360. In some embodiments, the TL1A binding protein comprises a heavy chain variable region comprising an amino acid sequence having at least 85% sequence identity with an amino acid sequence according to SEQ ID NO: 342; and a light chain variable region comprising an amino acid sequence having at least 85% sequence identity with an amino acid sequence according to SEQ ID NO: 360. In some embodiments, the TL1A binding protein comprises a heavy chain variable region comprising an amino acid sequence having at least 90% sequence identity with an amino acid sequence according to SEQ ID NO: 342; and a light chain variable region comprising an amino acid sequence having at least 90% sequence identity with an amino acid sequence according to SEQ ID NO: 360. In some embodiments, the TL1A binding protein comprises a heavy chain variable region comprising an amino acid sequence having at least 95% sequence identity with an amino acid sequence according to SEQ ID NO: 342; and a light chain variable region comprising an amino acid sequence having at least 95% sequence identity with an amino acid sequence according to SEQ ID NO: 360. In some embodiments, the TL1A binding protein comprises a heavy chain variable region comprising an amino acid sequence having at least 96% sequence identity with an amino acid sequence according to SEQ ID NO: 342; and a light chain variable region comprising an amino acid sequence having at least 96% sequence identity with an amino acid sequence according to SEQ ID NO: 360. In some embodiments, the TL1A binding protein comprises a heavy chain variable region comprising an amino acid sequence having at least 97% sequence identity with an amino acid sequence according to SEQ ID NO: 342; and a light chain variable region comprising an amino acid sequence having at least 97% sequence identity with an amino acid sequence according to SEQ ID NO: 360. In some embodiments, the TL1A binding protein comprises a heavy chain variable region comprising an amino acid sequence having at least 98% sequence identity with an amino acid sequence according to SEQ ID NO: 342; and a light chain variable region comprising an amino acid sequence having at least 98% sequence identity with an amino acid sequence according to SEQ ID NO: 360. In some embodiments, the TL1A binding protein comprises a heavy chain variable region comprising an amino acid sequence having at least 99% sequence identity with an amino acid sequence according to SEQ ID NO: 342; and a light chain variable region comprising an amino acid sequence having at least 99% sequence identity with an amino acid sequence according SEQ ID NO: 360. In some embodiments, the TL1A binding protein comprises a heavy chain variable region comprising an amino acid sequence according to SEQ ID NO: 342; and a light chain variable region comprising an amino acid sequence according to SEQ ID NO: 360.

### Fc Modifications

Described herein are TL1A binding proteins comprising modified Fc regions. Unless otherwise specified herein, numbering of amino acid residues in the Fc region or constant region is according to the EU numbering system, also called the EU index, as described in Kabat et al, Sequences of Proteins of Immunological Interest, 5th Ed. Public Health Service, National Institutes of Health, Bethesda, MD, 1991.

In some embodiments, the TL1A binding proteins comprise a modified Fc comprising one or more modifications. In some embodiments, the one or more modifications are located in a Fc from IgG1 (e.g., human IgG1 (hIgG1). In some embodiments, the one or more modifications are located in a Fc from IgG4 (e.g., human IgG4 (hIgG4). In some embodiments, the one or more modifications are located in a Fc from IgG2. In some embodiments, the one or more modifications promote selective binding of Fc-gamma receptors.

Amino acid sequences of exemplary Fc sequences are provided in Table 3.

TABLE 3
Fc Sequences

| | SEQ | |
|---|---|---|
| | ID | |
| Name | NO | Fc Sequence |
| hIgG1 | 361 | ASTKGPSVFPLAPSSKSTSGGTAALGCLVKDYFPEPVTVSWNS |
| | | GALTSGVHTFPAVLQSSGLYSLSSVVTVPSSSLGTQTYICNVNH |
| | | KPSNTKVDKKVEPKSCDKTHTCPPCPAPELLGGPSVFLFPPKPK |
| | | DTLMISRTPEVTCVVVDVSHEDPEVKFNWYVDGVEVHNAKT |
| | | KPREEQYNSTYRVVSVLTVLHQDWLNGKEYKCKVSNKALPA |
| | | PIEKTISKAKGQPREPQVYTLPPSRDELTKNQVSLTCLVKGFYP |
| | | SDIAVEWESNGQPENNYKTTPPVLDSDGSFFLYSKLTVDKSRW |
| | | QQGNVFSCSVMHEALHNHYTQKSLSLSPG |
| hIgG4 | 362 | ASTKGPSVFPLAPCSRSTSESTAALGCLVKDYFPEPVTVSWNS |
| | | GALTSGVHTFPAVLQSSGLYSLSSVVTVPSSSLGTKTYTCNVD |
| | | HKPSNTKVDKRVESKYGPPCPSCPAPEFLGGPSVFLFPPKPKDT |
| | | LMISRTPEVTCVVVDVSQEDPEVQFNWYVDGVEVHNAKTKP |
| | | REEQFNSTYRVVSVLTVLHQDWLNGKEYKCKVSNKGLPSSIE |
| | | KTISKAKGQPREPQVYTLPPSQEEMTKNQVSLTCLVKGFYPSD |
| | | IAVEWESNGQPENNYKTTPPVLDSDGSFFLYSRLTVDKSRWQE |
| | | GNVFSCSVMHEALHNHYTQKSLSLSLGK |
| IgG2 | 363 | ASTKGPSVFPLAPCSRSTSESTAALGCLVKDYFPEPVTVSWNS |
| | | GALTSGVHTFPAVLQSSGLYSLSSVVTVPSSNFGTQTYTCNVD |
| | | HKPSNTKVDKTVERKCCVECPPCPAPPVAGPSVFLFPPKPKDT |
| | | LMISRTPEVTCVVVDVSHEDPEVQFNWYVDGVEVHNAKTKP |
| | | REEQFNSTFRVVSVLTVVHQDWLNGKEYKCKVSNKGLPAPIE |
| | | KTISKTKGQPREPQVYTLPPSREEMTKNQVSLTCLVKGFYPSDI |
| | | AVEWESNGQPENNYKTTPPMLDSDGSFFLYSKLTVDKSRWQQ |
| | | GNVFSCSVMHEALHNHYTQKSLSLSP |
| IgG4-SP | 364 | ASTKGPSVFPLAPCSRSTSESTAALGCLVKDYFPEPVTVSWNS |
| | | GALTSGVHTFPAVLQSSGLYSLSSVVTVPSSSLGTKTYTCNVD |
| | | HKPSNTKVDKRVESKYGPPCPPCPAPEFLGGPSVFLFPPKPKDT |
| | | LMISRTPEVTCVVVDVSQEDPEVQFNWYVDGVEVHNAKTKP |
| | | REEQFNSTYRVVSVLTVLHQDWLNGKEYKCKVSNKGLPSSIE |
| | | KTISKAKGQPREPQVYTLPPSQEEMTKNQVSLTCLVKGFYPSD |
| | | IAVEWESNGQPENNYKTTPPVLDSDGSFFLYSRLTVDKSRWQE |
| | | GNVFSCSVMHEALHNHYTQKSLSLSLGK |
| IgG4-SPLE | 365 | ASTKGPSVFPLAPCSRSTSESTAALGCLVKDYFPEPVTVSWNS |
| | | GALTSGVHTFPAVLQSSGLYSLSSVVTVPSSSLGTKTYTCNVD |
| | | HKPSNTKVDKRVESKYGPPCPPCPAPEELGGPSVFLFPPKPKDT |
| | | LMISRTPEVTCVVVDVSQEDPEVQFNWYVDGVEVHNAKTKP |
| | | REEQFNSTYRVVSVLTVLHQDWLNGKEYKCKVSNKGLPSSIE |
| | | KTISKAKGQPREPQVYTLPPSQEEMTKNQVSLTCLVKGFYPSD |
| | | IAVEWESNGQPENNYKTTPPVLDSDGSFFLYSRLTVDKSRWQE |
| | | GNVFSCSVMHEALHNHYTQKSLSLSLGK |
| hIgG1- | 366 | ASTKGPSVFPLAPSSKSTSGGTAALGCLVKDYFPEPVTVSWNS |
| N297A | | GALTSGVHTFPAVLQSSGLYSLSSVVTVPSSSLGTQTYICNVNH |
| | | KPSNTKVDKKVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVYTLPPSRDELTKNQVSLTCLVKGFYP |
| | | SDIAVEWESNGQPENNYKTTPPVLDSDGSFFLYSKLTVDKSRW |
| | | QQGNVFSCSVMHEALHNHYTQKSLSLSPG |
| hIgG1- | 367 | ASTKGPSVFPLAPSSKSTSGGTAALGCLVKDYFPEPVTVSWNS |
| D265A | | GALTSGVHTFPAVLQSSGLYSLSSVVTVPSSSLGTQTYICNVNH |
| | | KPSNTKVDKKVEPKSCDKTHTCPPCPAPELLGGPSVFLFPPKPK |
| | | DTLMISRTPEVTCVVVAVSHEDPEVKFNWYVDGVEVHNAKT |
| | | KPREEQYNSTYRVVSVLTVLHQDWLNGKEYKCKVSNKALPA |
| | | PIEKTISKAKGQPREPQVYTLPPSRDELTKNQVSLTCLVKGFYP |
| | | SDIAVEWESNGQPENNYKTTPPVLDSDGSFFLYSKLTVDKSRW |
| | | QQGNVFSCSVMHEALHNHYTQKSLSLSPG |
| hIgG1- | 368 | ASTKGPSVFPLAPSSKSTSGGTAALGCLVKDYFPEPVTVSWNS |
| LALA | | GALTSGVHTFPAVLQSSGLYSLSSVVTVPSSSLGTQTYICNVNH |
| | | KPSNTKVDKKVEPKSCDKTHTCPPCPAPEAAGGPSVFLFPPKP |
| | | KDTLMISRTPEVTCVVVDVSHEDPEVKFNWYVDGVEVHNAK |
| | | TKPREEQYNSTYRVVSVLTVLHQDWLNGKEYKCKVSNKALP |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVMHEALHNHYTQKSLSLSPG |
| hIgG1- | 369 | ASTKGPSVFPLAPSSKSTSGGTAALGCLVKDYFPEPVTVSWNS |
| LAGA | | GALTSGVHTFPAVLQSSGLYSLSSVVTVPSSSLGTQTYICNVNH |
| | | KPSNTKVDKKVEPKSCDKTHTCPPCPAPELAGAPSVFLFPPKP |
| | | KDTLMISRTPEVTCVVVDVSHEDPEVKFNWYVDGVEVHNAK |
| | | TKPREEQYNSTYRVVSVLTVLHQDWLNGKEYKCKVSNKALP |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVMHEALHNHYTQKSLSLSPG |
| hIgG1- | 370 | ASTKGPSVFPLAPSSKSTSGGTAALGCLVKDYFPEPVTVSWNS |
| LALAGA | | GALTSGVHTFPAVLQSSGLYSLSSVVTVPSSSLGTQTYICNVNH |
| | | KPSNTKVDKKVEPKSCDKTHTCPPCPAPEAAGAPSVFLFPPKP |
| | | KDTLMISRTPEVTCVVVDVSHEDPEVKFNWYVDGVEVHNAK |
| | | TKPREEQYNSTYRVVSVLTVLHQDWLNGKEYKCKVSNKALP |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVMHEALHNHYTQKSLSLSPG |
| hIgG1- | 371 | ASTKGPSVFPLAPSSKSTSGGTAALGCLVKDYFPEPVTVSWNS |
| LALAPG | | GALTSGVHTFPAVLQSSGLYSLSSVVTVPSSSLGTQTYICNVNH |
| | | KPSNTKVDKKVEPKSCDKTHTCPPCPAPEAAGGPSVFLFPPKP |
| | | KDTLMISRTPEVTCVVVDVSHEDPEVKFNWYVDGVEVHNAK |
| | | TKPREEQYNSTYRVVSVLTVLHQDWLNGKEYKCKVSNKALG |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVMHEALHNHYTQKSLSLSPG |
| hIgG1-YTE | 372 | ASTKGPSVFPLAPSSKSTSGGTAALGCLVKDYFPEPVTVSWNS |
| | | GALTSGVHTFPAVLQSSGLYSLSSVVTVPSSSLGTQTYICNVNH |
| | | KPSNTKVDKKVEPKSCDKTHTCPPCPAPELLGGPSVFLFPPKPK |
| | | DTLYITREPEVTCVVVDVSHEDPEVKFNWYVDGVEVHNAKT |
| | | KPREEQYNSTYRVVSVLTVLHQDWLNGKEYKCKVSNKALPA |
| | | PIEKTISKAKGQPREPQVYTLPPSRDELTKNQVSLTCLVKGFYP |
| | | SDIAVEWESNGQPENNYKTTPPVLDSDGSFFLYSKLTVDKSRW |
| | | QQGNVFSCSVMHEALHNHYTQKSLSLSPG |
| hIgG1- | 373 | ASTKGPSVFPLAPSSKSTSGGTAALGCLVKDYFPEPVTVSWNS |
| N297A/YTE | | GALTSGVHTFPAVLQSSGLYSLSSVVTVPSSSLGTQTYICNVNH |
| | | KPSNTKVDKKVEPKSCDKTHTCPPCPAPELLGGPSVFLFPPKPK |
| | | DTLYITREPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVYTLPPSRDELTKNQVSLTCLVKGFYP |
| | | SDIAVEWESNGQPENNYKTTPPVLDSDGSFFLYSKLTVDKSRW |
| | | QQGNVFSCSVMHEALHNHYTQKSLSLSPG |
| hIgG1- | 374 | ASTKGPSVFPLAPSSKSTSGGTAALGCLVKDYFPEPVTVSWNS |
| D265A/YTE | | GALTSGVHTFPAVLQSSGLYSLSSVVTVPSSSLGTQTYICNVNH |
| | | KPSNTKVDKKVEPKSCDKTHTCPPCPAPELLGGPSVFLFPPKPK |
| | | DTLYITREPEVTCVVVAVSHEDPEVKFNWYVDGVEVHNAKT |
| | | KPREEQYNSTYRVVSVLTVLHQDWLNGKEYKCKVSNKALPA |
| | | PIEKTISKAKGQPREPQVYTLPPSRDELTKNQVSLTCLVKGFYP |
| | | SDIAVEWESNGQPENNYKTTPPVLDSDGSFFLYSKLTVDKSRW |
| | | QQGNVFSCSVMHEALHNHYTQKSLSLSPG |
| hIgG1- | 375 | ASTKGPSVFPLAPSSKSTSGGTAALGCLVKDYFPEPVTVSWNS |
| LALA/YTE | | GALTSGVHTFPAVLQSSGLYSLSSVVTVPSSSLGTQTYICNVNH |
| | | KPSNTKVDKKVEPKSCDKTHTCPPCPAPEAAGGPSVFLFPPKP |
| | | KDTLYITREPEVTCVVVDVSHEDPEVKFNWYVDGVEVHNAK |
| | | TKPREEQYNSTYRVVSVLTVLHQDWLNGKEYKCKVSNKALP |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVMHEALHNHYTQKSLSLSPG |
| hIgG1- | 376 | ASTKGPSVFPLAPSSKSTSGGTAALGCLVKDYFPEPVTVSWNS |
| LAGA/YTE | | GALTSGVHTFPAVLQSSGLYSLSSVVTVPSSSLGTQTYICNVNH |
| | | KPSNTKVDKKVEPKSCDKTHTCPPCPAPELAGAPSVFLFPPKP |
| | | KDTLYITREPEVTCVVVDVSHEDPEVKFNWYVDGVEVHNAK |
| | | TKPREEQYNSTYRVVSVLTVLHQDWLNGKEYKCKVSNKALP |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVMHEALHNHYTQKSLSLSPG |
| hIgG1- | 377 | ASTKGPSVFPLAPSSKSTSGGTAALGCLVKDYFPEPVTVSWNS |
| LALAGA/ | | GALTSGVHTFPAVLQSSGLYSLSSVVTVPSSSLGTQTYICNVNH |
| YTE | | KPSNTKVDKKVEPKSCDKTHTCPPCPAPEAAGAPSVFLFPPKP |
| | | KDTLYITREPEVTCVVVDVSHEDPEVKFNWYVDGVEVHNAK |
| | | TKPREEQYNSTYRVVSVLTVLHQDWLNGKEYKCKVSNKALP |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVMHEALHNHYTQKSLSLSPG |
| hIgG1- | 378 | ASTKGPSVFPLAPSSKSTSGGTAALGCLVKDYFPEPVTVSWNS |
| LALAPG/ | | GALTSGVHTFPAVLQSSGLYSLSSVVTVPSSSLGTQTYICNVNH |
| YTE | | KPSNTKVDKKVEPKSCDKTHTCPPCPAPEAAGGPSVFLFPPKP |
| | | KDTLYITREPEVTCVVVDVSHEDPEVKFNWYVDGVEVHNAK |
| | | TKPREEQYNSTYRVVSVLTVLHQDWLNGKEYKCKVSNKALG |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVMHEALHNHYTQKSLSLSPG |
| hIgG1-LS | 379 | ASTKGPSVFPLAPSSKSTSGGTAALGCLVKDYFPEPVTVSWNS |
| | | GALTSGVHTFPAVLQSSGLYSLSSVVTVPSSSLGTQTYICNVNH |
| | | KPSNTKVDKKVEPKSCDKTHTCPPCPAPELLGGPSVFLFPPKPK |
| | | DTLMISRTPEVTCVVVDVSHEDPEVKFNWYVDGVEVHNAKT |
| | | KPREEQYNSTYRVVSVLTVLHQDWLNGKEYKCKVSNKALPA |
| | | PIEKTISKAKGQPREPQVYTLPPSRDELTKNQVSLTCLVKGFYP |
| | | SDIAVEWESNGQPENNYKTTPPVLDSDGSFFLYSKLTVDKSRW |
| | | QQGNVFSCSVLHEALHSHYTQKSLSLSPG |
| hIgG1- | 380 | ASTKGPSVFPLAPSSKSTSGGTAALGCLVKDYFPEPVTVSWNS |
| N297A/LS | | GALTSGVHTFPAVLQSSGLYSLSSVVTVPSSSLGTQTYICNVNH |
| | | KPSNTKVDKKVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVYTLPPSRDELTKNQVSLTCLVKGFYP |
| | | SDIAVEWESNGQPENNYKTTPPVLDSDGSFFLYSKLTVDKSRW |
| | | QQGNVFSCSVLHEALHSHYTQKSLSLSPG |
| hIgG1- | 381 | ASTKGPSVFPLAPSSKSTSGGTAALGCLVKDYFPEPVTVSWNS |
| D265A/LS | | GALTSGVHTFPAVLQSSGLYSLSSVVTVPSSSLGTQTYICNVNH |
| | | KPSNTKVDKKVEPKSCDKTHTCPPCPAPELLGGPSVFLFPPKPK |
| | | DTLMISRTPEVTCVVVAVSHEDPEVKFNWYVDGVEVHNAKT |
| | | KPREEQYNSTYRVVSVLTVLHQDWLNGKEYKCKVSNKALPA |
| | | PIEKTISKAKGQPREPQVYTLPPSRDELTKNQVSLTCLVKGFYP |
| | | SDIAVEWESNGQPENNYKTTPPVLDSDGSFFLYSKLTVDKSRW |
| | | QQGNVFSCSVLHEALHSHYTQKSLSLSPG |
| hIgG1- | 382 | ASTKGPSVFPLAPSSKSTSGGTAALGCLVKDYFPEPVTVSWNS |
| LALA/LS | | GALTSGVHTFPAVLQSSGLYSLSSVVTVPSSSLGTQTYICNVNH |
| | | KPSNTKVDKKVEPKSCDKTHTCPPCPAPEAAGGPSVFLFPPKP |
| | | KDTLMISRTPEVTCVVVDVSHEDPEVKFNWYVDGVEVHNAK |
| | | TKPREEQYNSTYRVVSVLTVLHQDWLNGKEYKCKVSNKALP |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVLHEALHSHYTQKSLSLSPG |
| hIgG1- | 383 | ASTKGPSVFPLAPSSKSTSGGTAALGCLVKDYFPEPVTVSWNS |
| LAGA/LS | | GALTSGVHTFPAVLQSSGLYSLSSVVTVPSSSLGTQTYICNVNH |
| | | KPSNTKVDKKVEPKSCDKTHTCPPCPAPELAGAPSVFLFPPKP |
| | | KDTLMISRTPEVTCVVVDVSHEDPEVKFNWYVDGVEVHNAK |
| | | TKPREEQYNSTYRVVSVLTVLHQDWLNGKEYKCKVSNKALP |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVLHEALHSHYTQKSLSLSPG |
| hIgG1- | 384 | ASTKGPSVFPLAPSSKSTSGGTAALGCLVKDYFPEPVTVSWNS |
| LALAGA/ | | GALTSGVHTFPAVLQSSGLYSLSSVVTVPSSSLGTQTYICNVNH |
| LS | | KPSNTKVDKKVEPKSCDKTHTCPPCPAPEAAGAPSVFLFPPKP |
| | | KDTLMISRTPEVTCVVVDVSHEDPEVKFNWYVDGVEVHNAK |
| | | TKPREEQYNSTYRVVSVLTVLHQDWLNGKEYKCKVSNKALP |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVLHEALHSHYTQKSLSLSPG |
| hIgG1- | 385 | ASTKGPSVFPLAPSSKSTSGGTAALGCLVKDYFPEPVTVSWNS |
| LALAPG/LS | | GALTSGVHTFPAVLQSSGLYSLSSVVTVPSSSLGTQTYICNVNH |
| | | KPSNTKVDKKVEPKSCDKTHTCPPCPAPEAAGGPSVFLFPPKP |
| | | KDTLMISRTPEVTCVVVDVSHEDPEVKFNWYVDGVEVHNAK |
| | | TKPREEQYNSTYRVVSVLTVLHQDWLNGKEYKCKVSNKALG |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVLHEALHSHYTQKSLSLSPG |
| hIgG1-DHS | 386 | ASTKGPSVFPLAPSSKSTSGGTAALGCLVKDYFPEPVTVSWNS |
| | | GALTSGVHTFPAVLQSSGLYSLSSVVTVPSSSLGTQTYICNVNH |
| | | KPSNTKVDKKVEPKSCDKTHTCPPCPAPELLGGPSVFLFPPKPK |
| | | DTLMISRTPEVTCVVVDVSHEDPEVKFNWYVDGVEVHNAKT |
| | | KPREEQYNSTYRVVSVLTVDHHDWLNGKEYKCKVSNKALPA |
| | | PIEKTISKAKGQPREPQVYTLPPSRDELTKNQVSLTCLVKGFYP |
| | | SDIAVEWESNGQPENNYKTTPPVLDSDGSFFLYSKLTVDKSRW |
| | | QQGNVFSCSVMHEALHSHYTQKSLSLSPG |
| hIgG1- | 387 | ASTKGPSVFPLAPSSKSTSGGTAALGCLVKDYFPEPVTVSWNS |
| N297A/DHS | | GALTSGVHTFPAVLQSSGLYSLSSVVTVPSSSLGTQTYICNVNH |
| | | KPSNTKVDKKVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVDHHDWLNGKEYKCKVSNKALPA |
| | | PIEKTISKAKGQPREPQVYTLPPSRDELTKNQVSLTCLVKGFYP |
| | | SDIAVEWESNGQPENNYKTTPPVLDSDGSFFLYSKLTVDKSRW |
| | | QQGNVFSCSVMHEALHSHYTQKSLSLSPG |
| hIgG1- | 388 | ASTKGPSVFPLAPSSKSTSGGTAALGCLVKDYFPEPVTVSWNS |
| D265A/DHS | | GALTSGVHTFPAVLQSSGLYSLSSVVTVPSSSLGTQTYICNVNH |
| | | KPSNTKVDKKVEPKSCDKTHTCPPCPAPELLGGPSVFLFPPKPK |
| | | DTLMISRTPEVTCVVVAVSHEDPEVKFNWYVDGVEVHNAKT |
| | | KPREEQYNSTYRVVSVLTVDHHDWLNGKEYKCKVSNKALPA |
| | | PIEKTISKAKGQPREPQVYTLPPSRDELTKNQVSLTCLVKGFYP |
| | | SDIAVEWESNGQPENNYKTTPPVLDSDGSFFLYSKLTVDKSRW |
| | | QQGNVFSCSVMHEALHSHYTQKSLSLSPG |
| hIgG1- | 389 | ASTKGPSVFPLAPSSKSTSGGTAALGCLVKDYFPEPVTVSWNS |
| LALA/DHS | | GALTSGVHTFPAVLQSSGLYSLSSVVTVPSSSLGTQTYICNVNH |
| | | KPSNTKVDKKVEPKSCDKTHTCPPCPAPEAAGGPSVFLFPPKP |
| | | KDTLMISRTPEVTCVVVDVSHEDPEVKFNWYVDGVEVHNAK |
| | | TKPREEQYNSTYRVVSVLTVDHHDWLNGKEYKCKVSNKALP |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVMHEALHSHYTQKSLSLSPG |
| hIgG1- | 390 | ASTKGPSVFPLAPSSKSTSGGTAALGCLVKDYFPEPVTVSWNS |
| LAGA/DHS | | GALTSGVHTFPAVLQSSGLYSLSSVVTVPSSSLGTQTYICNVNH |
| | | KPSNTKVDKKVEPKSCDKTHTCPPCPAPELAGAPSVFLFPPKP |
| | | KDTLMISRTPEVTCVVVDVSHEDPEVKFNWYVDGVEVHNAK |
| | | TKPREEQYNSTYRVVSVLTVDHHDWLNGKEYKCKVSNKALP |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVMHEALHSHYTQKSLSLSPG |
| hIgG1- | 391 | ASTKGPSVFPLAPSSKSTSGGTAALGCLVKDYFPEPVTVSWNS |
| LALAGA/ | | GALTSGVHTFPAVLQSSGLYSLSSVVTVPSSSLGTQTYICNVNH |
| DHS | | KPSNTKVDKKVEPKSCDKTHTCPPCPAPEAAGAPSVFLFPPKP |
| | | KDTLMISRTPEVTCVVVDVSHEDPEVKFNWYVDGVEVHNAK |
| | | TKPREEQYNSTYRVVSVLTVDHHDWLNGKEYKCKVSNKALP |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVMHEALHSHYTQKSLSLSPG |
| hIgG1- | 392 | ASTKGPSVFPLAPSSKSTSGGTAALGCLVKDYFPEPVTVSWNS |
| LALAPG/ | | GALTSGVHTFPAVLQSSGLYSLSSVVTVPSSSLGTQTYICNVNH |
| DHS | | KPSNTKVDKKVEPKSCDKTHTCPPCPAPEAAGGPSVFLFPPKP |
| | | KDTLMISRTPEVTCVVVDVSHEDPEVKFNWYVDGVEVHNAK |
| | | TKPREEQYNSTYRVVSVLTVDHHDWLNGKEYKCKVSNKALG |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVMHEALHSHYTQKSLSLSPG |
| hIgG4-YTE | 393 | ASTKGPSVFPLAPCSRSTSESTAALGCLVKDYFPEPVTVSWNS |
| | | GALTSGVHTFPAVLQSSGLYSLSSVVTVPSSSLGTKTYTCNVD |
| | | HKPSNTKVDKRVESKYGPPCPSCPAPEFLGGPSVFLFPPKPKDT |
| | | LYITREPEVTCVVVDVSQEDPEVQFNWYVDGVEVHNAKTKPR |
| | | EEQFNSTYRVVSVLTVLHQDWLNGKEYKCKVSNKGLPSSIEK |
| | | TISKAKGQPREPQVYTLPPSQEEMTKNQVSLTCLVKGFYPSDI |
| | | AVEWESNGQPENNYKTTPPVLDSDGSFFLYSRLTVDKSRWQE |
| | | GNVFSCSVMHEALHNHYTQKSLSLSLGK |
| hIgG4- | 394 | ASTKGPSVFPLAPCSRSTSESTAALGCLVKDYFPEPVTVSWNS |
| SP/YTE | | GALTSGVHTFPAVLQSSGLYSLSSVVTVPSSSLGTKTYTCNVD |
| | | HKPSNTKVDKRVESKYGPPCPPCPAPEFLGGPSVFLFPPKPKDT |
| | | LYITREPEVTCVVVDVSQEDPEVQFNWYVDGVEVHNAKTKPR |
| | | EEQFNSTYRVVSVLTVLHQDWLNGKEYKCKVSNKGLPSSIEK |
| | | TISKAKGQPREPQVYTLPPSQEEMTKNQVSLTCLVKGFYPSDI |
| | | AVEWESNGQPENNYKTTPPVLDSDGSFFLYSRLTVDKSRWQE |
| | | GNVFSCSVMHEALHNHYTQKSLSLSLGK |
| hIgG4- | 395 | ASTKGPSVFPLAPCSRSTSESTAALGCLVKDYFPEPVTVSWNS |
| SPLE/YTE | | GALTSGVHTFPAVLQSSGLYSLSSVVTVPSSSLGTKTYTCNVD |
| | | HKPSNTKVDKRVESKYGPPCPPCPAPEELGGPSVFLFPPKPKDT |
| | | LYITREPEVTCVVVDVSQEDPEVQFNWYVDGVEVHNAKTKPR |
| | | EEQFNSTYRVVSVLTVLHQDWLNGKEYKCKVSNKGLPSSIEK |
| | | TISKAKGQPREPQVYTLPPSQEEMTKNQVSLTCLVKGFYPSDI |
| | | AVEWESNGQPENNYKTTPPVLDSDGSFFLYSRLTVDKSRWQE |
| | | GNVFSCSVMHEALHNHYTQKSLSLSLGK |
| hIgG4-LS | 396 | ASTKGPSVFPLAPCSRSTSESTAALGCLVKDYFPEPVTVSWNS |
| | | GALTSGVHTFPAVLQSSGLYSLSSVVTVPSSSLGTKTYTCNVD |
| | | HKPSNTKVDKRVESKYGPPCPSCPAPEFLGGPSVFLFPPKPKDT |
| | | LMISRTPEVTCVVVDVSQEDPEVQFNWYVDGVEVHNAKTKP |
| | | REEQFNSTYRVVSVLTVLHQDWLNGKEYKCKVSNKGLPSSIE |
| | | KTISKAKGQPREPQVYTLPPSQEEMTKNQVSLTCLVKGFYPSD |
| | | IAVEWESNGQPENNYKTTPPVLDSDGSFFLYSRLTVDKSRWQE |
| | | GNVFSCSVLHEALHSYTQKSLSLSLGK |
| hIgG4-SP/ | 397 | ASTKGPSVFPLAPCSRSTSESTAALGCLVKDYFPEPVTVSWNS |
| LS | | GALTSGVHTFPAVLQSSGLYSLSSVVTVPSSSLGTKTYTCNVD |
| | | HKPSNTKVDKRVESKYGPPCPPCPAPEFLGGPSVFLFPPKPKDT |
| | | LMISRTPEVTCVVVDVSQEDPEVQFNWYVDGVEVHNAKTKP |
| | | REEQFNSTYRVVSVLTVLHQDWLNGKEYKCKVSNKGLPSSIE |
| | | KTISKAKGQPREPQVYTLPPSQEEMTKNQVSLTCLVKGFYPSD |
| | | IAVEWESNGQPENNYKTTPPVLDSDGSFFLYSRLTVDKSRWQE |
| | | GNVFSCSVLHEALHSHYTQKSLSLSLGK |
| hIgG4- | 398 | ASTKGPSVFPLAPCSRSTSESTAALGCLVKDYFPEPVTVSWNS |
| SPLE/LS | | GALTSGVHTFPAVLQSSGLYSLSSVVTVPSSSLGTKTYTCNVD |
| | | HKPSNTKVDKRVESKYGPPCPPCPAPEELGGPSVFLFPPKPKDT |
| | | LMISRTPEVTCVVVDVSQEDPEVQFNWYVDGVEVHNAKTKP |
| | | REEQFNSTYRVVSVLTVLHQDWLNGKEYKCKVSNKGLPSSIE |
| | | KTISKAKGQPREPQVYTLPPSQEEMTKNQVSLTCLVKGFYPSD |
| | | IAVEWESNGQPENNYKTTPPVLDSDGSFFLYSRLTVDKSRWQE |
| | | GNVFSCSVLHEALHSHYTQKSLSLSLGK |
| hIgG4-DHS | 399 | ASTKGPSVFPLAPCSRSTSESTAALGCLVKDYFPEPVTVSWNS |
| | | GALTSGVHTFPAVLQSSGLYSLSSVVTVPSSSLGTKTYTCNVD |
| | | HKPSNTKVDKRVESKYGPPCPSCPAPEFLGGPSVFLFPPKPKDT |
| | | LMISRTPEVTCVVVDVSQEDPEVQFNWYVDGVEVHNAKTKP |
| | | REEQFNSTYRVVSVLTVDHHDWLNGKEYKCKVSNKGLPSSIE |
| | | KTISKAKGQPREPQVYTLPPSQEEMTKNQVSLTCLVKGFYPSD |
| | | IAVEWESNGQPENNYKTTPPVLDSDGSFFLYSRLTVDKSRWQE |
| | | GNVFSCSVMHEALHSHYTQKSLSLSLGK |
| hIgG4- | 400 | ASTKGPSVFPLAPCSRSTSESTAALGCLVKDYFPEPVTVSWNS |
| SP/DHS | | GALTSGVHTFPAVLQSSGLYSLSSVVTVPSSSLGTKTYTCNVD |
| | | HKPSNTKVDKRVESKYGPPCPPCPAPEFLGGPSVFLFPPKPKDT |
| | | LMISRTPEVTCVVVDVSQEDPEVQFNWYVDGVEVHNAKTKP |
| | | REEQFNSTYRVVSVLTVDHHDWLNGKEYKCKVSNKGLPSSIE |
| | | KTISKAKGQPREPQVYTLPPSQEEMTKNQVSLTCLVKGFYPSD |
| | | IAVEWESNGQPENNYKTTPPVLDSDGSFFLYSRLTVDKSRWQE |
| | | GNVFSCSVMHEALHSHYTQKSLSLSLGK |
| hIgG4- | 401 | ASTKGPSVFPLAPCSRSTSESTAALGCLVKDYFPEPVTVSWNS |
| SPLE/DHS | | GALTSGVHTFPAVLQSSGLYSLSSVVTVPSSSLGTKTYTCNVD |
| | | HKPSNTKVDKRVESKYGPPCPPCPAPEELGGPSVFLFPPKPKDT |
| | | LMISRTPEVTCVVVDVSQEDPEVQFNWYVDGVEVHNAKTKP |
| | | REEQFNSTYRVVSVLTVDHHDWLNGKEYKCKVSNKGLPSSIE |
| | | KTISKAKGQPREPQVYTLPPSQEEMTKNQVSLTCLVKGFYPSD |
| | | IAVEWESNGQPENNYKTTPPVLDSDGSFFLYSRLTVDKSRWQE |
| | | GNVFSCSVMHEALHSHYTQKSLSLSLGK |
| hIgG2-YTE | 402 | ASTKGPSVFPLAPCSRSTSESTAALGCLVKDYFPEPVTVSWNS |
| | | GALTSGVHTFPAVLQSSGLYSLSSVVTVPSSNFGTQTYTCNVD |
| | | HKPSNTKVDKTVERKCCVECPPCPAPPVAGPSVFLFPPKPKDT |
| | | LYITREPEVTCVVVDVSHEDPEVQFNWYVDGVEVHNAKTKPR |
| | | EEQFNSTFRVVSVLTVVHQDWLNGKEYKCKVSNKGLPAPIEK |
| | | TISKTKGQPREPQVYTLPPSREEMTKNQVSLTCLVKGFYPSDIA |
| | | VEWESNGQPENNYKTTPPMLDSDGSFFLYSKLTVDKSRWQQG |
| | | NVFSCSVLHEALHSHYTQKSLSLSP |
| hIgG2-LS | 403 | ASTKGPSVFPLAPCSRSTSESTAALGCLVKDYFPEPVTVSWNS |
| | | GALTSGVHTFPAVLQSSGLYSLSSVVTVPSSNFGTQTYTCNVD |
| | | HKPSNTKVDKTVERKCCVECPPCPAPPVAGPSVFLFPPKPKDT |
| | | LMISRTPEVTCVVVDVSHEDPEVQFNWYVDGVEVHNAKTKP |
| | | REEQFNSTFRVVSVLTVVHQDWLNGKEYKCKVSNKGLPAPIE |
| | | KTISKTKGQPREPQVYTLPPSREEMTKNQVSLTCLVKGFYPSDI |
| | | AVEWESNGQPENNYKTTPPMLDSDGSFFLYSKLTVDKSRWQQ |
| | | GNVFSCSVMHEALHNHYTQKSLSLSP |
| hIgG2-DHS | 404 | ASTKGPSVFPLAPCSRSTSESTAALGCLVKDYFPEPVTVSWNS |
| | | GALTSGVHTFPAVLQSSGLYSLSSVVTVPSSNFGTQTYTCNVD |
| | | HKPSNTKVDKTVERKCCVECPPCPAPPVAGPSVFLFPPKPKDT |
| | | LMISRTPEVTCVVVDVSHEDPEVQFNWYVDGVEVHNAKTKP |
| | | REEQFNSTFRVVSVLTVDHHDWLNGKEYKCKVSNKGLPAPIE |
| | | KTISKTKGQPREPQVYTLPPSREEMTKNQVSLTCLVKGFYPSDI |
| | | AVEWESNGQPENNYKTTPPMLDSDGSFFLYSKLTVDKSRWQQ |
| | | GNVFSCSVMHEALHSHYTQKSLSLSP |
| IgG4-SP | 405 | ASTKGPSVFPLAPCSRSTSESTAALGCLVKDYFPEPVTVSWNS |
| | | GALTSGVHTFPAVLQSSGLYSLSSVVTVPSSSLGTKTYTCNVD |
| | | HKPSNTKVDKRVESKYGPPCPPCPAPEFLGGPSVFLFPPKPKDT |
| | | LMISRTPEVTCVVVDVSQEDPEVQFNWYVDGVEVHNAKTKP |
| | | REEQFNSTYRVVSVLTVLHQDWLNGKEYKCKVSNKGLPSSIE |
| | | KTISKAKGQPREPQVYTLPPSQEEMTKNQVSLTCLVKGFYPSD |
| | | IAVEWESNGQPENNYKTTPPVLDSDGSFFLYSRLTVDKSRWQE |
| | | GNVFSCSVMHEALHNHYTQKSLSLSLGK |
| hIgG1-LA | 406 | ASTKGPSVFPLAPSSKSTSGGTAALGCLVKDYFPEPVTVSWNS |
| | | GALTSGVHTFPAVLQSSGLYSLSSVVTVPSSSLGTQTYICNVNH |
| | | KPSNTKVDKKVEPKSCDKTHTCPPCPAPELLGGPSVFLFPPKPK |
| | | DTLMISRTPEVTCVVVDVSHEDPEVKFNWYVDGVEVHNAKT |
| | | KPREEQYNSTYRVVSVLTVLHQDWLNGKEYKCKVSNKALPA |
| | | PIEKTISKAKGQPREPQVYTLPPSRDELTKNQVSLTCLVKGFYP |
| | | SDIAVEWESNGQPENNYKTTPPVLDSDGSFFLYSKLTVDKSRW |
| | | QQGNVFSCSVLHEALHAHYTQKSLSLSPG |
| hIgG1- | 407 | ASTKGPSVFPLAPSSKSTSGGTAALGCLVKDYFPEPVTVSWNS |
| N297A/LA | | GALTSGVHTFPAVLQSSGLYSLSSVVTVPSSSLGTQTYICNVNH |
| | | KPSNTKVDKKVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVYTLPPSRDELTKNQVSLTCLVKGFYP |
| | | SDIAVEWESNGQPENNYKTTPPVLDSDGSFFLYSKLTVDKSRW |
| | | QQGNVFSCSVLHEALHAHYTQKSLSLSPG |
| hIgG1- | 408 | ASTKGPSVFPLAPSSKSTSGGTAALGCLVKDYFPEPVTVSWNS |
| D265A/LA | | GALTSGVHTFPAVLQSSGLYSLSSVVTVPSSSLGTQTYICNVNH |
| | | KPSNTKVDKKVEPKSCDKTHTCPPCPAPELLGGPSVFLFPPKPK |
| | | DTLMISRTPEVTCVVVAVSHEDPEVKFNWYVDGVEVHNAKT |
| | | KPREEQYNSTYRVVSVLTVLHQDWLNGKEYKCKVSNKALPA |
| | | PIEKTISKAKGQPREPQVYTLPPSRDELTKNQVSLTCLVKGFYP |
| | | SDIAVEWESNGQPENNYKTTPPVLDSDGSFFLYSKLTVDKSRW |
| | | QQGNVFSCSVLHEALHAHYTQKSLSLSPG |
| hIgG1- | 409 | ASTKGPSVFPLAPSSKSTSGGTAALGCLVKDYFPEPVTVSWNS |
| LALA/LA | | GALTSGVHTFPAVLQSSGLYSLSSVVTVPSSSLGTQTYICNVNH |
| | | KPSNTKVDKKVEPKSCDKTHTCPPCPAPEAAGGPSVFLFPPKP |
| | | KDTLMISRTPEVTCVVVDVSHEDPEVKFNWYVDGVEVHNAK |
| | | TKPREEQYNSTYRVVSVLTVLHQDWLNGKEYKCKVSNKALP |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVLHEALHAHYTQKSLSLSPG |
| hIgG1- | 410 | ASTKGPSVFPLAPSSKSTSGGTAALGCLVKDYFPEPVTVSWNS |
| LAGA/LA | | GALTSGVHTFPAVLQSSGLYSLSSVVTVPSSSLGTQTYICNVNH |
| | | KPSNTKVDKKVEPKSCDKTHTCPPCPAPELAGAPSVFLFPPKP |
| | | KDTLMISRTPEVTCVVVDVSHEDPEVKFNWYVDGVEVHNAK |
| | | TKPREEQYNSTYRVVSVLTVLHQDWLNGKEYKCKVSNKALP |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVLHEALHAHYTQKSLSLSPG |
| hIgG1- | 411 | ASTKGPSVFPLAPSSKSTSGGTAALGCLVKDYFPEPVTVSWNS |
| LALAGA/ | | GALTSGVHTFPAVLQSSGLYSLSSVVTVPSSSLGTQTYICNVNH |
| LA | | KPSNTKVDKKVEPKSCDKTHTCPPCPAPEAAGAPSVFLFPPKP |
| | | KDTLMISRTPEVTCVVVDVSHEDPEVKFNWYVDGVEVHNAK |
| | | TKPREEQYNSTYRVVSVLTVLHQDWLNGKEYKCKVSNKALP |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVLHEALHAHYTQKSLSLSPG |
| hIgG1- | 412 | ASTKGPSVFPLAPSSKSTSGGTAALGCLVKDYFPEPVTVSWNS |
| LALAPG/ | | GALTSGVHTFPAVLQSSGLYSLSSVVTVPSSSLGTQTYICNVNH |
| LA | | KPSNTKVDKKVEPKSCDKTHTCPPCPAPEAAGGPSVFLFPPKP |
| | | KDTLMISRTPEVTCVVVDVSHEDPEVKFNWYVDGVEVHNAK |
| | | TKPREEQYNSTYRVVSVLTVLHQDWLNGKEYKCKVSNKALG |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVLHEALHAHYTQKSLSLSPG |
| hIgG1- | 413 | ASTKGPSVFPLAPSSKSTSGGTAALGCLVKDYFPEPVTVSWNS |
| N434A | | GALTSGVHTFPAVLQSSGLYSLSSVVTVPSSSLGTQTYICNVNH |
| | | KPSNTKVDKKVEPKSCDKTHTCPPCPAPELLGGPSVFLFPPKPK |
| | | DTLMISRTPEVTCVVVDVSHEDPEVKFNWYVDGVEVHNAKT |
| | | KPREEQYNSTYRVVSVLTVLHQDWLNGKEYKCKVSNKALPA |
| | | PIEKTISKAKGQPREPQVYTLPPSRDELTKNQVSLTCLVKGFYP |
| | | SDIAVEWESNGQPENNYKTTPPVLDSDGSFFLYSKLTVDKSRW |
| | | QQGNVFSCSVMHEALHAHYTQKSLSLSPG |
| hIgG1- | 414 | ASTKGPSVFPLAPSSKSTSGGTAALGCLVKDYFPEPVTVSWNS |
| N297A/ | | GALTSGVHTFPAVLQSSGLYSLSSVVTVPSSSLGTQTYICNVNH |
| N434A | | KPSNTKVDKKVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVYTLPPSRDELTKNQVSLTCLVKGFYP |
| | | SDIAVEWESNGQPENNYKTTPPVLDSDGSFFLYSKLTVDKSRW |
| | | QQGNVFSCSVMHEALHAHYTQKSLSLSPG |
| hIgG1- | 415 | ASTKGPSVFPLAPSSKSTSGGTAALGCLVKDYFPEPVTVSWNS |
| D265A/ | | GALTSGVHTFPAVLQSSGLYSLSSVVTVPSSSLGTQTYICNVNH |
| N434A | | KPSNTKVDKKVEPKSCDKTHTCPPCPAPELLGGPSVFLFPPKPK |
| | | DTLMISRTPEVTCVVVAVSHEDPEVKFNWYVDGVEVHNAKT |
| | | KPREEQYNSTYRVVSVLTVLHQDWLNGKEYKCKVSNKALPA |
| | | PIEKTISKAKGQPREPQVYTLPPSRDELTKNQVSLTCLVKGFYP |
| | | SDIAVEWESNGQPENNYKTTPPVLDSDGSFFLYSKLTVDKSRW |
| | | QQGNVFSCSVMHEALHAHYTQKSLSLSPG |
| hIgG1- | 416 | ASTKGPSVFPLAPSSKSTSGGTAALGCLVKDYFPEPVTVSWNS |
| LALA/ | | GALTSGVHTFPAVLQSSGLYSLSSVVTVPSSSLGTQTYICNVNH |
| N434A | | KPSNTKVDKKVEPKSCDKTHTCPPCPAPEAAGGPSVFLFPPKP |
| | | KDTLMISRTPEVTCVVVDVSHEDPEVKFNWYVDGVEVHNAK |
| | | TKPREEQYNSTYRVVSVLTVLHQDWLNGKEYKCKVSNKALP |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVMHEALHAHYTQKSLSLSPG |
| hIgG1- | 417 | ASTKGPSVFPLAPSSKSTSGGTAALGCLVKDYFPEPVTVSWNS |
| LAGA/ | | GALTSGVHTFPAVLQSSGLYSLSSVVTVPSSSLGTQTYICNVNH |
| N434A | | KPSNTKVDKKVEPKSCDKTHTCPPCPAPELAGAPSVFLFPPKP |
| | | KDTLMISRTPEVTCVVVDVSHEDPEVKFNWYVDGVEVHNAK |
| | | TKPREEQYNSTYRVVSVLTVLHQDWLNGKEYKCKVSNKALP |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVMHEALHAHYTQKSLSLSPG |
| hIgG1- | 418 | ASTKGPSVFPLAPSSKSTSGGTAALGCLVKDYFPEPVTVSWNS |
| LALAGA/ | | GALTSGVHTFPAVLQSSGLYSLSSVVTVPSSSLGTQTYICNVNH |
| N434A | | KPSNTKVDKKVEPKSCDKTHTCPPCPAPEAAGAPSVFLFPPKP |
| | | KDTLMISRTPEVTCVVVDVSHEDPEVKFNWYVDGVEVHNAK |
| | | TKPREEQYNSTYRVVSVLTVLHQDWLNGKEYKCKVSNKALP |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVMHEALHAHYTQKSLSLSPG |
| hIgG1- | 419 | ASTKGPSVFPLAPSSKSTSGGTAALGCLVKDYFPEPVTVSWNS |
| LALAPG/ | | GALTSGVHTFPAVLQSSGLYSLSSVVTVPSSSLGTQTYICNVNH |
| N434A | | KPSNTKVDKKVEPKSCDKTHTCPPCPAPEAAGGPSVFLFPPKP |
| | | KDTLMISRTPEVTCVVVDVSHEDPEVKFNWYVDGVEVHNAK |
| | | TKPREEQYNSTYRVVSVLTVLHQDWLNGKEYKCKVSNKALG |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVMHEALHAHYTQKSLSLSPG |
| hIgG1- | 420 | ASTKGPSVFPLAPSSKSTSGGTAALGCLVKDYFPEPVTVSWNS |
| N434W | | GALTSGVHTFPAVLQSSGLYSLSSVVTVPSSSLGTQTYICNVNH |
| | | KPSNTKVDKKVEPKSCDKTHTCPPCPAPELLGGPSVFLFPPKPK |
| | | DTLMISRTPEVTCVVVDVSHEDPEVKFNWYVDGVEVHNAKT |
| | | KPREEQYNSTYRVVSVLTVLHQDWLNGKEYKCKVSNKALPA |
| | | PIEKTISKAKGQPREPQVYTLPPSRDELTKNQVSLTCLVKGFYP |
| | | SDIAVEWESNGQPENNYKTTPPVLDSDGSFFLYSKLTVDKSRW |
| | | QQGNVFSCSVMHEALHWHYTQKSLSLSPG |
| hIgG1- | 421 | ASTKGPSVFPLAPSSKSTSGGTAALGCLVKDYFPEPVTVSWNS |
| N297A/ | | GALTSGVHTFPAVLQSSGLYSLSSVVTVPSSSLGTQTYICNVNH |
| N434W | | KPSNTKVDKKVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVYTLPPSRDELTKNQVSLTCLVKGFYP |
| | | SDIAVEWESNGQPENNYKTTPPVLDSDGSFFLYSKLTVDKSRW |
| | | QQGNVFSCSVMHEALHWHYTQKSLSLSPG |
| hIgG1- | 422 | ASTKGPSVFPLAPSSKSTSGGTAALGCLVKDYFPEPVTVSWNS |
| D265A/ | | GALTSGVHTFPAVLQSSGLYSLSSVVTVPSSSLGTQTYICNVNH |
| N434W | | KPSNTKVDKKVEPKSCDKTHTCPPCPAPELLGGPSVFLFPPKPK |
| | | DTLMISRTPEVTCVVVAVSHEDPEVKFNWYVDGVEVHNAKT |
| | | KPREEQYNSTYRVVSVLTVLHQDWLNGKEYKCKVSNKALPA |
| | | PIEKTISKAKGQPREPQVYTLPPSRDELTKNQVSLTCLVKGFYP |
| | | SDIAVEWESNGQPENNYKTTPPVLDSDGSFFLYSKLTVDKSRW |
| | | QQGNVFSCSVMHEALHWHYTQKSLSLSPG |
| hIgG1- | 423 | ASTKGPSVFPLAPSSKSTSGGTAALGCLVKDYFPEPVTVSWNS |
| LALA/ | | GALTSGVHTFPAVLQSSGLYSLSSVVTVPSSSLGTQTYICNVNH |
| N434W | | KPSNTKVDKKVEPKSCDKTHTCPPCPAPEAAGGPSVFLFPPKP |
| | | KDTLMISRTPEVTCVVVDVSHEDPEVKFNWYVDGVEVHNAK |
| | | TKPREEQYNSTYRVVSVLTVLHQDWLNGKEYKCKVSNKALP |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVMHEALHWHYTQKSLSLSPG |
| hIgG1- | 424 | ASTKGPSVFPLAPSSKSTSGGTAALGCLVKDYFPEPVTVSWNS |
| LAGA/ | | GALTSGVHTFPAVLQSSGLYSLSSVVTVPSSSLGTQTYICNVNH |
| N434W | | KPSNTKVDKKVEPKSCDKTHTCPPCPAPELAGAPSVFLFPPKP |
| | | KDTLMISRTPEVTCVVVDVSHEDPEVKFNWYVDGVEVHNAK |
| | | TKPREEQYNSTYRVVSVLTVLHQDWLNGKEYKCKVSNKALP |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVMHEALHWHYTQKSLSLSPG |
| hIgG1- | 425 | ASTKGPSVFPLAPSSKSTSGGTAALGCLVKDYFPEPVTVSWNS |
| LALAGA/ | | GALTSGVHTFPAVLQSSGLYSLSSVVTVPSSSLGTQTYICNVNH |
| N434W | | KPSNTKVDKKVEPKSCDKTHTCPPCPAPEAAGAPSVFLFPPKP |
| | | KDTLMISRTPEVTCVVVDVSHEDPEVKFNWYVDGVEVHNAK |
| | | TKPREEQYNSTYRVVSVLTVLHQDWLNGKEYKCKVSNKALP |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVMHEALHWHYTQKSLSLSPG |
| hIgG1- | 426 | ASTKGPSVFPLAPSSKSTSGGTAALGCLVKDYFPEPVTVSWNS |
| LALAPG/ | | GALTSGVHTFPAVLQSSGLYSLSSVVTVPSSSLGTQTYICNVNH |
| N434W | | KPSNTKVDKKVEPKSCDKTHTCPPCPAPEAAGGPSVFLFPPKP |
| | | KDTLMISRTPEVTCVVVDVSHEDPEVKFNWYVDGVEVHNAK |
| | | TKPREEQYNSTYRVVSVLTVLHQDWLNGKEYKCKVSNKALG |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVMHEALHWHYTQKSLSLSPG |
| hIgG1/DQ | 427 | ASTKGPSVFPLAPSSKSTSGGTAALGCLVKDYFPEPVTVSWNS |
| | | GALTSGVHTFPAVLQSSGLYSLSSVVTVPSSSLGTQTYICNVNH |
| | | KPSNTKVDKKVEPKSCDKTHTCPPCPAPELLGGPSVFLFPPKPK |
| | | DTLMISRDPEVTCVVVDVSHEDPEVKFNWYVDGVEVHNAKT |
| | | KPREEQYNSTYRVVSVLQVLHQDWLNGKEYKCKVSNKALPA |
| | | PIEKTISKAKGQPREPQVYTLPPSRDELTKNQVSLTCLVKGFYP |
| | | SDIAVEWESNGQPENNYKTTPPVLDSDGSFFLYSKLTVDKSRW |
| | | QQGNVFSCSVMHEALHNHYTQKSLSLSPG |
| hIgG1- | 428 | ASTKGPSVFPLAPSSKSTSGGTAALGCLVKDYFPEPVTVSWNS |
| N297A/DQ | | GALTSGVHTFPAVLQSSGLYSLSSVVTVPSSSLGTQTYICNVNH |
| | | KPSNTKVDKKVEPKSCDKTHTCPPCPAPELLGGPSVFLFPPKPK |
| | | DTLMISRDPEVTCVVVDVSHEDPEVKFNWYVDGVEVHNAKT |
| | | KPREEQYASTYRVVSVLQVLHQDWLNGKEYKCKVSNKALPA |
| | | PIEKTISKAKGQPREPQVYTLPPSRDELTKNQVSLTCLVKGFYP |
| | | SDIAVEWESNGQPENNYKTTPPVLDSDGSFFLYSKLTVDKSRW |
| | | QQGNVFSCSVMHEALHNHYTQKSLSLSPG |
| hIgG1- | 429 | ASTKGPSVFPLAPSSKSTSGGTAALGCLVKDYFPEPVTVSWNS |
| D265A/DQ | | GALTSGVHTFPAVLQSSGLYSLSSVVTVPSSSLGTQTYICNVNH |
| | | KPSNTKVDKKVEPKSCDKTHTCPPCPAPELLGGPSVFLFPPKPK |
| | | DTLMISRDPEVTCVVVAVSHEDPEVKFNWYVDGVEVHNAKT |
| | | KPREEQYNSTYRVVSVLQVLHQDWLNGKEYKCKVSNKALPA |
| | | PIEKTISKAKGQPREPQVYTLPPSRDELTKNQVSLTCLVKGFYP |
| | | SDIAVEWESNGQPENNYKTTPPVLDSDGSFFLYSKLTVDKSRW |
| | | QQGNVFSCSVMHEALHNHYTQKSLSLSPG |
| hIgG1- | 430 | ASTKGPSVFPLAPSSKSTSGGTAALGCLVKDYFPEPVTVSWNS |
| LALA/DQ | | GALTSGVHTFPAVLQSSGLYSLSSVVTVPSSSLGTQTYICNVNH |
| | | KPSNTKVDKKVEPKSCDKTHTCPPCPAPEAAGGPSVFLFPPKP |
| | | KDTLMISRDPEVTCVVVDVSHEDPEVKFNWYVDGVEVHNAK |
| | | TKPREEQYNSTYRVVSVLQVLHQDWLNGKEYKCKVSNKALP |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVMHEALHNHYTQKSLSLSPG |
| hIgG1- | 431 | ASTKGPSVFPLAPSSKSTSGGTAALGCLVKDYFPEPVTVSWNS |
| LAGA/DQ | | GALTSGVHTFPAVLQSSGLYSLSSVVTVPSSSLGTQTYICNVNH |
| | | KPSNTKVDKKVEPKSCDKTHTCPPCPAPELAGAPSVFLFPPKP |
| | | KDTLMISRDPEVTCVVVDVSHEDPEVKFNWYVDGVEVHNAK |
| | | TKPREEQYNSTYRVVSVLQVLHQDWLNGKEYKCKVSNKALP |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVMHEALHNHYTQKSLSLSPG |
| hIgG1- | 432 | ASTKGPSVFPLAPSSKSTSGGTAALGCLVKDYFPEPVTVSWNS |
| LALAGA/ | | GALTSGVHTFPAVLQSSGLYSLSSVVTVPSSSLGTQTYICNVNH |
| DQ | | KPSNTKVDKKVEPKSCDKTHTCPPCPAPEAAGAPSVFLFPPKP |
| | | KDTLMISRDPEVTCVVVDVSHEDPEVKFNWYVDGVEVHNAK |
| | | TKPREEQYNSTYRVVSVLQVLHQDWLNGKEYKCKVSNKALP |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVMHEALHNHYTQKSLSLSPG |
| hIgG1- | 433 | ASTKGPSVFPLAPSSKSTSGGTAALGCLVKDYFPEPVTVSWNS |
| LALAPG/ | | GALTSGVHTFPAVLQSSGLYSLSSVVTVPSSSLGTQTYICNVNH |
| DQ | | KPSNTKVDKKVEPKSCDKTHTCPPCPAPEAAGGPSVFLFPPKP |
| | | KDTLMISRDPEVTCVVVDVSHEDPEVKFNWYVDGVEVHNAK |
| | | TKPREEQYNSTYRVVSVLQVLHQDWLNGKEYKCKVSNKALG |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVMHEALHNHYTQKSLSLSPG |
| hIgG1/DW | 434 | ASTKGPSVFPLAPSSKSTSGGTAALGCLVKDYFPEPVTVSWNS |
| | | GALTSGVHTFPAVLQSSGLYSLSSVVTVPSSSLGTQTYICNVNH |
| | | KPSNTKVDKKVEPKSCDKTHTCPPCPAPELLGGPSVFLFPPKPK |
| | | DTLMISRDPEVTCVVVDVSHEDPEVKFNWYVDGVEVHNAKT |
| | | KPREEQYNSTYRVVSVLWVLHQDWLNGKEYKCKVSNKALPA |
| | | PIEKTISKAKGQPREPQVYTLPPSRDELTKNQVSLTCLVKGFYP |
| | | SDIAVEWESNGQPENNYKTTPPVLDSDGSFFLYSKLTVDKSRW |
| | | QQGNVFSCSVMHEALHNHYTQKSLSLSPG |
| hIgG1- | 435 | ASTKGPSVFPLAPSSKSTSGGTAALGCLVKDYFPEPVTVSWNS |
| N297A/DW | | GALTSGVHTFPAVLQSSGLYSLSSVVTVPSSSLGTQTYICNVNH |
| | | KPSNTKVDKKVEPKSCDKTHTCPPCPAPELLGGPSVFLFPPKPK |
| | | DTLMISRDPEVTCVVVDVSHEDPEVKFNWYVDGVEVHNAKT |
| | | KPREEQYASTYRVVSVLWVLHQDWLNGKEYKCKVSNKALPA |
| | | PIEKTISKAKGQPREPQVYTLPPSRDELTKNQVSLTCLVKGFYP |
| | | SDIAVEWESNGQPENNYKTTPPVLDSDGSFFLYSKLTVDKSRW |
| | | QQGNVFSCSVMHEALHNHYTQKSLSLSPG |
| hIgG1- | 436 | ASTKGPSVFPLAPSSKSTSGGTAALGCLVKDYFPEPVTVSWNS |
| D265A/DW | | GALTSGVHTFPAVLQSSGLYSLSSVVTVPSSSLGTQTYICNVNH |
| | | KPSNTKVDKKVEPKSCDKTHTCPPCPAPELLGGPSVFLFPPKPK |
| | | DTLMISRDPEVTCVVVAVSHEDPEVKFNWYVDGVEVHNAKT |
| | | KPREEQYNSTYRVVSVLWVLHQDWLNGKEYKCKVSNKALPA |
| | | PIEKTISKAKGQPREPQVYTLPPSRDELTKNQVSLTCLVKGFYP |
| | | SDIAVEWESNGQPENNYKTTPPVLDSDGSFFLYSKLTVDKSRW |
| | | QQGNVFSCSVMHEALHNHYTQKSLSLSPG |
| hIgG1- | 437 | ASTKGPSVFPLAPSSKSTSGGTAALGCLVKDYFPEPVTVSWNS |
| LALA/DW | | GALTSGVHTFPAVLQSSGLYSLSSVVTVPSSSLGTQTYICNVNH |
| | | KPSNTKVDKKVEPKSCDKTHTCPPCPAPEAAGGPSVFLFPPKP |
| | | KDTLMISRDPEVTCVVVDVSHEDPEVKFNWYVDGVEVHNAK |
| | | TKPREEQYNSTYRVVSVLWVLHQDWLNGKEYKCKVSNKALP |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVMHEALHNHYTQKSLSLSPG |
| hIgG1- | 438 | ASTKGPSVFPLAPSSKSTSGGTAALGCLVKDYFPEPVTVSWNS |
| LAGA/DW | | GALTSGVHTFPAVLQSSGLYSLSSVVTVPSSSLGTQTYICNVNH |
| | | KPSNTKVDKKVEPKSCDKTHTCPPCPAPELAGAPSVFLFPPKP |
| | | KDTLMISRDPEVTCVVVDVSHEDPEVKFNWYVDGVEVHNAK |
| | | TKPREEQYNSTYRVVSVLWVLHQDWLNGKEYKCKVSNKALP |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVMHEALHNHYTQKSLSLSPG |
| hIgG1- | 439 | ASTKGPSVFPLAPSSKSTSGGTAALGCLVKDYFPEPVTVSWNS |
| LALAGA/ | | GALTSGVHTFPAVLQSSGLYSLSSVVTVPSSSLGTQTYICNVNH |
| DW | | KPSNTKVDKKVEPKSCDKTHTCPPCPAPEAAGAPSVFLFPPKP |
| | | KDTLMISRDPEVTCVVVDVSHEDPEVKFNWYVDGVEVHNAK |
| | | TKPREEQYNSTYRVVSVLWVLHQDWLNGKEYKCKVSNKALP |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVMHEALHNHYTQKSLSLSPG |
| hIgG1- | 440 | ASTKGPSVFPLAPSSKSTSGGTAALGCLVKDYFPEPVTVSWNS |
| LALAPG/ | | GALTSGVHTFPAVLQSSGLYSLSSVVTVPSSSLGTQTYICNVNH |
| DW | | KPSNTKVDKKVEPKSCDKTHTCPPCPAPEAAGGPSVFLFPPKP |
| | | KDTLMISRDPEVTCVVVDVSHEDPEVKFNWYVDGVEVHNAK |
| | | TKPREEQYNSTYRVVSVLWVLHQDWLNGKEYKCKVSNKALG |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVMHEALHNHYTQKSLSLSPG |
| hIgG1/YD | 441 | ASTKGPSVFPLAPSSKSTSGGTAALGCLVKDYFPEPVTVSWNS |
| | | GALTSGVHTFPAVLQSSGLYSLSSVVTVPSSSLGTQTYICNVNH |
| | | KPSNTKVDKKVEPKSCDKTHTCPPCPAPELLGGPSVFLFPPKPK |
| | | DTLYISRDPEVTCVVVDVSHEDPEVKFNWYVDGVEVHNAKT |
| | | KPREEQYNSTYRVVSVLTVLHQDWLNGKEYKCKVSNKALPA |
| | | PIEKTISKAKGQPREPQVYTLPPSRDELTKNQVSLTCLVKGFYP |
| | | SDIAVEWESNGQPENNYKTTPPVLDSDGSFFLYSKLTVDKSRW |
| | | QQGNVFSCSVMHEALHNHYTQKSLSLSPG |
| hIgG1- | 442 | ASTKGPSVFPLAPSSKSTSGGTAALGCLVKDYFPEPVTVSWNS |
| N297A/YD | | GALTSGVHTFPAVLQSSGLYSLSSVVTVPSSSLGTQTYICNVNH |
| | | KPSNTKVDKKVEPKSCDKTHTCPPCPAPELLGGPSVFLFPPKPK |
| | | DTLYISRDPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVYTLPPSRDELTKNQVSLTCLVKGFYP |
| | | SDIAVEWESNGQPENNYKTTPPVLDSDGSFFLYSKLTVDKSRW |
| | | QQGNVFSCSVMHEALHNHYTQKSLSLSPG |
| hIgG1- | 443 | ASTKGPSVFPLAPSSKSTSGGTAALGCLVKDYFPEPVTVSWNS |
| D265A/YD | | GALTSGVHTFPAVLQSSGLYSLSSVVTVPSSSLGTQTYICNVNH |
| | | KPSNTKVDKKVEPKSCDKTHTCPPCPAPELLGGPSVFLFPPKPK |
| | | DTLYISRDPEVTCVVVAVSHEDPEVKFNWYVDGVEVHNAKT |
| | | KPREEQYNSTYRVVSVLTVLHQDWLNGKEYKCKVSNKALPA |
| | | PIEKTISKAKGQPREPQVYTLPPSRDELTKNQVSLTCLVKGFYP |
| | | SDIAVEWESNGQPENNYKTTPPVLDSDGSFFLYSKLTVDKSRW |
| | | QQGNVFSCSVMHEALHNHYTQKSLSLSPG |
| hIgG1- | 444 | ASTKGPSVFPLAPSSKSTSGGTAALGCLVKDYFPEPVTVSWNS |
| LALA/YD | | GALTSGVHTFPAVLQSSGLYSLSSVVTVPSSSLGTQTYICNVNH |
| | | KPSNTKVDKKVEPKSCDKTHTCPPCPAPEAAGGPSVFLFPPKP |
| | | KDTLYISRDPEVTCVVVDVSHEDPEVKFNWYVDGVEVHNAK |
| | | TKPREEQYNSTYRVVSVLTVLHQDWLNGKEYKCKVSNKALP |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVMHEALHNHYTQKSLSLSPG |
| hIgG1- | 445 | ASTKGPSVFPLAPSSKSTSGGTAALGCLVKDYFPEPVTVSWNS |
| LAGA/YD | | GALTSGVHTFPAVLQSSGLYSLSSVVTVPSSSLGTQTYICNVNH |
| | | KPSNTKVDKKVEPKSCDKTHTCPPCPAPELAGAPSVFLFPPKP |
| | | KDTLYISRDPEVTCVVVDVSHEDPEVKFNWYVDGVEVHNAK |
| | | TKPREEQYNSTYRVVSVLTVLHQDWLNGKEYKCKVSNKALP |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVMHEALHNHYTQKSLSLSPG |
| hIgG1- | 446 | ASTKGPSVFPLAPSSKSTSGGTAALGCLVKDYFPEPVTVSWNS |
| LALAGA/ | | GALTSGVHTFPAVLQSSGLYSLSSVVTVPSSSLGTQTYICNVNH |
| YD | | KPSNTKVDKKVEPKSCDKTHTCPPCPAPEAAGAPSVFLFPPKP |
| | | KDTLYISRDPEVTCVVVDVSHEDPEVKFNWYVDGVEVHNAK |
| | | TKPREEQYNSTYRVVSVLTVLHQDWLNGKEYKCKVSNKALP |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVMHEALHNHYTQKSLSLSPG |
| hIgG1- | 447 | ASTKGPSVFPLAPSSKSTSGGTAALGCLVKDYFPEPVTVSWNS |
| LALAPG/ | | GALTSGVHTFPAVLQSSGLYSLSSVVTVPSSSLGTQTYICNVNH |
| YD | | KPSNTKVDKKVEPKSCDKTHTCPPCPAPEAAGGPSVFLFPPKP |
| | | KDTLYISRDPEVTCVVVDVSHEDPEVKFNWYVDGVEVHNAK |
| | | TKPREEQYNSTYRVVSVLTVLHQDWLNGKEYKCKVSNKALG |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVMHEALHNHYTQKSLSLSPG |
| hIgG1/QVV | 448 | ASTKGPSVFPLAPSSKSTSGGTAALGCLVKDYFPEPVTVSWNS |
| | | GALTSGVHTFPAVLQSSGLYSLSSVVTVPSSSLGTQTYICNVNH |
| | | KPSNTKVDKKVEPKSCDKTHTCPPCPAPELLGGPSVFLFPPKPK |
| | | DTLMISRTPEVTCVVVDVSHEDPEVKFNWYVDGVEVHNAKT |
| | | KPREEQYNSTYRVVSVLQVLHVDWLNGKEYKCKVSNKALPA |
| | | PIEKTISKAKGQPREPQVYTLPPSRDELTKNQVSLTCLVKGFYP |
| | | SDIVVEWESNGQPENNYKTTPPVLDSDGSFFLYSKLTVDKSRW |
| | | QQGNVFSCSVMHEALHNHYTQKSLSLSPG |
| hIgG1- | 449 | ASTKGPSVFPLAPSSKSTSGGTAALGCLVKDYFPEPVTVSWNS |
| N297A/QVV | | GALTSGVHTFPAVLQSSGLYSLSSVVTVPSSSLGTQTYICNVNH |
| | | KPSNTKVDKKVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLQVLHVDWLNGKEYKCKVSNKALPA |
| | | PIEKTISKAKGQPREPQVYTLPPSRDELTKNQVSLTCLVKGFYP |
| | | SDIVVEWESNGQPENNYKTTPPVLDSDGSFFLYSKLTVDKSRW |
| | | QQGNVFSCSVMHEALHNHYTQKSLSLSPG |
| hIgG1- | 450 | ASTKGPSVFPLAPSSKSTSGGTAALGCLVKDYFPEPVTVSWNS |
| D265A/QVV | | GALTSGVHTFPAVLQSSGLYSLSSVVTVPSSSLGTQTYICNVNH |
| | | KPSNTKVDKKVEPKSCDKTHTCPPCPAPELLGGPSVFLFPPKPK |
| | | DTLMISRTPEVTCVVVAVSHEDPEVKFNWYVDGVEVHNAKT |
| | | KPREEQYNSTYRVVSVLQVLHVDWLNGKEYKCKVSNKALPA |
| | | PIEKTISKAKGQPREPQVYTLPPSRDELTKNQVSLTCLVKGFYP |
| | | SDIVVEWESNGQPENNYKTTPPVLDSDGSFFLYSKLTVDKSRW |
| | | QQGNVFSCSVMHEALHNHYTQKSLSLSPG |
| hIgG1- | 451 | ASTKGPSVFPLAPSSKSTSGGTAALGCLVKDYFPEPVTVSWNS |
| LALA/QVV | | GALTSGVHTFPAVLQSSGLYSLSSVVTVPSSSLGTQTYICNVNH |
| | | KPSNTKVDKKVEPKSCDKTHTCPPCPAPEAAGGPSVFLFPPKP |
| | | KDTLMISRTPEVTCVVVDVSHEDPEVKFNWYVDGVEVHNAK |
| | | TKPREEQYNSTYRVVSVLQVLHVDWLNGKEYKCKVSNKALP |
| | | APIEKTISKAKGQPREPQVYTLPPSRDELTKNQVSLTCLVKGFY |
| | | PSDIVVEWESNGQPENNYKTTPPVLDSDGSFFLYSKLTVDKSR |
| | | WQQGNVFSCSVMHEALHNHYTQKSLSLSPG |
| hIgG1- | 452 | ASTKGPSVFPLAPSSKSTSGGTAALGCLVKDYFPEPVTVSWNS |
| LAGA/QVV | | GALTSGVHTFPAVLQSSGLYSLSSVVTVPSSSLGTQTYICNVNH |
| | | KPSNTKVDKKVEPKSCDKTHTCPPCPAPELAGAPSVFLFPPKP |
| | | KDTLMISRTPEVTCVVVDVSHEDPEVKFNWYVDGVEVHNAK |
| | | TKPREEQYNSTYRVVSVLQVLHVDWLNGKEYKCKVSNKALP |
| | | APIEKTISKAKGQPREPQVYTLPPSRDELTKNQVSLTCLVKGFY |
| | | PSDIVVEWESNGQPENNYKTTPPVLDSDGSFFLYSKLTVDKSR |
| | | WQQGNVFSCSVMHEALHNHYTQKSLSLSPG |
| hIgG1- | 453 | ASTKGPSVFPLAPSSKSTSGGTAALGCLVKDYFPEPVTVSWNS |
| LALAGA/ | | GALTSGVHTFPAVLQSSGLYSLSSVVTVPSSSLGTQTYICNVNH |
| QVV | | KPSNTKVDKKVEPKSCDKTHTCPPCPAPEAAGAPSVFLFPPKP |
| | | KDTLMISRTPEVTCVVVDVSHEDPEVKFNWYVDGVEVHNAK |
| | | TKPREEQYNSTYRVVSVLQVLHVDWLNGKEYKCKVSNKALP |
| | | APIEKTISKAKGQPREPQVYTLPPSRDELTKNQVSLTCLVKGFY |
| | | PSDIVVEWESNGQPENNYKTTPPVLDSDGSFFLYSKLTVDKSR |
| | | WQQGNVFSCSVMHEALHNHYTQKSLSLSPG |
| hIgG1- | 454 | ASTKGPSVFPLAPSSKSTSGGTAALGCLVKDYFPEPVTVSWNS |
| LALAPG/ | | GALTSGVHTFPAVLQSSGLYSLSSVVTVPSSSLGTQTYICNVNH |
| QVV | | KPSNTKVDKKVEPKSCDKTHTCPPCPAPEAAGGPSVFLFPPKP |
| | | KDTLMISRTPEVTCVVVDVSHEDPEVKFNWYVDGVEVHNAK |
| | | TKPREEQYNSTYRVVSVLQVLHVDWLNGKEYKCKVSNKALG |
| | | APIEKTISKAKGQPREPQVYTLPPSRDELTKNQVSLTCLVKGFY |
| | | PSDIVVEWESNGQPENNYKTTPPVLDSDGSFFLYSKLTVDKSR |
| | | WQQGNVFSCSVMHEALHNHYTQKSLSLSPG |
| hIgG1/ | 455 | ASTKGPSVFPLAPSSKSTSGGTAALGCLVKDYFPEPVTVSWNS |
| DDRVV | | GALTSGVHTFPAVLQSSGLYSLSSVVTVPSSSLGTQTYICNVNH |
| | | KPSNTKVDKKVEPKSCDKTHTCPPCPAPELLGGPSVFLFPPKPK |
| | | DTLMISRDPEVTCVVVDVSHEDPEVKFNWYVDGVEVDNAKT |
| | | KPREEQYNSTYRVVSVLRVLHVDWLNGKEYKCKVSNKALPA |
| | | PIEKTISKAKGQPREPQVYTLPPSRDELTKNQVSLTCLVKGFYP |
| | | SDIVVEWESNGQPENNYKTTPPVLDSDGSFFLYSKLTVDKSRW |
| | | QQGNVFSCSVMHEALHNHYTQKSLSLSPG |
| hIgG1- | 456 | ASTKGPSVFPLAPSSKSTSGGTAALGCLVKDYFPEPVTVSWNS |
| N297A/ | | GALTSGVHTFPAVLQSSGLYSLSSVVTVPSSSLGTQTYICNVNH |
| DDRVV | | KPSNTKVDKKVEPKSCDKTHTCPPCPAPELLGGPSVFLFPPKPK |
| | | DTLMISRDPEVTCVVVDVSHEDPEVKFNWYVDGVEVDNAKT |
| | | KPREEQYASTYRVVSVLRVLHVDWLNGKEYKCKVSNKALPA |
| | | PIEKTISKAKGQPREPQVYTLPPSRDELTKNQVSLTCLVKGFYP |
| | | SDIVVEWESNGQPENNYKTTPPVLDSDGSFFLYSKLTVDKSRW |
| | | QQGNVFSCSVMHEALHNHYTQKSLSLSPG |
| hIgG1- | 457 | ASTKGPSVFPLAPSSKSTSGGTAALGCLVKDYFPEPVTVSWNS |
| D265A/ | | GALTSGVHTFPAVLQSSGLYSLSSVVTVPSSSLGTQTYICNVNH |
| DDRVV | | KPSNTKVDKKVEPKSCDKTHTCPPCPAPELLGGPSVFLFPPKPK |
| | | DTLMISRDPEVTCVVVAVSHEDPEVKFNWYVDGVEVDNAKT |
| | | KPREEQYNSTYRVVSVLRVLHVDWLNGKEYKCKVSNKALPA |
| | | PIEKTISKAKGQPREPQVYTLPPSRDELTKNQVSLTCLVKGFYP |
| | | SDIVVEWESNGQPENNYKTTPPVLDSDGSFFLYSKLTVDKSRW |
| | | QQGNVFSCSVMHEALHNHYTQKSLSLSPG |
| hIgG1- | 458 | ASTKGPSVFPLAPSSKSTSGGTAALGCLVKDYFPEPVTVSWNS |
| LALA/ | | GALTSGVHTFPAVLQSSGLYSLSSVVTVPSSSLGTQTYICNVNH |
| DDRVV | | KPSNTKVDKKVEPKSCDKTHTCPPCPAPEAAGGPSVFLFPPKP |
| | | KDTLMISRDPEVTCVVVDVSHEDPEVKFNWYVDGVEVDNAK |
| | | TKPREEQYNSTYRVVSVLRVLHVDWLNGKEYKCKVSNKALP |
| | | APIEKTISKAKGQPREPQVYTLPPSRDELTKNQVSLTCLVKGFY |
| | | PSDIVVEWESNGQPENNYKTTPPVLDSDGSFFLYSKLTVDKSR |
| | | WQQGNVFSCSVMHEALHNHYTQKSLSLSPG |
| hIgG1- | 459 | ASTKGPSVFPLAPSSKSTSGGTAALGCLVKDYFPEPVTVSWNS |
| LAGA/ | | GALTSGVHTFPAVLQSSGLYSLSSVVTVPSSSLGTQTYICNVNH |
| DDRVV | | KPSNTKVDKKVEPKSCDKTHTCPPCPAPELAGAPSVFLFPPKP |
| | | KDTLMISRDPEVTCVVVDVSHEDPEVKFNWYVDGVEVDNAK |
| | | TKPREEQYNSTYRVVSVLRVLHVDWLNGKEYKCKVSNKALP |
| | | APIEKTISKAKGQPREPQVYTLPPSRDELTKNQVSLTCLVKGFY |
| | | PSDIVVEWESNGQPENNYKTTPPVLDSDGSFFLYSKLTVDKSR |
| | | WQQGNVFSCSVMHEALHNHYTQKSLSLSPG |
| hIgG1- | 460 | ASTKGPSVFPLAPSSKSTSGGTAALGCLVKDYFPEPVTVSWNS |
| LALAGA/ | | GALTSGVHTFPAVLQSSGLYSLSSVVTVPSSSLGTQTYICNVNH |
| DDRVV | | KPSNTKVDKKVEPKSCDKTHTCPPCPAPEAAGAPSVFLFPPKP |
| | | KDTLMISRDPEVTCVVVDVSHEDPEVKFNWYVDGVEVDNAK |
| | | TKPREEQYNSTYRVVSVLRVLHVDWLNGKEYKCKVSNKALP |
| | | APIEKTISKAKGQPREPQVYTLPPSRDELTKNQVSLTCLVKGFY |
| | | PSDIVVEWESNGQPENNYKTTPPVLDSDGSFFLYSKLTVDKSR |
| | | WQQGNVFSCSVMHEALHNHYTQKSLSLSPG |
| hIgG1- | 461 | ASTKGPSVFPLAPSSKSTSGGTAALGCLVKDYFPEPVTVSWNS |
| LALAPG/ | | GALTSGVHTFPAVLQSSGLYSLSSVVTVPSSSLGTQTYICNVNH |
| DDRVV | | KPSNTKVDKKVEPKSCDKTHTCPPCPAPEAAGGPSVFLFPPKP |
| | | KDTLMISRDPEVTCVVVDVSHEDPEVKFNWYVDGVEVDNAK |
| | | TKPREEQYNSTYRVVSVLRVLHVDWLNGKEYKCKVSNKALG |
| | | APIEKTISKAKGQPREPQVYTLPPSRDELTKNQVSLTCLVKGFY |
| | | PSDIVVEWESNGQPENNYKTTPPVLDSDGSFFLYSKLTVDKSR |
| | | WQQGNVFSCSVMHEALHNHYTQKSLSLSPG |
| hIgG1- | 462 | ASTKGPSVFPLAPSSKSTSGGTAALGCLVKDYFPEPVTVSWNS |
| Q311R/ | | GALTSGVHTFPAVLQSSGLYSLSSVVTVPSSSLGTQTYICNVNH |
| M428L | | KPSNTKVDKKVEPKSCDKTHTCPPCPAPELLGGPSVFLFPPKPK |
| | | DTLMISRTPEVTCVVVDVSHEDPEVKFNWYVDGVEVHNAKT |
| | | KPREEQYNSTYRVVSVLTVLHRDWLNGKEYKCKVSNKALPA |
| | | PIEKTISKAKGQPREPQVYTLPPSRDELTKNQVSLTCLVKGFYP |
| | | SDIAVEWESNGQPENNYKTTPPVLDSDGSFFLYSKLTVDKSRW |
| | | QQGNVFSCSVLHEALHNHYTQKSLSLSPG |
| hIgG4- | 463 | ASTKGPSVFPLAPCSRSTSESTAALGCLVKDYFPEPVTVSWNS |
| Q311R/ | | GALTSGVHTFPAVLQSSGLYSLSSVVTVPSSSLGTKTYTCNVD |
| M428L | | HKPSNTKVDKRVESKYGPPCPSCPAPEFLGGPSVFLFPPKPKDT |
| | | LMISRTPEVTCVVVDVSQEDPEVQFNWYVDGVEVHNAKTKP |
| | | REEQFNSTYRVVSVLTVLHRDWLNGKEYKCKVSNKGLPSSIE |
| | | KTISKAKGQPREPQVYTLPPSQEEMTKNQVSLTCLVKGFYPSD |
| | | IAVEWESNGQPENNYKTTPPVLDSDGSFFLYSRLTVDKSRWQE |
| | | GNVFSCSVLHEALHNHYTQKSLSLSLGK |
| IgG4- | 464 | ASTKGPSVFPLAPCSRSTSESTAALGCLVKDYFPEPVTVSWNS |
| SP/Q311R/ | | GALTSGVHTFPAVLQSSGLYSLSSVVTVPSSSLGTKTYTCNVD |
| M428L | | HKPSNTKVDKRVESKYGPPCPPCPAPEFLGGPSVFLFPPKPKDT |
| | | LMISRTPEVTCVVVDVSQEDPEVQFNWYVDGVEVHNAKTKP |
| | | REEQFNSTYRVVSVLTVLHRDWLNGKEYKCKVSNKGLPSSIE |
| | | KTISKAKGQPREPQVYTLPPSQEEMTKNQVSLTCLVKGFYPSD |
| | | IAVEWESNGQPENNYKTTPPVLDSDGSFFLYSRLTVDKSRWQE |
| | | GNVFSCSVLHEALHNHYTQKSLSLSLGK |
| IgG4- | 465 | ASTKGPSVFPLAPCSRSTSESTAALGCLVKDYFPEPVTVSWNS |
| SPLE/ | | GALTSGVHTFPAVLQSSGLYSLSSVVTVPSSSLGTKTYTCNVD |
| Q311R/ | | HKPSNTKVDKRVESKYGPPCPPCPAPEELGGPSVFLFPPKPKDT |
| M428L | | LMISRTPEVTCVVVDVSQEDPEVQFNWYVDGVEVHNAKTKP |
| | | REEQFNSTYRVVSVLTVLHRDWLNGKEYKCKVSNKGLPSSIE |
| | | KTISKAKGQPREPQVYTLPPSQEEMTKNQVSLTCLVKGFYPSD |
| | | IAVEWESNGQPENNYKTTPPVLDSDGSFFLYSRLTVDKSRWQE |
| | | GNVFSCSVLHEALHNHYTQKSLSLSLGK |
| IgG2- | 466 | ASTKGPSVFPLAPCSRSTSESTAALGCLVKDYFPEPVTVSWNS |
| Q311R/ | | GALTSGVHTFPAVLQSSGLYSLSSVVTVPSSNFGTQTYTCNVD |
| M428L | | HKPSNTKVDKTVERKCCVECPPCPAPPVAGPSVFLFPPKPKDT |
| | | LMISRTPEVTCVVVDVSHEDPEVQFNWYVDGVEVHNAKTKP |
| | | REEQFNSTFRVVSVLTVVHRDWLNGKEYKCKVSNKGLPAPIE |
| | | KTISKTKGQPREPQVYTLPPSREEMTKNQVSLTCLVKGFYPSDI |
| | | AVEWESNGQPENNYKTTPPMLDSDGSFFLYSKLTVDKSRWQQ |
| | | GNVFSCSVLHEALHNHYTQKSLSLSP |
| hIgG1- | 467 | ASTKGPSVFPLAPSSKSTSGGTAALGCLVKDYFPEPVTVSWNS |
| N297A/ | | GALTSGVHTFPAVLQSSGLYSLSSVVTVPSSSLGTQTYICNVNH |
| Q311R/ | | KPSNTKVDKKVEPKSCDKTHTCPPCPAPELLGGPSVFLFPPKPK |
| M428L | | DTLMISRTPEVTCVVVDVSHEDPEVKFNWYVDGVEVHNAKT |
| | | KPREEQYASTYRVVSVLTVLHRDWLNGKEYKCKVSNKALPA |
| | | PIEKTISKAKGQPREPQVYTLPPSRDELTKNQVSLTCLVKGFYP |
| | | SDIAVEWESNGQPENNYKTTPPVLDSDGSFFLYSKLTVDKSRW |
| | | QQGNVFSCSVLHEALHNHYTQKSLSLSPG |
| hIgG1- | 468 | ASTKGPSVFPLAPSSKSTSGGTAALGCLVKDYFPEPVTVSWNS |
| D265A/ | | GALTSGVHTFPAVLQSSGLYSLSSVVTVPSSSLGTQTYICNVNH |
| Q311R/ | | KPSNTKVDKKVEPKSCDKTHTCPPCPAPELLGGPSVFLFPPKPK |
| M428L | | DTLMISRTPEVTCVVVAVSHEDPEVKFNWYVDGVEVHNAKT |
| | | KPREEQYNSTYRVVSVLTVLHRDWLNGKEYKCKVSNKALPA |
| | | PIEKTISKAKGQPREPQVYTLPPSRDELTKNQVSLTCLVKGFYP |
| | | SDIAVEWESNGQPENNYKTTPPVLDSDGSFFLYSKLTVDKSRW |
| | | QQGNVFSCSVLHEALHNHYTQKSLSLSPG |
| hIgG1- | 469 | ASTKGPSVFPLAPSSKSTSGGTAALGCLVKDYFPEPVTVSWNS |
| LALA/ | | GALTSGVHTFPAVLQSSGLYSLSSVVTVPSSSLGTQTYICNVNH |
| Q311R/ | | KPSNTKVDKKVEPKSCDKTHTCPPCPAPEAAGGPSVFLFPPKP |
| M428L | | KDTLMISRTPEVTCVVVDVSHEDPEVKFNWYVDGVEVHNAK |
| | | TKPREEQYNSTYRVVSVLTVLHRDWLNGKEYKCKVSNKALP |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVLHEALHNHYTQKSLSLSPG |
| hIgG1- | 470 | ASTKGPSVFPLAPSSKSTSGGTAALGCLVKDYFPEPVTVSWNS |
| LAGA/ | | GALTSGVHTFPAVLQSSGLYSLSSVVTVPSSSLGTQTYICNVNH |
| Q311R/ | | KPSNTKVDKKVEPKSCDKTHTCPPCPAPELAGAPSVFLFPPKP |
| M428L | | KDTLMISRTPEVTCVVVDVSHEDPEVKFNWYVDGVEVHNAK |
| | | TKPREEQYNSTYRVVSVLTVLHRDWLNGKEYKCKVSNKALP |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVLHEALHNHYTQKSLSLSPG |
| hIgG1- | 471 | ASTKGPSVFPLAPSSKSTSGGTAALGCLVKDYFPEPVTVSWNS |
| LALAGA/ | | GALTSGVHTFPAVLQSSGLYSLSSVVTVPSSSLGTQTYICNVNH |
| Q311R/ | | KPSNTKVDKKVEPKSCDKTHTCPPCPAPEAAGAPSVFLFPPKP |
| M428L | | KDTLMISRTPEVTCVVVDVSHEDPEVKFNWYVDGVEVHNAK |
| | | TKPREEQYNSTYRVVSVLTVLHRDWLNGKEYKCKVSNKALP |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVLHEALHNHYTQKSLSLSPG |
| hIgG1- | 472 | ASTKGPSVFPLAPSSKSTSGGTAALGCLVKDYFPEPVTVSWNS |
| LALAPG/ | | GALTSGVHTFPAVLQSSGLYSLSSVVTVPSSSLGTQTYICNVNH |
| Q311R/ | | KPSNTKVDKKVEPKSCDKTHTCPPCPAPEAAGGPSVFLFPPKP |
| M428L | | KDTLMISRTPEVTCVVVDVSHEDPEVKFNWYVDGVEVHNAK |
| | | TKPREEQYNSTYRVVSVLTVLHRDWLNGKEYKCKVSNKALG |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSR |
| | | WQQGNVFSCSVLHEALHNHYTQKSLSLSPG |

In some embodiments, the TL1A binding protein comprises a Fc comprising one or more modifications in SEQ ID NO: 361. In some embodiments, the TL1A binding protein comprises a Fc comprising one or more modifications in SEQ ID NO: 362. In some embodiments, the TL1A binding protein comprises a Fc comprising one or more modifications in SEQ ID NO: 363. In some embodiments, the Fc comprises an amino acid sequence having at least 80% sequence identity with the amino acid sequence according to any one of SEQ ID NOs: 361-363. In some embodiments, the Fc comprises an amino acid sequence having at least 85% sequence identity with the amino acid sequence according to any one of SEQ ID NOs: 361-363. In some embodiments, the Fc comprises an amino acid sequence having at least 9000 sequence identity with the amino acid sequence according to any one of SEQ ID NOs: 361-363. In some embodiments, the Fc comprises an amino acid sequence having at least 95% sequence identity with the amino acid sequence according to any one of SEQ ID NOs: 361-363. In some embodiments, the Fc comprises an amino acid sequence having at least 96% sequence identity with the amino acid sequence according to any one of SEQ ID NOs: 361-363. In some embodiments, the Fc comprises an amino acid sequence having at least 97% sequence identity with the amino acid sequence according to any one of SEQ ID NOs: 361-363. In some embodiments, the Fc comprises an amino acid sequence having at least 98% sequence identity with the amino acid sequence according to any one of SEQ ID NOs: 361-363. In some embodiments, the Fc comprises an amino acid sequence having at least 99% sequence identity with the amino acid sequence according to any one of SEQ ID NOs: 361-363. In some embodiments, the Fc comprises the amino acid sequence according to any one of SEQ ID NOs: 361-363.

In some embodiments, one or more modifications in the modified Fc is selected from the group consisting of S298A, E333A, K334A, K326A, F243L, R292P, Y300L, V3051, P396L, F243L, R292P, Y300L, L235V, P396L, F243L, S239D, I332E, A330L, S267E, L328F, D265S, S239E, K326A, A327H, G237F, K326E, G236A, D270L, H268D, S324T, L234F, N325L, V266L, and S267D. In some embodiments, one or more modifications in the modified Fc is selected from the group consisting of S228P, M252Y, S254T, T256E, T256D, T250Q, H285D, T307A, T307Q, T307R, T307W, L309D, Q411H, Q311V, A378V, E380A, M428L, N434A, N434S, N297A, D265A, L234A, L235A, and N434W.

In some embodiments, the modified Fc comprises a specific combination of amino acid substitutions selected from the group consisting of L234A/L235A; V234A/G237A; L235A/G237A/E318A; S228P/L236E; H268Q/V309L/A330S/A331S; C220S/C226S/C229S/P238S; C226S/C229S/E3233P/L235V/L235A; L234F/L235E/P331S; C226S/P230S; L234A/G237A; L234A/L235A/G237A; Q311R/M428L; and L234A/L235A/P329G.

In some embodiments, the modified Fc comprises a specific combination of amino acid substitutions selected from the group consisting of M428L/N434S (LS); M252Y/S254T/T256E (YTE); T250Q/M428L; T307A/E380A/N434A; T256D/T307Q (DQ); T256D/T307W (DW); M252Y/T256D (YD); T307Q/Q311V/A378V (QVV); T256D/H285D/T307R/Q311V/A378V (DDRVV); L309D/Q311H/N434S (DHS); S228P/L235E (SPLE); L234A/L235A (LALA); M428L/N434A (LA); L234A/G237A (LAGA); L234A/L235A/G237A (LALAGA); L234A/L235A/P329G (LALAPG); N297A/YTE; D265A/YTE; LALA/YTE; LAGA/YTE; LALAGA/YTE; LALAPG/YTE; N297A/LS; D265A/LS; LALA/LS; LAGA/LS; LALAGA/LS; LALAPG/LS; N297A/DHS; D265A/DHS; LALA/DHS; LAGA/DHS; LALAGA/DHS; LALAPG/DHS; SP/YTE; SPLE/YTE; SP/LS; SPLE/LS; SP/DHS; SPLE/DHS; N297A/LA; D265A/LA; LALA/LA; LAGA/LA; LALAGA/LA; LALAPG/LA; N297A/N434A; D265A/N434A; LALA/N434A; LAGA/N434A; LALAGA/N434A; LALAPG/N434A; N297A/N434W; D265A/N434W; LALA/N434W; LAGA/N434W; LALAGA/N434W; LALAPG/N434W; N297A/DQ; D265A/DQ; LALA/DQ; LAGA/DQ; LALAGA/DQ; LALAPG/DQ; N297A/DW; D265A/DW; LALA/DW; LAGA/DW; LALAGA/DW; LALAPG/DW; N297A/YD; D265A/YD; LALA/YD; LAGA/YD; LALAGA/YD; LALAPG/YD; N297A/QVV; D265A/QVV; LALA/QVV; LAGA/QVV, LALAGA/QVV; LALAPG/QVV; N297A/DDRVV; D265A/DDRVV; LALA/DDRVV; LAGA/DDRVV; LALAGA/DDRVV; LALAPG/DDRVV; SP/Q311R/M428L; SPLE/Q311R/M428L; N297A/Q311R/M428L; D265A/Q311R/M428L; LALA/Q311R/M428L; LAGA/Q311R/M428L; LALAGA/Q311R/M428L; and LALAPG/Q311R/M428L. In some embodiments, the modified Fc comprises a specific combination of amino acid substitutions selected from the group consisting of M428L/N434S (LS) and M252Y/S254T/T256E (YTE). In some embodiments, the modified Fc comprises M428L/N434S (LS) (e.g., SEQ ID NO: 379, SEQ ID NO: 396, SEQ ID NO: 403) modifications. In some embodiments, the modified Fc comprises M252Y/S254T/T256E (YTE) (e.g., SEQ ID NO: 372, SEQ ID NO: 393, SEQ ID NO: 402) modifications.

In some embodiments, the TL1A binding proteins described herein include modifications to improve its ability to mediate effector function. Such modifications are known in the art and include afucosylation, or engineering of the affinity of the Fc towards an activating receptor, mainly FCGR3a for antibody-dependent cellular cytotoxicity (ADCC), and towards C1q for complement-dependent cytotoxicity (CDC).

In some aspects, an antibody provided herein comprises a Fc domain (e.g., IgG1) with reduced fucose content at position Asn 297 (EU numbering) compared to a naturally occurring Fc domain. Such Fc domains are known to have improved ADCC. In some aspects, such antibodies do not comprise any fucose at position Asn 297.

In some embodiments, the TL1A binding proteins described herein comprise an Fc region with one or more amino acid substitutions which improve ADCC, such as a substitution at one or more of positions 298, 333, and 334 of the Fc region. In some embodiments, an antibody provided herein comprises an Fc region with one or more amino acid substitutions at positions 239, 332, and 330.

In some embodiments, the Fc comprises an amino acid sequence having at least 80% sequence identity with the amino acid sequence according to any one of SEQ ID NOs: 364-472. In some embodiments, the Fc comprises an amino acid sequence having at least 85% sequence identity with the amino acid sequence according to any one of SEQ ID NOs: 364-472. In some embodiments, the Fc comprises an amino acid sequence having at least 90% sequence identity with the amino acid sequence according to any one of SEQ ID NOs: 364-472. In some embodiments, the Fc comprises an amino acid sequence having at least 95% sequence identity with the amino acid sequence according to any one of SEQ ID NOs: 364-472. In some embodiments, the Fc comprises an amino acid sequence having at least 96% sequence identity with the amino acid sequence according to any one of SEQ ID NOs: 364-472. In some embodiments, the Fc comprises an amino acid sequence having at least 97% sequence identity with the amino acid sequence according to any one of SEQ ID NOs: 364-472. In some embodiments, the Fc comprises an amino acid sequence having at least 98% sequence identity with the amino acid sequence according to any one of SEQ ID NOs: 364-472. In some embodiments, the Fc comprises an amino acid sequence having at least 99% sequence identity with the amino acid sequence according to any one of SEQ ID NOs: 364-472. In some embodiments, the Fc comprises the amino acid sequence according to any one of SEQ ID NOs: 364-472.

In some embodiments, the Fc of the TL1A binding protein comprises an amino acid sequence according to any one of SEQ ID NOs: 364-472, with one or more amino acid modifications (e.g., substitution, addition, deletion, e.g., relative to those set forth in any one of SEQ ID NOs: 364-472). In some embodiments, the one or more amino acid modifications comprises a deletion of a C-terminal lysine. In some embodiments, the one or more amino acid modifications comprises addition of a C-terminal lysine. To give but one example, the Fc of a TL1A binding protein provided herein can comprise an amino acid sequence according to SEQ ID NO: 376, but with a C-terminal lysine added such that the amino acid sequence ends with “K” rather than “G”.

In some embodiments, any of SEQ ID NOs: 346-472 that comprises a C-terminal lysine can have that C-terminal lysine deleted or substituted. In some embodiments, any of SEQ ID NOs: 346-472 that does not comprise a C-terminal lysine can have a C-terminal lysine added. In certain embodiments, any one of SEQ ID NOs: 346-472 that does not, itself, comprise a C-terminal lysine may also have its C-terminal amino acid substituted to a lysine (e.g., from another amino acid).

In some embodiments, the TL1A binding proteins described herein comprise an Fc region with at least one galactose residue in the oligosaccharide attached to the Fc region. Such antibody variants may have improved CDC function.

In some embodiments, the TL1A binding proteins described herein comprise one or more alterations that improve or diminish C1q binding and/or CDC.

In certain embodiments, the Fc region comprises one or more amino acid substitutions, wherein the one or more substitutions result in an increase in one or more of antibody half-life, ADCC activity, ADCP activity, or CDC activity compared with the Fc without the one or more substitutions. In certain embodiments, the one or more amino acid substitutions results in increased antibody half-life at pH 6.0 compared to an antibody comprising a wild-type Fc region. In certain embodiments, the antibody has an increased half-life that is about 10,000-fold, 1,000-fold, 500-fold, 100-fold, 50-fold, 20-fold, 10-fold, 9-fold, 8-fold, 7-fold, 6-fold, 5-fold, 4.5-fold, 4-fold, 3.5-fold, 3-fold, 2.5-fold, 2-fold, 1.95-fold, 1.9-fold, 1.85-fold, 1.8-fold, 1.75-fold, 1.7-fold, 1.65-fold, 1.6-fold, 1.55-fold, 1.50-fold, 1.45-fold, 1.4-fold, 1.35-fold, 1.3-fold, 1.25-fold, 1.2-fold, 1.15-fold, 1.1-fold, or 1.05-fold longer compared to an antibody comprising a wild-type Fc region.

In certain embodiments, the Fc region comprises one or more amino acid substitutions, wherein the one or more substitutions result in a decrease in one or more of ADCC activity, ADCP activity, or CDC activity compared with the Fc without the one or more substitutions.

In certain embodiments, the Fc region binds an Fcγ Receptor selected from the group consisting of FcγRI, FcγRIIa, FcγRIIb, FcγRIIc, FcγRIIIa, and FcγRIIIb. In certain embodiments, the Fc region binds an Fcγ Receptor with higher affinity at pH 6.0 compared to an antibody comprising a wild-type Fc region.

In some embodiments, the TL1A binding proteins described herein comprise an extended half-life (i.e., serum half-life). In some embodiments, the TL1A binding proteins described herein comprise a half-life of at least about 14, 28, 42, 56, 70, 84, 96, or more than 96 weeks. In some embodiments, the TL1A binding proteins described herein comprise a half-life in a range of about 14 days to about 96 days, about 14 days to about 84 days, about 14 days to about 70 days, about 14 days to about 56 days, about 14 days to about 42 days, about 14 days to about 28 days, of about 28 days to about 96 days, about 28 days to about 84 days, about 28 days to about 70 days, about 28 days to about 56 days, about 28 days to about 42 days, of about 42 days to about 96 days, about 42 days to about 84 days, about 42 days to about 70 days, or about 42 days to about 56 days. In some embodiments, the TL1A binding proteins described herein comprise a half-life in a range of about 42 days to about 56 days. In some embodiments, the TL1A binding proteins described herein comprise a half-life of at least about 50 days. In some embodiments, the TL1A binding proteins described herein comprise a half-life of about 50 days. Methods of measuring half-life are known in the art. In some embodiments, the half-life is measured in a non-human primate. In some embodiments, the half-life is measured in a human. In some embodiments, the half-life is measured following intravenous administration. In some embodiments, the half-life is measured following subcutaneous administration.

In some embodiments, the TL1A binding proteins described herein have a half-life that is at least 20% longer than a comparator antibody. In some embodiments, the comparator antibody comprises the same complementarity determining regions and variable regions but different Fc regions. In some embodiments, the half-life of the TL1A binding proteins described herein is at least 30%, at least 40%, at least 50%, at least 60%, at least 70%, at least 80%, or at least 90% longer than the half-life of the comparator antibody. In some embodiments, the half-life of the TL1A binding proteins described herein is longer than the half-life of the comparator antibody by at least 2 fold, at least 3 fold, at least 4 fold, at least 5 fold, at least 6 fold, at least 7 fold, at least 8 fold, at least 9 fold, or at least 10 fold.

### Methods of Treatment

Described herein, in certain embodiments, are methods of treating an inflammatory bowel disease in a patient in need thereof, the method comprising subcutaneously or intravenously administering to the patient an effective amount of an TL1A binding protein comprising a modified Fc region. Described herein, in certain embodiments, are methods of treating an inflammatory bowel disease in a patient in need thereof, the method comprising subcutaneously or intravenously administering to the patient an effective amount of an TL1A binding protein comprising a) a heavy chain variable region (VH) comprising (i) a CDR1 having an amino acid sequence according to any one of SEQ ID NOs: 1-18, (ii) a CDR2 having an amino acid sequence according to any one of SEQ ID NOs: 19-36, and (iii) a CDR3 having an amino acid sequence according to any one of SEQ ID NOs: 37-54; b) a light chain variable region (VL) comprising (i) a CDR1 having an amino acid sequence according to any one of SEQ ID NOs: 55-72, (ii) a CDR2 having an amino acid sequence according to any one of SEQ ID NOs: 73-90, and (iii) a CDR3 having an amino acid sequence according to any one of SEQ ID NOs: 91-108; and c) a Fc domain comprising amino acid modifications M252Y, S254T, and T256E (YTE) and/or M428L and N434S (LS).

Further described herein, in certain embodiments, are methods of treating an inflammatory bowel disease in a patient in need thereof, the method comprising subcutaneously or intravenously administering to the patient an effective amount of an TL1A binding protein comprising: a) a heavy chain variable region (VH) comprising (i) a CDR1 having an amino acid sequence according to any one of SEQ ID NOs: 1-18, (ii) a CDR2 having an amino acid sequence according to any one of SEQ ID NOs: 19-36, and (iii) a CDR3 having an amino acid sequence according to any one of SEQ ID NOs: 37-54; b) a light chain variable region (VL) comprising (i) a CDR1 having an amino acid sequence according to any one of SEQ ID NOs: 55-72, (ii) a CDR2 having an amino acid sequence according to any one of SEQ ID NOs: 73-90, and (iii) a CDR3 having an amino acid sequence according to any one of SEQ ID NOs: 91-108; and c) a modified Fc that extends half-life of the TL1A binding protein as compared to a TL1A binding protein that does not comprise the modified Fc.

Further described herein, in certain embodiments, are methods of treating an inflammatory bowel disease in a patient in need thereof, the method comprising subcutaneously or intravenously administering to the patient an effective amount of an TL1A binding protein, wherein the TL1A binding protein specifically binds to an epitope of TL1A and comprises a Fc domain comprising amino acid modifications M252Y, S254T, and T256E (YTE) and/or M428L and N434S (LS).

In some embodiments, the inflammatory bowel disease is Crohn's disease or ulcerative colitis. In some embodiments, the inflammatory bowel disease is ulcerative colitis.

In some embodiments, the TL1A binding protein is administered at a dose of about 75 mg to about 150 mg. In some embodiments, the TL1A binding protein is administered at a dose of about 250 mg to about 750 mg. In some embodiments, the TL1A binding protein is administered at a dose of about 300 mg to about 700 mg. In some embodiments, the TL1A binding protein is administered at a dose of about 300 mg to about 600 mg. In some embodiments, the TL1A binding protein is administered at a dose of about 300 mg to about 500 mg. In some embodiments, the TL1A binding protein is administered at a dose of about 300 mg to about 400 mg. In some embodiments, the TL1A binding protein is administered at a dose of about 400 mg to about 700 mg. In some embodiments, the TL1A binding protein is administered at a dose of about 400 mg to about 600 mg. In some embodiments, the TL1A binding protein is administered at a dose of about 300 mg to about 500 mg. In some embodiments, the TL1A binding protein is administered at a dose of about 500 mg to about 700 mg. In some embodiments, the TL1A binding protein is administered at a dose of about 500 mg to about 600 mg. In some embodiments, the TL1A binding protein is administered at a dose of about 600 mg to about 700 mg. In some embodiments, the TL1A binding protein is administered at a dose of about 75 mg, about 100 mg, about 125 mg, about 150 mg, about 175 mg, about 200 mg, about 225 mg, about 250 mg, about 275 mg, about 300 mg, about 350 mg, about 400 mg, about 450 mg, about 500 mg, about 550 mg, about 600 mg, about 650 mg, or about 700 mg.

In some embodiments, administration of the TL1A binding protein is intravenous, intratumoral, intramuscular, subcutaneous, intralesional, intraintestinal, intracolonic, intrarectal, intrapouch, or intraperitoneal. In some embodiments, administration of the TL1A binding protein is through a parenteral route such as intravenous, intramuscular, subcutaneous, intraarterial, or intraperitoneal administration. In some embodiments, administration of the TL1A binding protein is intravenous or subcutaneous. In some embodiments, administration of the TL1A binding protein is intravenous. In some embodiments, administration of the TL1A binding protein is subcutaneous.

Administration of the TL1A binding protein can occur at various intervals. In some embodiments, the TL1A binding protein is administered to the patient at least once at an interval of more than 8 weeks. In some embodiments, the interval is about 12 to about 26 weeks. In some embodiments, the TL1A binding protein, the interval is about 12 to about 22 weeks. In some embodiments, the interval is about 12 to about 18 weeks. In some embodiments, the interval is about 12 to about 14 weeks. In some embodiments, the interval is about 16 to about 26 weeks. In some embodiments, the interval is about 16 to about 22 weeks. In some embodiments, the interval is about 16 to about 18 weeks. In some embodiments, the interval is about 20 to about 26 weeks. In some embodiments, the interval is about 20 to about 22 weeks. In some embodiments, the interval is about 12 weeks. In some embodiments, the interval is about 16 weeks. In some embodiments, the interval is about 26 weeks.

### Pharmaceutical Compositions

The present disclosure also features pharmaceutical compositions that contain a therapeutically effective amount of the TL1A binding proteins described herein. The composition can be formulated for use in a variety of drug delivery systems. One or more physiologically acceptable excipients or carriers can also be included in the composition for proper formulation. Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

In some embodiments, a pharmaceutical composition may contain formulation materials for modifying, maintaining, or preserving, for example, the pH, osmolarity, viscosity, clarity, color, isotonicity, odor, sterility, stability, rate of dissolution or release, adsorption, or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants (see, Remington's Pharmaceutical Sciences, 18th ed. (Mack Publishing Company, 1990)).

In some embodiments, a pharmaceutical composition is citrate-free.

In some embodiments, a pharmaceutical composition may contain nanoparticles, e.g., polymeric nanoparticles, liposomes, or micelles.

In some embodiments, a pharmaceutical composition may contain a sustained- or controlled-delivery formulation. Techniques for formulating sustained- or controlled-delivery means, such as liposome carriers, bio-erodible microparticles or porous beads and depot injections, are also known to those skilled in the art. Sustained-release preparations may include, e.g., porous polymeric microparticles or semipermeable polymer matrices in the form of shaped articles, e.g., films, or microcapsules. Sustained release matrices may include polyesters, hydrogels, polylactides, copolymers of L-glutamic acid and gamma ethyl-L-glutamate, poly (2-hydroxyethyl-methacrylate), ethylene vinyl acetate, or poly-D(−)-3-hydroxybutyric acid. Sustained release compositions may also include liposomes that can be prepared by any of several methods known in the art.

Pharmaceutical compositions containing an TL1A binding protein disclosed herein can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, intrathecal and rectal administration. In some embodiments, the TL1A binding protein disclosed herein is administered intravenously or subcutaneously. In some embodiments, the TL1A binding protein disclosed herein is administered intravenously. In some embodiments, the TL1A binding protein disclosed herein is administered subcutaneously.

Useful formulations can be prepared by methods known in the pharmaceutical art. For example, see Remington's Pharmaceutical Sciences, 18th ed. (Mack Publishing Company, 1990). Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose. In some embodiments, the formulation for parenteral administration is citrate-free.

For intravenous or subcutaneous administration, suitable carriers include physiological saline, bacteriostatic water, Cremophor ELTM (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

An intravenous or subcutaneous drug delivery formulation may be contained in a syringe, pen, or bag. In some embodiments, the bag is connected to a channel comprising a tube and/or a needle. In some embodiments, the formulation is a lyophilized formulation or a liquid formulation.

These compositions may be sterilized by conventional sterilization techniques, or may be sterile filtered. The resulting aqueous solutions may be packaged for use as-is, or lyophilized, the lyophilized preparation being combined with a sterile aqueous carrier prior to administration.

A polyol, which acts as a tonicifier and may stabilize the TL1A binding protein, may also be included in the formulation. The polyol is added to the formulation in an amount which may vary with respect to the desired isotonicity of the formulation. In some embodiments, the aqueous formulation is isotonic. The amount of polyol added may also be altered with respect to the molecular weight of the polyol. For example, a lower amount of a monosaccharide (e.g., mannitol) is added, compared to a disaccharide (such as trehalose). In some embodiments, the polyol which is used in the formulation as a tonicity agent is mannitol.

A detergent or surfactant may also be added to the formulation. Exemplary detergents include nonionic detergents such as polysorbates (e.g., polysorbates 20, 80 etc.) or poloxamers (e.g., poloxamer 188). The amount of detergent added is such that it reduces aggregation of the formulated antibody and/or minimizes the formation of particulates in the formulation and/or reduces adsorption. In some embodiments, the formulation may include a surfactant which is a polysorbate. In some embodiments, the formulation may contain the detergent polysorbate 80 or Tween 80. Tween 80 is a term used to describe polyoxyethylene (20) sorbitan monooleate (see Fiedler, Lexikon der Hifsstoffe, Editio Cantor Verlag Aulendorf, 4th edi., 1996).

In embodiments, the protein product of the present disclosure is formulated as a liquid formulation. In some embodiments, the liquid formulation is prepared in combination with a sugar at stabilizing levels. In some embodiments, the liquid formulation is prepared in an aqueous carrier. In some embodiments, a stabilizer is added in an amount no greater than that which may result in a viscosity undesirable or unsuitable for intravenous administration. In some embodiments, the sugar is disaccharides, e.g., sucrose. In some embodiments, the liquid formulation may also include one or more of a buffering agent, a surfactant, and a preservative.

In some embodiments, the pH of the liquid formulation is set by addition of a pharmaceutically acceptable acid and/or base. In some embodiments, the pharmaceutically acceptable acid is hydrochloric acid. In some embodiments, the base is sodium hydroxide.

The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer's solution, or dextrose solution.

A preservative may be optionally added to the formulations herein to reduce bacterial action. The addition of a preservative may, for example, facilitate the production of a multi-use (multiple-dose) formulation.

The TL1A binding protein may be lyophilized to produce a lyophilized formulation including the proteins and a lyoprotectant. The lyoprotectant may be sugar, e.g., disaccharides. In some embodiments, the lyoprotectant is sucrose or maltose. The lyophilized formulation may also include one or more of a buffering agent, a surfactant, a bulking agent, and/or a preservative.

The amount of sucrose or maltose useful for stabilization of the lyophilized drug product may be in a weight ratio of at least 1:2 protein to sucrose or maltose. In some embodiments, the protein to sucrose or maltose weight ratio is of from 1:2 to 1:5. In some embodiments, the pH of the formulation, prior to lyophilization, is set by addition of a pharmaceutically acceptable acid and/or base. In some embodiments, the pharmaceutically acceptable acid is hydrochloric acid. In some embodiments, the pharmaceutically acceptable base is sodium hydroxide.

In some embodiments, the TL1A binding protein is administered at a dose of about 75 mg to about 150 mg. In some embodiments, the TL1A binding protein is administered at a dose of about 250 mg to about 750 mg. In some embodiments, the TL1A binding protein is administered at a dose of about 300 mg to about 700 mg. In some embodiments, the TL1A binding protein is administered at a dose of about 300 mg to about 600 mg. In some embodiments, the TL1A binding protein is administered at a dose of about 300 mg to about 500 mg. In some embodiments, the TL1A binding protein is administered at a dose of about 300 mg to about 400 mg. In some embodiments, the TL1A binding protein is administered at a dose of about 400 mg to about 700 mg. In some embodiments, the TL1A binding protein is administered at a dose of about 400 mg to about 600 mg. In some embodiments, the TL1A binding protein is administered at a dose of about 300 mg to about 500 mg. In some embodiments, the TL1A binding protein is administered at a dose of about 500 mg to about 700 mg. In some embodiments, the TL1A binding protein is administered at a dose of about 500 mg to about 600 mg. In some embodiments, the TL1A binding protein is administered at a dose of about 600 mg to about 700 mg. In some embodiments, the TL1A binding protein is administered at a dose of about 75 mg, about 100 mg, about 125 mg, about 150 mg, about 175 mg, about 200 mg, about 225 mg, about 250 mg, about 275 mg, about 300 mg, about 350 mg, about 400 mg, about 450 mg, about 500 mg, about 550 mg, about 600 mg, about 650 mg, or about 700 mg. In some embodiments, the TL1A binding protein is administered at a dose of about 300 mg. Actual dosage levels of the active ingredients in the pharmaceutical compositions of this disclosure may be about 250 mg, 350 mg, 400 mg, 450 mg, 500 mg, 550 mg, 600 mg, 650 mg, or 700 mg so as to obtain an amount of the active ingredient which is effective to achieve the desired therapeutic response for a particular patient, composition, and mode of administration, without being toxic to the patient.

The specific dose can be a uniform dose for each patient of about 250 mg, 300 mg, 350 mg, 400 mg, 450 mg, or 500 mg of protein. Alternatively, a patient's dose can be tailored to the approximate body weight or surface area of the patient. Other factors in determining the appropriate dosage can include the disease or condition to be treated or prevented, the severity of the disease, the route of administration, and the age, sex, and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual patient's dosage can be adjusted as the progress of the disease is monitored. Blood levels of the targetable construct or complex in a patient can be measured to see if the dosage needs to be adjusted to reach or maintain an effective concentration. Pharmacogenomics may be used to determine which targetable constructs and/or complexes, and dosages thereof, are most likely to be effective for a given individual (Schmitz et al., Clinica Chimica Acta 308: 43-53, 2001; Steimer et al., Clinica Chimica Acta 308: 33-41, 2001).

### Methods of Preparation

The TL1A binding proteins described above can be made using recombinant DNA technology well known to a skilled person in the art. For example, one or more isolated polynucleotides encoding the TL1A binding protein can be ligated to other appropriate nucleotide sequences, including, for example, constant region coding sequences, and expression control sequences, to produce conventional gene expression constructs (i.e., expression vectors) encoding the desired TL1A binding proteins. Production of defined gene constructs is within routine skill in the art.

Nucleic acids encoding desired TL1A binding proteins can be incorporated (ligated) into expression vectors, which can be introduced into host cells through conventional transfection or transformation techniques. Exemplary host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells that do not otherwise produce IgG protein. Transformed host cells can be grown under conditions that permit the host cells to express the genes that encode TL1A binding proteins.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. The expressed protein may be secreted. The expressed protein may accumulate in refractile or inclusion bodies, which can be harvested after disruption of the cells by French press or sonication. The refractile bodies then are solubilized, and the protein may be refolded and/or cleaved by methods known in the art.

If the engineered gene is to be expressed in eukaryotic host cells, e.g., CHO cells, it is first inserted into an expression vector containing a suitable eukaryotic promoter, a secretion signal, a poly A sequence, and a stop codon. Optionally, the vector or gene construct may contain enhancers and introns. In embodiments involving fusion proteins comprising an TL1A binding protein or portion thereof, the expression vector optionally contains sequences encoding all or part of a constant region, enabling an entire, or a part of, a heavy or light chain to be expressed. The gene construct can be introduced into eukaryotic host cells using conventional techniques.

In some embodiments, in order to express an TL1A binding protein, an N-terminal signal sequence is included in the protein construct. Exemplary N-terminal signal sequences include signal sequences from interleukin-2, CD-5, IgG kappa light chain, trypsinogen, serum albumin, and prolactin.

After transfection, single clones can be isolated for cell bank generation using methods known in the art, such as limited dilution, ELISA, FACS, microscopy, or Clonepix. Clones can be cultured under conditions suitable for bio-reactor scale-up and maintained expression of the TL1A binding proteins.

The TL1A binding proteins can be isolated and purified using methods known in the art including centrifugation, depth filtration, cell lysis, homogenization, freeze-thawing, affinity purification, gel filtration, ion exchange chromatography, hydrophobic interaction exchange chromatography, and mixed-mode chromatography.

### INCORPORATION BY REFERENCE

All publications and patents cited throughout the text of this specification (including all patents, patent applications, scientific publications, manufacturer's specifications, instructions, etc.), whether supra or infra, are hereby incorporated by reference in their entirety for all purposes. To the extent the material incorporated by reference contradicts or is inconsistent with this specification, the specification will supersede any such material.

### EQUIVALENTS

The disclosure may be embodied in other specific forms without departing from the spirit or essential characteristics thereof. The foregoing embodiments are therefore to be considered in all respects illustrative rather than limiting the disclosure described herein. Scope of the disclosure is thus indicated by the appended claims rather than by the foregoing description, and all changes that come within the meaning and range of equivalency of the claims are intended to be embraced therein.

1. A TL1A binding protein comprising:
a) a heavy chain variable region (VH) comprising (i) a CDR1 having an amino acid sequence according to any one of SEQ ID NOs: 1-18, (ii) a CDR2 having an amino acid sequence according to any one of SEQ ID NOs: 19-36, and (iii) a CDR3 having an amino acid sequence according to any one of SEQ ID NOs: 37-54;
b) a light chain variable region (VL) comprising (i) a CDR1 having an amino acid sequence according to any one of SEQ ID NOs: 55-72, (ii) a CDR2 having an amino acid sequence according to any one of SEQ ID NOs: 73-90, and (iii) a CDR3 having an amino acid sequence according to any one of SEQ ID NOs: 91-108; and
c) a modified Fc that extends half-life of the TL1A binding protein as compared to a TL1A binding protein that does not comprise the modified Fc.

2. The TL1A binding protein of claim 1, wherein the VH comprises (i) a CDR1 having an amino acid sequence according to SEQ ID NO: 1, (ii) a CDR2 having an amino acid sequence according to SEQ ID NO: 19, and (iii) a CDR3 having an amino acid sequence according to SEQ ID NO: 37; and the VL comprises (i) a CDR1 having an amino acid sequence according to SEQ ID NO: 55, (ii) a CDR2 having an amino acid sequence according to SEQ ID NO: 73, and (iii) a CDR3 having an amino acid sequence according to SEQ ID NO: 91, or
wherein the VH comprises (i) a CDR1 having an amino acid sequence according to SEQ ID NO: 2, (ii) a CDR2 having an amino acid sequence according to SEQ ID NO: 20, and (iii) a CDR3 having an amino acid sequence according to SEQ ID NO: 38; and the VL comprises (i) a CDR1 having an amino acid sequence according to SEQ ID NO: 56, (ii) a CDR2 having an amino acid sequence according to SEQ ID NO: 74, and (iii) a CDR3 having an amino acid sequence according to SEQ ID NO: 92, or
wherein the VH comprises (i) a CDR1 having an amino acid sequence according to SEQ ID NO: 3, (ii) a CDR2 having an amino acid sequence according to SEQ ID NO: 21, and (iii) a CDR3 having an amino acid sequence according to SEQ ID NO: 39; and the VL comprises (i) a CDR1 having an amino acid sequence according to SEQ ID NO: 57, (ii) a CDR2 having an amino acid sequence according to SEQ ID NO: 75, and (iii) a CDR3 having an amino acid sequence according to SEQ ID NO: 93, or
wherein the VH comprises (i) a CDR1 having an amino acid sequence according to SEQ ID NO: 4, (ii) a CDR2 having an amino acid sequence according to SEQ ID NO: 22, and (iii) a CDR3 having an amino acid sequence according to SEQ ID NO: 40; and the VL comprises (i) a CDR1 having an amino acid sequence according to SEQ ID NO: 58, (ii) a CDR2 having an amino acid sequence according to SEQ ID NO: 76, and (iii) a CDR3 having an amino acid sequence according to SEQ ID NO: 94, or
wherein the VH comprises (i) a CDR1 having an amino acid sequence according to SEQ ID NO: 5, (ii) a CDR2 having an amino acid sequence according to SEQ ID NO: 23, and (iii) a CDR3 having an amino acid sequence according to SEQ ID NO: 41; and the VL comprises (i) a CDR1 having an amino acid sequence according to SEQ ID NO: 59, (ii) a CDR2 having an amino acid sequence according to SEQ ID NO: 77, and (iii) a CDR3 having an amino acid sequence according to SEQ ID NO: 95, or
wherein the VH comprises (i) a CDR1 having an amino acid sequence according to SEQ ID NO: 6, (ii) a CDR2 having an amino acid sequence according to SEQ ID NO: 24, and (iii) a CDR3 having an amino acid sequence according to SEQ ID NO: 42; and the VL comprises (i) a CDR1 having an amino acid sequence according to SEQ ID NO: 60, (ii) a CDR2 having an amino acid sequence according to SEQ ID NO: 78, and (iii) a CDR3 having an amino acid sequence according to SEQ ID NO: 96, or
wherein the VH comprises (i) a CDR1 having an amino acid sequence according to SEQ ID NO: 7, (ii) a CDR2 having an amino acid sequence according to SEQ ID NO: 25, and (iii) a CDR3 having an amino acid sequence according to SEQ ID NO: 43; and the VL comprises (i) a CDR1 having an amino acid sequence according to SEQ ID NO: 61, (ii) a CDR2 having an amino acid sequence according to SEQ ID NO: 79, and (iii) a CDR3 having an amino acid sequence according to SEQ ID NO: 97, or
wherein the VH comprises (i) a CDR1 having an amino acid sequence according to SEQ ID NO: 8, (ii) a CDR2 having an amino acid sequence according to SEQ ID NO: 26, and (iii) a CDR3 having an amino acid sequence according to SEQ ID NO: 44; and the VL comprises (i) a CDR1 having an amino acid sequence according to SEQ ID NO: 62, (ii) a CDR2 having an amino acid sequence according to SEQ ID NO: 80, and (iii) a CDR3 having an amino acid sequence according to SEQ ID NO: 98, or
wherein the VH comprises (i) a CDR1 having an amino acid sequence according to SEQ ID NO: 9, (ii) a CDR2 having an amino acid sequence according to SEQ ID NO: 27, and (iii) a CDR3 having an amino acid sequence according to SEQ ID NO: 45; and the VL comprises (i) a CDR1 having an amino acid sequence according to SEQ ID NO: 63, (ii) a CDR2 having an amino acid sequence according to SEQ ID NO: 81, and (iii) a CDR3 having an amino acid sequence according to SEQ ID NO: 99, or
wherein the VH comprises (i) a CDR1 having an amino acid sequence according to SEQ ID NO: 10, (ii) a CDR2 having an amino acid sequence according to SEQ ID NO: 28, and (iii) a CDR3 having an amino acid sequence according to SEQ ID NO: 46; and the VL comprises (i) a CDR1 having an amino acid sequence according to SEQ ID NO: 64, (ii) a CDR2 having an amino acid sequence according to SEQ ID NO: 82, and (iii) a CDR3 having an amino acid sequence according to SEQ ID NO: 100, or
wherein the VH comprises (i) a CDR1 having an amino acid sequence according to SEQ ID NO: 11, (ii) a CDR2 having an amino acid sequence according to SEQ ID NO: 29, and (iii) a CDR3 having an amino acid sequence according to SEQ ID NO: 47; and the VL comprises (i) a CDR1 having an amino acid sequence according to SEQ ID NO: 65, (ii) a CDR2 having an amino acid sequence according to SEQ ID NO: 83, and (iii) a CDR3 having an amino acid sequence according to SEQ ID NO: 101, or
wherein the VH comprises (i) a CDR1 having an amino acid sequence according to SEQ ID NO: 12, (ii) a CDR2 having an amino acid sequence according to SEQ ID NO: 30, and (iii) a CDR3 having an amino acid sequence according to SEQ ID NO: 48; and the VL comprises (i) a CDR1 having an amino acid sequence according to SEQ ID NO: 66, (ii) a CDR2 having an amino acid sequence according to SEQ ID NO: 84, and (iii) a CDR3 having an amino acid sequence according to SEQ ID NO: 102, or
wherein the VH comprises (i) a CDR1 having an amino acid sequence according to SEQ ID NO: 13, (ii) a CDR2 having an amino acid sequence according to SEQ ID NO: 31, and (iii) a CDR3 having an amino acid sequence according to SEQ ID NO: 49; and the VL comprises (i) a CDR1 having an amino acid sequence according to SEQ ID NO: 67, (ii) a CDR2 having an amino acid sequence according to SEQ ID NO: 85, and (iii) a CDR3 having an amino acid sequence according to SEQ ID NO: 103, or
wherein the VH comprises (i) a CDR1 having an amino acid sequence according to SEQ ID NO: 14, (ii) a CDR2 having an amino acid sequence according to SEQ ID NO: 32, and (iii) a CDR3 having an amino acid sequence according to SEQ ID NO: 50; and the VL comprises (i) a CDR1 having an amino acid sequence according to SEQ ID NO: 68, (ii) a CDR2 having an amino acid sequence according to SEQ ID NO: 86, and (iii) a CDR3 having an amino acid sequence according to SEQ ID NO: 104, or
wherein the VH comprises (i) a CDR1 having an amino acid sequence according to SEQ ID NO: 15, (ii) a CDR2 having an amino acid sequence according to SEQ ID NO: 33, and (iii) a CDR3 having an amino acid sequence according to SEQ ID NO: 51; and the VL comprises (i) a CDR1 having an amino acid sequence according to SEQ ID NO: 69, (ii) a CDR2 having an amino acid sequence according to SEQ ID NO: 87, and (iii) a CDR3 having an amino acid sequence according to SEQ ID NO: 105, or
wherein the VH comprises (i) a CDR1 having an amino acid sequence according to SEQ ID NO: 16, (ii) a CDR2 having an amino acid sequence according to SEQ ID NO: 34, and (iii) a CDR3 having an amino acid sequence according to SEQ ID NO: 52; and the VL comprises (i) a CDR1 having an amino acid sequence according to SEQ ID NO: 70, (ii) a CDR2 having an amino acid sequence according to SEQ ID NO: 88, and (iii) a CDR3 having an amino acid sequence according to SEQ ID NO: 106, or
wherein the VH comprises (i) a CDR1 having an amino acid sequence according to SEQ ID NO: 17, (ii) a CDR2 having an amino acid sequence according to SEQ ID NO: 35, and (iii) a CDR3 having an amino acid sequence according to SEQ ID NO: 53; and the VL comprises (i) a CDR1 having an amino acid sequence according to SEQ ID NO: 71, (ii) a CDR2 having an amino acid sequence according to SEQ ID NO: 89, and (iii) a CDR3 having an amino acid sequence according to SEQ ID NO: 107, or
wherein the VH comprises (i) a CDR1 having an amino acid sequence according to SEQ ID NO: 18, (ii) a CDR2 having an amino acid sequence according to SEQ ID NO: 36, and (iii) a CDR3 having an amino acid sequence according to SEQ ID NO: 54; and the VL comprises (i) a CDR1 having an amino acid sequence according to SEQ ID NO: 72, (ii) a CDR2 having an amino acid sequence according to SEQ ID NO: 90, and (iii) a CDR3 having an amino acid sequence according to SEQ ID NO: 108.

3. The TL1A binding protein of claim 2, wherein the VH comprises a sequence having at least 80% sequence identity to the amino acid sequence of any one of SEQ ID NOs: 325-342 and the VL comprises a sequence having at least 80% sequence identity to the amino acid sequence of any one of SEQ ID NOs: 343-360, or
wherein the VH comprises a sequence having at least 80% sequence identity to the amino acid sequence of SEQ ID NO: 325 and the VL comprises a sequence having at least 80% sequence identity to the amino acid sequence of SEQ ID NO: 343, or
wherein the VH comprises a sequence having at least 80% sequence identity to the amino acid sequence of SEQ ID NO: 326 and the VL comprises a sequence having at least 80% sequence identity to the amino acid sequence of SEQ ID NO: 344, or
wherein the VH comprises a sequence having at least 80% sequence identity to the amino acid sequence of SEQ ID NO: 327 and the VL comprises a sequence having at least 80% sequence identity to the amino acid sequence of SEQ ID NO: 345, or
wherein the VH comprises a sequence having at least 80% sequence identity to the amino acid sequence of SEQ ID NO: 328 and the VL comprises a sequence having at least 80% sequence identity to the amino acid sequence of SEQ ID NO: 346, or
wherein the VH comprises a sequence having at least 80% sequence identity to the amino acid sequence of SEQ ID NO: 329 and the VL comprises a sequence having at least 80% sequence identity to the amino acid sequence of SEQ ID NO: 347, or
wherein the VH comprises a sequence having at least 80% sequence identity to the amino acid sequence of SEQ ID NO: 330 and the VL comprises a sequence having at least 80% sequence identity to the amino acid sequence of SEQ ID NO: 348, or
wherein the VH comprises a sequence having at least 80% sequence identity to the amino acid sequence of SEQ ID NO: 331 and the VL comprises a sequence having at least 80% sequence identity to the amino acid sequence of SEQ ID NO: 349, or
wherein the VH comprises a sequence having at least 80% sequence identity to the amino acid sequence of SEQ ID NO: 332 and the VL comprises a sequence having at least 80% sequence identity to the amino acid sequence of SEQ ID NO: 350, or
wherein the VH comprises a sequence having at least 80% sequence identity to the amino acid sequence of SEQ ID NO: 333 and the VL comprises a sequence having at least 80% sequence identity to the amino acid sequence of SEQ ID NO: 351, or
wherein the VH comprises a sequence having at least 80% sequence identity to the amino acid sequence of SEQ ID NO: 334 and the VL comprises a sequence having at least 80% sequence identity to the amino acid sequence of SEQ ID NO: 352, or
wherein the VH comprises a sequence having at least 80% sequence identity to the amino acid sequence of SEQ ID NO: 335 and the VL comprises a sequence having at least 80% sequence identity to the amino acid sequence of SEQ ID NO: 353, or
wherein the VH comprises a sequence having at least 80% sequence identity to the amino acid sequence of SEQ ID NO: 336 and the VL comprises a sequence having at least 80% sequence identity to the amino acid sequence of SEQ ID NO: 354, or
wherein the VH comprises a sequence having at least 80% sequence identity to the amino acid sequence of SEQ ID NO: 337 and the VL comprises a sequence having at least 80% sequence identity to the amino acid sequence of SEQ ID NO: 355, or
wherein the VH comprises a sequence having at least 80% sequence identity to the amino acid sequence of SEQ ID NO: 338 and the VL comprises a sequence having at least 80% sequence identity to the amino acid sequence of SEQ ID NO: 356, or
wherein the VH comprises a sequence having at least 80% sequence identity to the amino acid sequence of SEQ ID NO: 339 and the VL comprises a sequence having at least 80% sequence identity to the amino acid sequence of SEQ ID NO: 357, or
wherein the VH comprises a sequence having at least 80% sequence identity to the amino acid sequence of SEQ ID NO: 340 and the VL comprises a sequence having at least 80% sequence identity to the amino acid sequence of SEQ ID NO: 358, or
wherein the VH comprises a sequence having at least 80% sequence identity to the amino acid sequence of SEQ ID NO: 341 and the VL comprises a sequence having at least 80% sequence identity to the amino acid sequence of SEQ ID NO: 359, or
wherein the VH comprises a sequence having at least 80% sequence identity to the amino acid sequence of SEQ ID NO: 342 and the VL comprises a sequence having at least 80% sequence identity to the amino acid sequence of SEQ ID NO: 360.

4. The TL1A binding protein of claim 1, wherein the modified Fc domain is an Fc domain comprising amino acid modifications M252Y, S254T, and T256E (YTE) according to EU numbering system.

5. The TL1A binding protein of claim 1, wherein the modified Fc domain is an Fc domain comprising amino acid modifications M428L and N434S (LS) according to EU numbering system.

6. The TL1A binding protein of claim 1, wherein the modified Fc domain is an Fc domain comprising amino acid modifications M252Y, S254T, and T256E (YTE) and M428L and N434S (LS) according to EU numbering system.

7. The TL1A binding protein of claim 1, wherein the Fc is an IgG1 immunoglobulin domain.

8. The TL1A binding protein of claim 1, wherein the Fc is an IgG2 immunoglobulin domain.

9. The TL1A binding protein of claim 1, wherein the Fc is an IgG4 immunoglobulin domain.

10. A TL1A binding protein comprising:
a) a heavy chain variable region (VH) comprising (i) a CDR1 having an amino acid sequence according to any one of SEQ ID NOs: 1-18, (ii) a CDR2 having an amino acid sequence according to any one of SEQ ID NOs: 19-36, and (iii) a CDR3 having an amino acid sequence according to any one of SEQ ID NOs: 37-54;
b) a light chain variable region (VL) comprising (i) a CDR1 having an amino acid sequence according to any one of SEQ ID NOs: 55-72, (ii) a CDR2 having an amino acid sequence according to any one of SEQ ID NOs: 73-90, and (iii) a CDR3 having an amino acid sequence according to any one of SEQ ID NOs: 91-108; and
c) an Fc domain comprising amino acid modifications M252Y, S254T, and T256E (YTE) and/or M428L and N434S (LS) according to EU numbering system.

11. The TL1A binding protein of claim 10, wherein the VH comprises (i) a CDR1 having an amino acid sequence according to SEQ ID NO: 1, (ii) a CDR2 having an amino acid sequence according to SEQ ID NO: 19, and (iii) a CDR3 having an amino acid sequence according to SEQ ID NO: 37; and the VL comprises (i) a CDR1 having an amino acid sequence according to SEQ ID NO: 55, (ii) a CDR2 having an amino acid sequence according to SEQ ID NO: 73, and (iii) a CDR3 having an amino acid sequence according to SEQ ID NO: 91, or
wherein the VH comprises (i) a CDR1 having an amino acid sequence according to SEQ ID NO: 2, (ii) a CDR2 having an amino acid sequence according to SEQ ID NO: 20, and (iii) a CDR3 having an amino acid sequence according to SEQ ID NO: 38; and the VL comprises (i) a CDR1 having an amino acid sequence according to SEQ ID NO: 56, (ii) a CDR2 having an amino acid sequence according to SEQ ID NO: 74, and (iii) a CDR3 having an amino acid sequence according to SEQ ID NO: 92, or
wherein the VH comprises (i) a CDR1 having an amino acid sequence according to SEQ ID NO: 3, (ii) a CDR2 having an amino acid sequence according to SEQ ID NO: 21, and (iii) a CDR3 having an amino acid sequence according to SEQ ID NO: 39; and the VL comprises (i) a CDR1 having an amino acid sequence according to SEQ ID NO: 57, (ii) a CDR2 having an amino acid sequence according to SEQ ID NO: 75, and (iii) a CDR3 having an amino acid sequence according to SEQ ID NO: 93, or
wherein the VH comprises (i) a CDR1 having an amino acid sequence according to SEQ ID NO: 4, (ii) a CDR2 having an amino acid sequence according to SEQ ID NO: 22, and (iii) a CDR3 having an amino acid sequence according to SEQ ID NO: 40; and the VL comprises (i) a CDR1 having an amino acid sequence according to SEQ ID NO: 58, (ii) a CDR2 having an amino acid sequence according to SEQ ID NO: 76, and (iii) a CDR3 having an amino acid sequence according to SEQ ID NO: 94, or
wherein the VH comprises (i) a CDR1 having an amino acid sequence according to SEQ ID NO: 5, (ii) a CDR2 having an amino acid sequence according to SEQ ID NO: 23, and (iii) a CDR3 having an amino acid sequence according to SEQ ID NO: 41; and the VL comprises (i) a CDR1 having an amino acid sequence according to SEQ ID NO: 59, (ii) a CDR2 having an amino acid sequence according to SEQ ID NO: 77, and (iii) a CDR3 having an amino acid sequence according to SEQ ID NO: 95, or
wherein the VH comprises (i) a CDR1 having an amino acid sequence according to SEQ ID NO: 6, (ii) a CDR2 having an amino acid sequence according to SEQ ID NO: 24, and (iii) a CDR3 having an amino acid sequence according to SEQ ID NO: 42; and the VL comprises (i) a CDR1 having an amino acid sequence according to SEQ ID NO: 60, (ii) a CDR2 having an amino acid sequence according to SEQ ID NO: 78, and (iii) a CDR3 having an amino acid sequence according to SEQ ID NO: 96, or
wherein the VH comprises (i) a CDR1 having an amino acid sequence according to SEQ ID NO: 7, (ii) a CDR2 having an amino acid sequence according to SEQ ID NO: 25, and (iii) a CDR3 having an amino acid sequence according to SEQ ID NO: 43; and the VL comprises (i) a CDR1 having an amino acid sequence according to SEQ ID NO: 61, (ii) a CDR2 having an amino acid sequence according to SEQ ID NO: 79, and (iii) a CDR3 having an amino acid sequence according to SEQ ID NO: 97, or
wherein the VH comprises (i) a CDR1 having an amino acid sequence according to SEQ ID NO: 8, (ii) a CDR2 having an amino acid sequence according to SEQ ID NO: 26, and (iii) a CDR3 having an amino acid sequence according to SEQ ID NO: 44; and the VL comprises (i) a CDR1 having an amino acid sequence according to SEQ ID NO: 62, (ii) a CDR2 having an amino acid sequence according to SEQ ID NO: 80, and (iii) a CDR3 having an amino acid sequence according to SEQ ID NO: 98, or
wherein the VH comprises (i) a CDR1 having an amino acid sequence according to SEQ ID NO: 9, (ii) a CDR2 having an amino acid sequence according to SEQ ID NO: 27, and (iii) a CDR3 having an amino acid sequence according to SEQ ID NO: 45; and the VL comprises (i) a CDR1 having an amino acid sequence according to SEQ ID NO: 63, (ii) a CDR2 having an amino acid sequence according to SEQ ID NO: 81, and (iii) a CDR3 having an amino acid sequence according to SEQ ID NO: 99, or
wherein the VH comprises (i) a CDR1 having an amino acid sequence according to SEQ ID NO: 10, (ii) a CDR2 having an amino acid sequence according to SEQ ID NO: 28, and (iii) a CDR3 having an amino acid sequence according to SEQ ID NO: 46; and the VL comprises (i) a CDR1 having an amino acid sequence according to SEQ ID NO: 64, (ii) a CDR2 having an amino acid sequence according to SEQ ID NO: 82, and (iii) a CDR3 having an amino acid sequence according to SEQ ID NO: 100, or
wherein the VH comprises (i) a CDR1 having an amino acid sequence according to SEQ ID NO: 11, (ii) a CDR2 having an amino acid sequence according to SEQ ID NO: 29, and (iii) a CDR3 having an amino acid sequence according to SEQ ID NO: 47; and the VL comprises (i) a CDR1 having an amino acid sequence according to SEQ ID NO: 65, (ii) a CDR2 having an amino acid sequence according to SEQ ID NO: 83, and (iii) a CDR3 having an amino acid sequence according to SEQ ID NO: 101, or
wherein the VH comprises (i) a CDR1 having an amino acid sequence according to SEQ ID NO: 12, (ii) a CDR2 having an amino acid sequence according to SEQ ID NO: 30, and (iii) a CDR3 having an amino acid sequence according to SEQ ID NO: 48; and the VL comprises (i) a CDR1 having an amino acid sequence according to SEQ ID NO: 66, (ii) a CDR2 having an amino acid sequence according to SEQ ID NO: 84, and (iii) a CDR3 having an amino acid sequence according to SEQ ID NO: 102, or
wherein the VH comprises (i) a CDR1 having an amino acid sequence according to SEQ ID NO: 13, (ii) a CDR2 having an amino acid sequence according to SEQ ID NO: 31, and (iii) a CDR3 having an amino acid sequence according to SEQ ID NO: 49; and the VL comprises (i) a CDR1 having an amino acid sequence according to SEQ ID NO: 67, (ii) a CDR2 having an amino acid sequence according to SEQ ID NO: 85, and (iii) a CDR3 having an amino acid sequence according to SEQ ID NO: 103, or
wherein the VH comprises (i) a CDR1 having an amino acid sequence according to SEQ ID NO: 14, (ii) a CDR2 having an amino acid sequence according to SEQ ID NO: 32, and (iii) a CDR3 having an amino acid sequence according to SEQ ID NO: 50; and the VL comprises (i) a CDR1 having an amino acid sequence according to SEQ ID NO: 68, (ii) a CDR2 having an amino acid sequence according to SEQ ID NO: 86, and (iii) a CDR3 having an amino acid sequence according to SEQ ID NO: 104, or
wherein the VH comprises (i) a CDR1 having an amino acid sequence according to SEQ ID NO: 15, (ii) a CDR2 having an amino acid sequence according to SEQ ID NO: 33, and (iii) a CDR3 having an amino acid sequence according to SEQ ID NO: 51; and the VL comprises (i) a CDR1 having an amino acid sequence according to SEQ ID NO: 69, (ii) a CDR2 having an amino acid sequence according to SEQ ID NO: 87, and (iii) a CDR3 having an amino acid sequence according to SEQ ID NO: 105, or
wherein the VH comprises (i) a CDR1 having an amino acid sequence according to SEQ ID NO: 16, (ii) a CDR2 having an amino acid sequence according to SEQ ID NO: 34, and (iii) a CDR3 having an amino acid sequence according to SEQ ID NO: 52; and the VL comprises (i) a CDR1 having an amino acid sequence according to SEQ ID NO: 70, (ii) a CDR2 having an amino acid sequence according to SEQ ID NO: 88, and (iii) a CDR3 having an amino acid sequence according to SEQ ID NO: 106, or
wherein the VH comprises (i) a CDR1 having an amino acid sequence according to SEQ ID NO: 17, (ii) a CDR2 having an amino acid sequence according to SEQ ID NO: 35, and (iii) a CDR3 having an amino acid sequence according to SEQ ID NO: 53; and the VL comprises (i) a CDR1 having an amino acid sequence according to SEQ ID NO: 71, (ii) a CDR2 having an amino acid sequence according to SEQ ID NO: 89, and (iii) a CDR3 having an amino acid sequence according to SEQ ID NO: 107, or
wherein the VH comprises (i) a CDR1 having an amino acid sequence according to SEQ ID NO: 18, (ii) a CDR2 having an amino acid sequence according to SEQ ID NO: 36, and (iii) a CDR3 having an amino acid sequence according to SEQ ID NO: 54; and the VL comprises (i) a CDR1 having an amino acid sequence according to SEQ ID NO: 72, (ii) a CDR2 having an amino acid sequence according to SEQ ID NO: 90, and (iii) a CDR3 having an amino acid sequence according to SEQ ID NO: 108.

12. The TL1A binding protein of claim 11, wherein the VH comprises a sequence having at least 80% sequence identity to the amino acid sequence of any one of SEQ ID NOs: 325-342 and the VL comprises a sequence having at least 80% sequence identity to the amino acid sequence of any one of SEQ ID NOs: 343-360, or
wherein the VH comprises a sequence having at least 80% sequence identity to the amino acid sequence of SEQ ID NO: 325 and the VL comprises a sequence having at least 80% sequence identity to the amino acid sequence of SEQ ID NO: 343, or
wherein the VH comprises a sequence having at least 80% sequence identity to the amino acid sequence of SEQ ID NO: 326 and the VL comprises a sequence having at least 80% sequence identity to the amino acid sequence of SEQ ID NO: 344, or
wherein the VH comprises a sequence having at least 80% sequence identity to the amino acid sequence of SEQ ID NO: 327 and the VL comprises a sequence having at least 80% sequence identity to the amino acid sequence of SEQ ID NO: 345, or
wherein the VH comprises a sequence having at least 80% sequence identity to the amino acid sequence of SEQ ID NO: 328 and the VL comprises a sequence having at least 80% sequence identity to the amino acid sequence of SEQ ID NO: 346, or
wherein the VH comprises a sequence having at least 80% sequence identity to the amino acid sequence of SEQ ID NO: 329 and the VL comprises a sequence having at least 80% sequence identity to the amino acid sequence of SEQ ID NO: 347, or
wherein the VH comprises a sequence having at least 80% sequence identity to the amino acid sequence of SEQ ID NO: 330 and the VL comprises a sequence having at least 80% sequence identity to the amino acid sequence of SEQ ID NO: 348, or
wherein the VH comprises a sequence having at least 80% sequence identity to the amino acid sequence of SEQ ID NO: 331 and the VL comprises a sequence having at least 80% sequence identity to the amino acid sequence of SEQ ID NO: 349, or
wherein the VH comprises a sequence having at least 80% sequence identity to the amino acid sequence of SEQ ID NO: 332 and the VL comprises a sequence having at least 80% sequence identity to the amino acid sequence of SEQ ID NO: 350, or
wherein the VH comprises a sequence having at least 80% sequence identity to the amino acid sequence of SEQ ID NO: 333 and the VL comprises a sequence having at least 80% sequence identity to the amino acid sequence of SEQ ID NO: 351, or
wherein the VH comprises a sequence having at least 80% sequence identity to the amino acid sequence of SEQ ID NO: 334 and the VL comprises a sequence having at least 80% sequence identity to the amino acid sequence of SEQ ID NO: 352, or
wherein the VH comprises a sequence having at least 80% sequence identity to the amino acid sequence of SEQ ID NO: 335 and the VL comprises a sequence having at least 80% sequence identity to the amino acid sequence of SEQ ID NO: 353, or
wherein the VH comprises a sequence having at least 80% sequence identity to the amino acid sequence of SEQ ID NO: 336 and the VL comprises a sequence having at least 80% sequence identity to the amino acid sequence of SEQ ID NO: 354, or
wherein the VH comprises a sequence having at least 80% sequence identity to the amino acid sequence of SEQ ID NO: 337 and the VL comprises a sequence having at least 80% sequence identity to the amino acid sequence of SEQ ID NO: 355, or
wherein the VH comprises a sequence having at least 80% sequence identity to the amino acid sequence of SEQ ID NO: 338 and the VL comprises a sequence having at least 80% sequence identity to the amino acid sequence of SEQ ID NO: 356, or
wherein the VH comprises a sequence having at least 80% sequence identity to the amino acid sequence of SEQ ID NO: 339 and the VL comprises a sequence having at least 80% sequence identity to the amino acid sequence of SEQ ID NO: 357, or
wherein the VH comprises a sequence having at least 80% sequence identity to the amino acid sequence of SEQ ID NO: 340 and the VL comprises a sequence having at least 80% sequence identity to the amino acid sequence of SEQ ID NO: 358, or
wherein the VH comprises a sequence having at least 80% sequence identity to the amino acid sequence of SEQ ID NO: 341 and the VL comprises a sequence having at least 80% sequence identity to the amino acid sequence of SEQ ID NO: 359, or
wherein the VH comprises a sequence having at least 80% sequence identity to the amino acid sequence of SEQ ID NO: 342 and the VL comprises a sequence having at least 80% sequence identity to the amino acid sequence of SEQ ID NO: 360.

13. A TL1A binding protein, wherein the TL1A binding protein specifically binds to an epitope of TL1A and comprises a Fc domain comprising amino acid modifications M252Y, S254T, and T256E (YTE) and/or M428L and N434S (LS) according to EU numbering system.

14. A method of treating an inflammatory bowel disease in a patient in need thereof, the method comprising subcutaneously or intravenously administering to the patient an effective amount of an TL1A binding protein of claim 1.

15. The method of claim 14, wherein the inflammatory bowel disease is ulcerative colitis.

16. The method of claim 14, wherein the inflammatory bowel disease is Crohn's disease.

17. The method of claim 14, wherein administration of the TL1A binding protein is subcutaneous.

18. The method of claim 14, wherein administration of the TL1A binding protein is intravenous.
